SEQUENCE LISTING (99 sequences)

SEQ ID NO: 1 - protein, 469 aa, Artificial Sequence
  Synthetic
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 2 - protein, 469 aa, Artificial Sequence
  Synthetic
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 3 - protein, 469 aa, Artificial Sequence
  Synthetic
MNPNQKIITIGVVNTTLSTIALLIGVGNLVFNTVIHEKIGDHQIVTYPIITTPAVPNCSDTIITYNNTVINNITTTIITEEERPFKSPLPLCPFRGFFPFHKDNAIRLGENKDVIVTREPYVSCDNDNCWSFALAQGALLGTKHSNGTIKDRTPYRSLIRFPIGTAPVLGNYKEICIAWSSSSCFDGKEWMHVCMTGNDNDASAQIIYGGRMTDSIKSWRKDILRTQESECQCIDGTCVVAVTDGPAANSADYRVYWIREGKIIKYENVPKTKIQHLEECSCYVDIDVYCICRDNWKGSNRPWMRINNETILETGYVCSKFHSDTPRPADPSTMSCDSPSNVNGGPGVKGFGFKAGDDVWLGRTVSTSGRSGFEIIKVTEGWINSPNHVKSITQTLVSNNDWSGYSGSFIVKAKDCFQPCFYVELIRGRPNKNDDVSWTSNSIVTFCGLDNEPGSGNWPDGSNIGFMPK

SEQ ID NO: 4 - protein, 470 aa, Artificial Sequence
  Synthetic
MNPNQKIITIGSVSIILTTVGLLLQITSLCSIWFSHYNQVAQTHEQPCSNNTTNYYNETFVNVTNVQNNYTTIAEPSAPDVVHYSSGRDLCPIRGWAPLSKDNGIRIGSRGEVFVIREPFISCSISECRTFFLTQGALLNDKHSNGTVKDRSPFRTLMSCPIGVAPSPSNSRFESVAWSATACSDGPGWLTLGITGPDSTAVAVLKYNGIITDTLKSWKGNIMRTQESECVCQDEFCYTLVTDGPSDAQAFYKILKIRKGKIVSMKDVDATGFHFEECSCYPSGTEIECVCRDNWRGSNRPWIRFNSDLDYQIGYVCSGIFGDNPRPVDGTGSCNGPVNNGKGRYGVKGFSFRYGDGVWIGRTKSLESRSGFEMVWDANGWVSTDKDSNGVQDIIDNDNWSGYSGSFSIRGETTGKNCTVPCFWVEMIRGQPKEKTIWTSGSSIAFCGVNSDTTGWSWPDGALLPFDIDK

SEQ ID NO: 5 - protein, 473 aa, Artificial Sequence
  Synthetic
MNPNQKIITIGSVSLALVVFNILLHIASIVIGIISVTKEISVSSTCNTTEVYNETVRLETITIPINNTVYIERESHQEPEFLNNTEPLCNVSGFAIVSKDNGIRIGSRGHVFVIREPFVACGPTECRTFFLTQGALLNDKHSNNTVKDRSPYRALMSVPLGSSPNAYQAKFESVAWSATACHDGKRWLAVGISGADDDAYAVIHYGGMPTDVVRSWRKQILRTQESSCVCMKGNCYWVMTDGPANSQASYKIFKSHKGMVTNEREVSFQGGHIEECSCYPNLGKVECVCRDNWNGMNRPVLTFDEDLNYEVGYLCAGIPTDTPRVQDNSFIGSCTNAVGGSGTNNYGVKGFGFRQGNSVWAGRTVSISSRSGFEILLVEDGWVKTSKNVVKKVEVLNNKNWSGYSGAFTIPITMTSKQCLVPCFWLEMIRGKPEERTSIWTSSSSTVFCGVSSEVPGWSWDDGAILPFDIDKM

SEQ ID NO: 6 - protein, 470 aa, Artificial Sequence
  Synthetic
MNPNQKITCISATGMTLSVVSMLIGIANLGLNIGLHYKVSDSTNINIPNMNETSPTTTIINNHPQNNFTNITNIIVTKTEEGHLLNLTKPLCEVNSWHILSKDNAIRIGEDAHIIVTREPYLSCDPQGCRMFALSQGTTLRGKHANGTIHDRSPFRALVSWEMGQAPSPYNTRVECIGWSSTSCHDGISRMSICISGPNNNASAVVWYGGRPVTEIPSWAGNILRTQESECVCHGGICPVVMTDGPANNRAETKIIYFKEGKIKKIEELKGDAQHIEECSCYGASEMIKCICRDNWKGANRPVITIDPEMMTHTSKYLCSKILTDTSRPNDPTNGKCEAPITGGSPDPGVKGFAFLDGENSWLGRTISKDSRSGYEMLKVPNAETDTQSGAISHQIIVNNQNWSGYSGAFIDYWANKECFNPCFYVELIRGRPKESSVLWTSNSIVALCGSKERLGSWSWHDGAEIIYFK

SEQ ID NO: 7 - protein, 471 aa, Artificial Sequence
  Synthetic
MNPNQKLFALSGVAIALSVLNLLIGISNVGLNVSLHLKEKGPKQEENLTCTTINQNNTTVVENTYVNNTTIITKGTDLKTPSYLLLNKSLCNVEGWVVIAKDNAVRFGESEQIIVTREPYVSCDPTGCKMYALHQGTTIRNKHSNGTIHDRTAFRGLISTPLGTPPTVSNSDFMCVGWSSTTCHDGIARMTICIQGNNDNATATVYYNRRLTTTIKTWARNILRTQESECVCHNGTCAVVMTDGSASSQAYTKVMYFHKGLVVKEEELRGSARHIEECSCYGHNQKVTCVCRDNWQGANRPIIEIDMSTLEHTSRYVCTGILTDTSRPGDKSSGDCSNPITGSPGVPGVKGFGFLNGDNTWLGRTISPRSRSGFEMLKIPNAGTDPNSRIAERQEIVDNNNWSGYSGSFIDYWNDNSECYNPCFYVELIRGRPEEAKYVWWASNSLIALCGSPFPVGSGSFPDGAQIQYFS

SEQ ID NO: 8 - protein, 470 aa, Artificial Sequence
  Synthetic
MNPNQKIITIGSVSLGLVILNILLHIVSITVTVLVLPGNGNNESCNETVIREYNETVRVEKVTQWHNTNVIEYIERPENDHFMNNTEALCDAKGFAPFSKDNGIRIGSRGHVFVIREPFVSCSPTECRTFFLTQGSLLNDKHSNGTVKDRSPYRTLMSVEIGQSPNVYQARFEAVAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCKDNWTGTNRPVLVISPDLSYRVGYLCAGLPSDTPRGEDSQFTGSCTSPMGNQGYGVKGFGFRQGNDVWMGRTISRTSRSGFEILKVRNGWVQNSKEQIKRQVVVDNLNWSGYSGSFTLPAELTKRNCLVPCFWVEMIRGNPEEKTIWTSSSSIVMCGVDHEIADWSWHDGAILPFDIDKM

SEQ ID NO: 9 - protein, 464 aa, Artificial Sequence
  Synthetic
MNPNQKILCTSATAIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 10 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGEVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 11 - protein, 387 aa, Artificial Sequence
  Synthetic
VKLAGNSSLCPVSGWAPLSKDNSVRIGSKGEVFVIREPFISCSPLECRTFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTTGWSWPDGAELPFTIDK

SEQ ID NO: 12 - protein, 387 aa, Artificial Sequence
  Synthetic
VKLAGNSSLCPVSGWAPYSKDNSVRIGSKGEVFVIREPFISCSPLECRTFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTTGWSWPDGAELPFTIDK

SEQ ID NO: 13 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 14 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 15 - protein, 387 aa, Artificial Sequence
  Synthetic
VKLAGNSSLCPVSGWAPLSKNNAVRIGSKGDVFVIREPFISCSPLECRTFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 16 - protein, 387 aa, Artificial Sequence
  Synthetic
VKLAGNSSLCPVSGWAPLSKDNSVRIGSKGDIFVIREPFISCSPLECRTFFLTQGALLNDKHSNGTIKDRSPYRTLMSVPIGSPPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 17 - protein, 387 aa, Artificial Sequence
  Synthetic
VKLAGNSSLCPVSGWAPLCKDNSVRIGSKGDVFVIREPFVSCSPLECRTFFLTQGALLNDKHSNGTIKDRSPYRTLMSVPICSVPSPYNARVESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSIVFCGVNSDTVGWSWPDGAELPFTIDK

SEQ ID NO: 18 - protein, 387 aa, Artificial Sequence
  Synthetic
VKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 19 - protein, 387 aa, Artificial Sequence
  Synthetic
VKLAGNSSLCPVSGWAPLSKNNAVRIGSKGDVFVIREPFISCSPLECRQFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 20 - protein, 387 aa, Artificial Sequence
  Synthetic
VKLAGNSSLCPVSGWAPLSKDNSVRIGSKGDIFVIREPFISCSPLECRQFFLTQGALLNDKHSNGTIKDRSPYRTLMSVPIGSPPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 21 - protein, 387 aa, Artificial Sequence
  Synthetic
VKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTVGWSWPDGAELPFTIDK

SEQ ID NO: 22 - protein, 387 aa, Artificial Sequence
  Synthetic
VKLAGNSSLCPVSGWAPLSKDNSVRIGSKGDVFVIREPFISCSPLECRMFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTVGWSWPDGAELPFTIDK

SEQ ID NO: 23 - protein, 387 aa, Artificial Sequence
  Synthetic
VKLAGNSSLCPVSGWAPLSKNNAVRIGSKGDVFVIREPFISCSPLECRMFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTVGWSWPDGAELPFTIDK

SEQ ID NO: 24 - protein, 387 aa, Artificial Sequence
  Synthetic
VKLAGNSSLCPVSGWAPLAKDNSVRIGSKGDVFVIREPFISCSPLECRMFFLTQGALLNDKHSNGTIKDRSPYRTLMSVPLGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIIAFCGVNSDTVGWSWPDGAELPFTIDK

SEQ ID NO: 25 - protein, 387 aa, Artificial Sequence
  Synthetic
VKLAGNSSLCPVSGWAPLSKDNSVRIGSKGEIFVIREPFISCSPLECRTFFLTQGALLNDKHSNGTIKDRSPYRTLMSCPIGSPPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTTGWSWPDGAELPFTIDK

SEQ ID NO: 26 - protein, 387 aa, Artificial Sequence
  Synthetic
VKLAGNSSLCPVSGWAPLSKDNSVRIGSKGEVFVIREPFISCSPLECRQFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTTGWSWPDGAELPFTIDK

SEQ ID NO: 27 - protein, 387 aa, Artificial Sequence
  Synthetic
VKLAGNSSLCPVSGWAPLSKNNAVRIGSKGEVFVIREPFISCSPLECRQFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTTGWSWPDGAELPFTIDK

SEQ ID NO: 28 - protein, 387 aa, Artificial Sequence
  Synthetic
VKLAGNSSLCPVSGWAPLSKDNSVRIGSKGEIFVIREPFISCSPLECRQFFLTQGALLNDKHSNGTIKDRSPYRTLMSCPIGSPPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTTGWSWPDGAELPFTIDK

SEQ ID NO: 29 - protein, 387 aa, Artificial Sequence
  Synthetic
VKLAGNSSLCPVSGWAPLSKDNSVRIGSKGEVFVIREPFISCSPLECRQFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTTGWSWPDGAELPFTIDK

SEQ ID NO: 30 - protein, 387 aa, Artificial Sequence
  Synthetic
VKLAGNSSLCPVSGWAPLSKDNSVRIGSKGEVFVIREPFISCSPLECRMFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTTGWSWPDGAELPFTIDK

SEQ ID NO: 31 - protein, 387 aa, Artificial Sequence
  Synthetic
VKLAGNSSLCPVSGWAPLSKNNAVRIGSKGEVFVIREPFISCSPLECRMFFLTQGALLNDKHSNGTIKDRSPYRTLMSCPIGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIISFCGVNSDTTGWSWPDGAELPFTIDK

SEQ ID NO: 32 - protein, 387 aa, Artificial Sequence
  Synthetic
VKLAGNSSLCPVSGWAPLAKDNSVRIGSKGEVFVIREPFISCSPLECRMFFLTQGALLNDKHSNGTIKDRSPYRTLMSCPLGSVPSPTNSRFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIIAFCGVNSDTTGWSWPDGAELPFTIDK

SEQ ID NO: 33 - protein, 387 aa, Artificial Sequence
  Synthetic
EYRNWSKPQCNITGFAPFSKDNSIRLSAGGDIWVTREPYVSCDPDKCYQFALGQGTTLNNVHSNDTVHDRTPYRTLLMNELGQPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 34 - protein, 387 aa, Artificial Sequence
  Synthetic
EYRNWSKPQCNITGFAPFSKDNSIRLSAGGDIWVTREPYVSCDPDKCYQFALGQGTTLNNVHSNDTVHDRTPYRTLLMNELGQPFHLGTKQVCVAWSSSSCHDGKAWLHVCVSGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 35 - protein, 390 aa, Artificial Sequence
  Synthetic
HFMNNTEALCDAKGFAPFSKDNGIRIGSRGHVFVIREPFVSCSPTECRTFFLTQGSLLNDKHSNGTVKDRSPYRTLMSVEIGSSPNVYQARFEAVAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCKDNWTGTNRPVLVISPDLSYRVGYLCAGLPSDTPRGEDSQFTGSCTSPMGNQGYGVKGFGFRQGNDVWMGRTISRTSRSGFEILKVRNGWVQNSKEQIKRQVVVDNLNWSGYSGSFTLPAELTKRNCLVPCFWVEMIRGNPEEKTIWTSSSSIVMCGVDHEIADWSWHDGAILPFDIDKM

SEQ ID NO: 36 - protein, 390 aa, Artificial Sequence
  Synthetic
HFMNNTEALCDAKGFAPFSKDNGIRIGSRGHVFVIREPFVSCSPTECRTFFLTQGSLLNDKHSNGTVKDRSPYRTLMSVPIGSSPNVYQARFEAVAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCKDNWTGTNRPVLVISPDLSYRVGYLCAGLPSDTPRGEDSQFTGSCTSPMGNQGYGVKGFGFRQGNDVWMGRTISRTSRSGFEILKVRNGWVQNSKEQIKRQVVVDNLNWSGYSGSFTLPAELTKRNCLVPCFWVEMIRGNPEEKTIWTSSSSIVMCGVDHEIADWSWHDGAILPFDIDKM

SEQ ID NO: 37 - protein, 390 aa, Artificial Sequence
  Synthetic
HFMNNTEALCDAKGFAPFSKDNGIRIGSRGHVFVIREPFVSCSPTECRTFFLTQGSLLNDKHSNGTVKDRSPYRTLMSVPIGSSPNVYQARFEAIAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCKDNWTGTNRPVLVISPDLSYRVGYLCAGLPSDTPRGEDSQFTGSCTSPMGNQGYGVKGFGFRQGNDVWMGRTISRTSRSGFEILKVRNGWVQNSKEQIKRQVVVDNLNWSGYSGSFTLPAELTKRNCLVPCFWVEMIRGNPEEKTIWTSSSSIVMCGVDHEIADWSWHDGAILPFDIDKM

SEQ ID NO: 38 - protein, 390 aa, Artificial Sequence
  Synthetic
HFMNNTEALCDAKGFAPFSKDNGIRIGSRGHVFVIREPFVSCSPTECRTFFLTQGSLLNDKHSNGTVKDRSPYRTLMSVPIGSSPNVYQARFEAIAWSATACHDGKKWMTIGVTGPDAKAVAIVHYGGIPTDVINSWAGDILRTQESSCTCIQGECFWVMTDGPANRQAQYRAFKAKQGKIVGQAEISFNGGHIEECSCYPNEGKVECVCKDNWTGTNRPVLVISPDLSYRVGYLCAGLPSDTPRGEDSQFTGSCTSPMGNQGYGVKGFGFRQGNDVWMGRTISRTSRSGFEILKVRNGWVQNSKEQIKRQVVVDNLNWSGYSGSFTLPAELTKRNCLVPCFWVEMIRGNPEEKTIWTSSSSIVMCGVDHEIADWSWHDGAILPFDIDKM

SEQ ID NO: 39 - protein, 387 aa, Artificial Sequence
  Synthetic
VILTGNSSLCPISGWAPLAKDNSIRIGSKGDVFVIREPFISCSHLECRMFFLTQGALLNDKHSNGTVKDRSPYRTLMSVPLGSVPSPYNARFESIAWSASACHDGMGWLTIGITGPDNGAVAILKYNGIITDTIKSWRNNILRTQESECACVNGSCFTIMTDGPSNGQASYKILKIEKGKVTKSIELNAPNYHYEECSCYPDTGKVMCVCRDNWHGSNRPWVSFDQNLDYQIGYICSGVFGDNPRPNDGTGSCGPVSSNGANGIKGFSFRYDNGVWIGRTKSTSSRSGFEMIWDPNGWTETDSSFSVRQDIVAITDWSGYSGSFVMHPELTGLDCMVPCFWVELIRGQPKENTIWTSGSIIAFCGVNSDTVGWSWPDGAELPFSIDK

SEQ ID NO: 40 - protein, 387 aa, Artificial Sequence
  Synthetic
VKLAGNSSLCPINGWAPLSKDNSVRIGSKGDVFVIREPFISCSHLECRQFFLTQGALLNDKHSNGTVKDRSPHRTLMSVPIGSVPSPYNARFESIAWSASACHDGTSWLTIGITGPDNGAVAILKYNGIITDTIKSWRNNILRTQESECACVNGSCFTVMTDGPSNGQASYKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVMHPELTGLDCIVPCFWVELIRGRPKESTIWTSGSSISFCGVNSDTVGWSWPDGAELPFTIDK

SEQ ID NO: 41 - protein, 387 aa, Artificial Sequence
  Synthetic
VKLAGNSSLCPINGWAPLSKDNSVRIGSKGDIFVIREPFISCSHLECRQFFLTQGALLNDKHSNGTVKDRSPHRTLMSVPIGSPPSPYNARFESIAWSASACHDGTSWLTIGITGPDNGAVAILKYNGIITDTIKSWRNNILRTQESECACVNGSCFTVMTDGPSNGQASYKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVMHPELTGLDCIVPCFWVELIRGRPKESTIWTSGSSISFCGVNSDTVGWSWPDGAELPFTIDK

SEQ ID NO: 42 - protein, 387 aa, Artificial Sequence
  Synthetic
VKLAGNSSLCPINGWAPLSKDNSVRIGSKGDIFVIREPFISCSPLECRQFFLTQGALLNDKHSNGTVKDRSPHRTLMSVPIGSPPSPYNARFESIAWSASACHDGTSWLTIGITGPDNGAVAILKYNGIITDTIKSWRNNILRTQESECACVNGSCFTVMTDGPSNGQASYKIFKIEKGKIVKSVEMDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVMHPELTGLDCIVPCFWVELIRGRPKESTIWTSGSSISFCGVNSDTVGWSWPDGAELPFTIDK

SEQ ID NO: 43 - protein, 387 aa, Artificial Sequence
  Synthetic
VILTGNSSLCPIRGWAPLSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAILKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSIISFCGVNSDTVDWSWPDGAELPFTIDK

SEQ ID NO: 44 - protein, 387 aa, Artificial Sequence
  Synthetic
VILTGNSSLCPIRGWAPLSKDNSVRIGSKGDVFVIREPFISCSHLECRQFFLTQGALLNDKHSRGTFKDRSPYRTLMSVPIGSVPSPYNARFESIAWSASACHDGMGWLTIGITGPDDGAVAILKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSIISFCGVNSDTVDWSWPDGAELPFTIDK

SEQ ID NO: 45 - protein, 387 aa, Artificial Sequence
  Synthetic
VILTGNSSLCPIRGWAPLSKDNSVRIGSKGDVFVIREPFISCSHLECRQFFLTQGALLNDKHSRGTFKDRSPYRTLMSVPIGSVPSPYNARFESIAWSASACHDGMGWLTIGITGPDDGAVAILKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSSISFCGVNSDTVDWSWPDGAELPFTIDK

SEQ ID NO: 46 - protein, 387 aa, Artificial Sequence
  Synthetic
VILTGNSSLCPIRGWAPLSKDNSVRIGSKGDIFVIREPFISCSHLECRQFFLTQGALLNDKHSRGTFKDRSPYRTLMSVPIGSPPSPYNARFESIAWSASACHDGMGWLTIGITGPDDGAVAILKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSSISFCGVNSDTVDWSWPDGAELPFTIDK

SEQ ID NO: 47 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 48 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 49 - protein, 387 aa, Artificial Sequence
  Synthetic
VKLAGNSSLCPVSGWAILSKDNSVRIGSKGDVFVIREPFISCSPLECRTFFLTQGALLNDKHSNGTIKDRSPYRTLMSVPIGSVPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 50 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSVPIGSVPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 51 - protein, 387 aa, Artificial Sequence
  Synthetic
VKLAGNSSLCPVSGWAIYSKDNSVRIGSKGDIFVIREPFISCSPLECRTFFLTQGALLNDKHSNGTIKDRSPYRTLMSVPIGSPPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 52 - protein, 387 aa, Artificial Sequence
  Synthetic
VKLAGNSSLCPVSGWAIYSKDNSVRIGSKGDVFVIREPFISCSPLECRQFFLTQGALLNDKHSNGTIKDRSPYRTLMSVPIGSVPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 53 - protein, 387 aa, Artificial Sequence
  Synthetic
VKLAGNSSLCPVSGWAIYSKDNSVRIGSKGDIFVIREPFISCSPLECRQFFLTQGALLNDKHSNGTIKDRSPYRTLMSVPIGSPPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 54 - protein, 387 aa, Artificial Sequence
  Synthetic
VKLAGNSSLCPVSGWAILAKDNSVRIGSKGDVFVIREPFISCSPLECRMFFLTQGALLNDKHSNGTIKDRSPYRTLMSVPLGSVPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSIIAFCGVNSDTVGWSWPDGAELPFTIDK

SEQ ID NO: 55 - protein, 387 aa, Artificial Sequence
  Synthetic
VKLAGNSSLCPVSGWAILAKDNSVRIGSKGDIFVIREPFISCSPLECRMFFLTQGALLNDKHSNGTIKDRSPYRTLMSVPLGSPPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSIIAFCGVNSDTVGWSWPDGAELPFTIDK

SEQ ID NO: 56 - protein, 387 aa, Artificial Sequence
  Synthetic
VKLAGNSSLCPVSGWAPLSKDNSVRIGSKGDIFVIREPFISCSPLECRTFFLTQGALLNDKHSNGTIKDRSPYRTLMSVPIGSPPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 57 - protein, 387 aa, Artificial Sequence
  Synthetic
VKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 58 - protein, 387 aa, Artificial Sequence
  Synthetic
VKLAGNSSLCPVSGWAPLSKDNSVRIGSKGDIFVIREPFISCSPLECRQFFLTQGALLNDKHSNGTIKDRSPYRTLMSVPIGSPPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 59 - protein, 387 aa, Artificial Sequence
  Synthetic
VKLAGNSSLCPVSGWAPLAKDNSVRIGSKGDVFVIREPFISCSPLECRMFFLTQGALLNDKHSNGTIKDRSPYRTLMSVPLGSVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIAFCGVNSDTVGWSWPDGAELPFTIDK

SEQ ID NO: 60 - protein, 387 aa, Artificial Sequence
  Synthetic
VKLAGNSSLCPVSGWAPLAKDNSVRIGSKGDIFVIREPFISCSPLECRMFFLTQGALLNDKHSNGTIKDRSPYRTLMSVPLGSPPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIAFCGVNSDTVGWSWPDGAELPFTIDK

SEQ ID NO: 61 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSCPIGSVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 62 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSCPIGTVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 63 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSCPIGVVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 64 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSCPIGAVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 65 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSCPIGIVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 66 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGTVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 67 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGVVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 68 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGAVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 69 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGIVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 70 - protein, 387 aa, Artificial Sequence
  Synthetic
VKLAGNSSLCPVSGWAIYSKDNSVRIGSKGDVFVIREPFISCSPLECRTFFLTQGALLNDKHSNGTIKDRSPYRTLMSVPIGEVPSPYNARFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSYKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 71 - protein, 387 aa, Artificial Sequence
  Synthetic
VKLAGNSSLCPVSGWAPLCKDNSVRIGSKGDVFVIREPFVSCSPLECRTFFLTQGALLNDKHSNGTIKDRSPYRTLMSVPICSVPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSIVFCGVNSDTVGWSWPDGAELPFTIDK

SEQ ID NO: 72 - protein, 387 aa, Artificial Sequence
  Synthetic
VKLAGNSSLCPVSGWAPLCKDNSVRIGSKGDIFVIREPFVSCSPLECRTFFLTQGALLNDKHSNGTIKDRSPYRTLMSVPICSPPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSIVFCGVNSDTVGWSWPDGAELPFTIDK

SEQ ID NO: 73 - protein, 387 aa, Artificial Sequence
  Synthetic
VKLAGNSSLCPVSGWAPLCKDNSVRIGSKGDVFVIREPFVSCSPLECRQFFLTQGALLNDKHSNGTIKDRSPYRTLMSVPICSVPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSIVFCGVNSDTVGWSWPDGAELPFTIDK

SEQ ID NO: 74 - protein, 387 aa, Artificial Sequence
  Synthetic
VKLAGNSSLCPVSGWAPLCKDNSVRIGSKGDIFVIREPFVSCSPLECRQFFLTQGALLNDKHSNGTIKDRSPYRTLMSVPICSPPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSIVFCGVNSDTVGWSWPDGAELPFTIDK

SEQ ID NO: 75 - protein, 387 aa, Artificial Sequence
  Synthetic
VKLAGNSSLCPVSGWAPLCKDNSVRIGSKGDVFVIREPFISCSPLECRMFFLTQGALLNDKHSNGTIKDRSPYRTLMSVPLCSVPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIAFCGVNSDTVGWSWPDGAELPFTIDK

SEQ ID NO: 76 - protein, 387 aa, Artificial Sequence
  Synthetic
VKLAGNSSLCPVSGWAPLCKDNSVRIGSKGDVFVIREPFVSCSPLECRMFFLTQGALLNDKHSNGTIKDRSPYRTLMSVPICSVPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIVFCGVNSDTVGWSWPDGAELPFTIDK

SEQ ID NO: 77 - protein, 387 aa, Artificial Sequence
  Synthetic
VKLAGNSSLCPVSGWAPLCKDNSVRIGSKGDIFVIREPFISCSPLECRMFFLTQGALLNDKHSNGTIKDRSPYRTLMSVPLCSPPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIAFCGVNSDTVGWSWPDGAELPFTIDK

SEQ ID NO: 78 - protein, 387 aa, Artificial Sequence
  Synthetic
VKLAGNSSLCPVSGWAPLCKDNSVRIGSKGDIFVIREPFVSCSPLECRMFFLTQGALLNDKHSNGTIKDRSPYRTLMSVPICSPPSPYNARVESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIVFCGVNSDTVGWSWPDGAELPFTIDK

SEQ ID NO: 79 - protein, 387 aa, Artificial Sequence
  Synthetic
VKLAGNSSLCPVSGWAPLSKDNSVRIGSKGEVFVIREPFISCSPLECRTFFLTQGALLNDKHSNGTIKDRSPYRTLMSCPIGSVPSPSNSRFESVAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTTGWSWPDGAELPFTIDK

SEQ ID NO: 80 - protein, 387 aa, Artificial Sequence
  Synthetic
VKLAGNSSLCPVSGWAPYSKDNSVRIGSKGEVFVIREPFISCSPLECRTFFLTQGALLNDKHSNGTIKDRSPYRTLMSCPIGSVPSPSNSRFESVAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTTGWSWPDGAELPFTIDK

SEQ ID NO: 81 - protein, 387 aa, Artificial Sequence
  Synthetic
VKLAGNSSLCPVSGWAPLSKDNSVRIGSKGEVFVIREPFISCSPLECRTFFLTQGALLNDKHSNGTIKDRSPYRTLMSCPIGSVPSPTNSRFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTTGWSWPDGAELPFTIDK

SEQ ID NO: 82 - protein, 387 aa, Artificial Sequence
  Synthetic
VKLAGNSSLCPVSGWAPLSKDNSVRIGSKGEVFVIREPFISCSPLECRTFFLTQGALLNDKHSNGTIKDRSPYRTLMSCPIGSVPSPSNSRFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTTGWSWPDGAELPFTIDK

SEQ ID NO: 83 - protein, 387 aa, Artificial Sequence
  Synthetic
VKLAGNSSLCPVSGWAPYSKDNSVRIGSKGEVFVIREPFISCSPLECRTFFLTQGALLNDKHSNGTIKDRSPYRTLMSVPIGSVPSPTNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTTGWSWPDGAELPFTIDK

SEQ ID NO: 84 - protein, 387 aa, Artificial Sequence
  Synthetic
VKLAGNSSLCPVSGWAPLSKDNSVRIGSKGEVFVIREPFISCSPLECRTFFLTQGALLNDKHSNGTIKDRSPYRTLMSVPIGSVPSPTNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 85 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTTGWSWPDGAELPFTIDK

SEQ ID NO: 86 - protein, 387 aa, Artificial Sequence
  Synthetic
VKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAILKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTTGWSWPDGAELPFTIDK

SEQ ID NO: 87 - protein, 387 aa, Artificial Sequence; identical to SEQ ID NO: 47
  Synthetic

SEQ ID NO: 88 - protein, 387 aa, Artificial Sequence
  Synthetic
VKLAGNSSLCPVSGWAPLSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 89 - protein, 387 aa, Artificial Sequence
  Synthetic
VKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 90 - protein, 387 aa, Artificial Sequence
  Synthetic
VKLAGNSSLCPVSGWAPLAKDNSVRIGSKGDVFVIREPFISCSPLECRMFFLTQGALLNDKHSNGTIKDRSPYRTLMSVPLGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSIIAFCGVNSDTVGWSWPDGAELPFTIDK

SEQ ID NO: 91 - protein, 387 aa, Artificial Sequence
  Synthetic
VKLAGNSSLCPINGWAPLSKDNSVRIGSKGDVFVIREPFISCSHLECRQFFLTQGALLNDKHSNGTVKDRSPHRTLMSVPIGSVPSPYNARFESIAWSASACHDGTSWLTIGITGPDNGAVAVLKYNGIITDTIKSWRNNILRTQESECACVNGSCFTVMTDGPSNGQASYKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVMHPELTGLDCIVPCFWVELIRGRPKESTIWTSGSSISFCGVNSDTVGWSWPDGAELPFTIDK

SEQ ID NO: 92 - protein, 387 aa, Artificial Sequence
  Synthetic
VILTGNSSLCPIRGWAPLSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSIISFCGVNSDTVDWSWPDGAELPFTIDK

SEQ ID NO: 93 - protein, 387 aa, Artificial Sequence
  Synthetic
VILTGNSSLCPIRGWAPLSKDNSVRIGSKGDVFVIREPFISCSHLECRQFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVMHPELTGLDCMVPCFWVELIRGLPEEDAIWTSGSIISFCGVNSDTVDWSWPDGAELPFTIDK

SEQ ID NO: 94 - protein, 387 aa, Artificial Sequence
  Synthetic
VKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSSISFCGVNSDTVGWSWPDGAELPFTIDK

SEQ ID NO: 95 - protein, 387 aa, Artificial Sequence
  Synthetic
VKLAGNSSLCPVSGWAPLAKDNSVRIGSKGDVFVIREPFISCSPLECRMFFLTQGALLNDKHSNGTIKDRSPYRTLMSVPLGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVMHPELTGLDCIVPCFWVELIRGRPKENTIWTSGSIIAFCGVNSDTVGWSWPDGAELPFTIDK

SEQ ID NO: 96 - protein, 353 aa, Artificial Sequence
  Synthetic
VLAGNSSLCPSGWAIYSKDNRIGSKGDVFVIREPFISCSLECRTFFLTQGALLNDKHSNGTKDRSPYRTLMSCPGEPSPYNSRFESVAWSASACHDGIWLTIGISGPDNGAVAVLKYNGIITDTIKSWRNNILRTQESECACVNGSCFTVMTDGPSNGQASYKIFIEKGKVKSVENAPNYHYEECSCYPDVCRDNWHGSNRPWVSFNQNLEYQIGYICSGFGDNPRPNDTGSCGPSSNGAGVKGFSFKYGNGVWIGRTKSSSRGFEMIWDPNGWTTDFSKQDIVIWSGYSGSFVQHPELTGLDCRPCFWVELIRGRPKTIWTSGSSISFCGVNSDTVGWSWPDGAELPFTIDK

SEQ ID NO: 97 - protein, 382 aa, Artificial Sequence
  Synthetic
VTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGVCRDNWHGSNRPWVSFNQNLEYQIGYICSGVFGDNPRPNDGTGSCGPMSSNGAYGVKGFSFKYGNGVWIGRTKSTSSRSGFEMIWDPNGWTETDSSFSMKQDIVAITDWSGYSGSFVQHPELTGLDCMRPCFWVELIRGRPKDTIWTSGSSISFCGVNSDTVGWSWPDGAELPFTIDK

SEQ ID NO: 98 - protein, 324 aa, Artificial Sequence
  Synthetic
VLAGNSSLCSGWAIYKDNSRIGSKGDVFVIREPFISCSLECRTFFLTQGALLNDKHSNGTKRSPYRLMSCPGEPSPYNSFESVAWSASACHDGWLTIGISGPDNGAVAVLKYNGIITTIKSWILRTQESECCNGSCFTMTDGPSNASYKIFIEKGKKSENAPNYEECSCYPDCVCRDNWHGSNRPWVSFNQNLYQIGYICSGFGDNPRPDGSCPVGANGVKGFSKYNGVWIGRTKSRGFEMIWDPNGWTTDFSKQDVIWSGYSGSFVQHPELTGLDCIRPCFWVELRGPENTIWTSGSSISFCGVNSDTWSWPDGAELPFTIDK

SEQ ID NO: 99 - protein, 388 aa, Artificial Sequence
  Synthetic
VTLAGNSSLCSISGWAIYTKDNSIRIGSKGDVFVIREPFISCSHLECRTFFLTQGALLNDKHSNGTVKERSPYRALMSCPLGEAPSPYNSKFESVAWSASACHDGMGWLTIGISGPDNGAVAVLKYNGIITGTIKSWKKQILRTQESECVCMNGSCFTIMTDGPSNKAASYKIFKIEKGKVTKSIELNAPNFYYEECSCYPDTGIVMCVCRDNWHGSNRPWVSFNQNLDYQIGYICSGVFGDNPRPEDGEGSCNPVTVDGANGVKGFSYKYDNGVWIGRTKSNRLRKGFEMIWDPNGWTNTDSDFSVKQDVVAITDWSGYSGSFVQHPELTGLDCIRPCFWVELVRGLPRENTTIWTSGSSISFCGVNSDTANWSWPDGAELPFTIDK

(19) United States
(12) Patent Application Publication
(10) Pub. No.: US 2023/0090779 A1
(43) Pub. Date: Mar. 23, 2023

(54) ENGINEERED INFLUENZA NEURAMINIDASE ANTIGENS

(71) Applicants: University of Washington, Seattle, WA (US); THE USA,AS REPRESENTED BY THE SECRETARY, DEPARTMENT OF HEALTH AND HUMAN SERVICES, Bethesda, MD (US)

(72) Inventors: Neil P. KING, Seattle, WA (US); Daniel ELLIS, Seattle, WA (US); Masaru KANEKIYO, Seattle, WA (US); Julia LEDERHOFER, Seattle, WA (US); Barney S. GRAHAM, Seattle, WA (US)

(21) Appl. No.: 17/904,157

(22) PCT Filed: Mar. 4, 2021
(86) PCT No.: PCT/US2021/020804

§ 371 Date: Aug. 12, 2022

Related U.S. Application Data
(60) Provisional application No. 62/986,295, filed on Mar. 6, 2020.

(51) Int. Cl. C12N 9/24; A61K 39/145; A61P 31/16

(52) U.S. Cl. CPC C12N 9/2402; C12Y 302/01018; A61K 39/145; A61P 31/16; C12N 2760/16022; C07K 2319/00

(57) ABSTRACT

The disclosure provides non-naturally occurring mutant neuraminidase (NA) polypeptides that improve expression and or modifies the open/closed tetrameric conformational state of the NA polypeptide, and uses thereof.

### CROSS REFERENCE

This application claims priority to U.S. Provisional Patent Application Ser. No. 62/986,295 filed Mar. 6, 2020, incorporated by reference herein in its entirety.

### BACKGROUND

Influenza viruses claim countless lives and pose a significant public health and economic burden globally every year. The development of highly effective vaccines to ever-evolving influenza viruses has been a major public health priority. There are two major viral glycoproteins on the influenza virion, the hemagglutinin (HA) and the neuraminidase (NA), which facilitate viral entry and egress from hast cells, respectively. NA is a homotetrameric, type II integral membrane protein, the N terminus of which is oriented towards the interior of the virus. The C-terminal globular head domain of NA contains six topologically identical beta sheets arranged in a propeller-like structure, comprises the discontinuous catalytic site, and is supported by a stalk domain. NA cleaves terminal sialic acid from glycans on the host cell surface, thereby releasing nascent viral particles. Additionally, it is believed that NA might play a role in viral entry. These characteristics make NA an attractive target as a vaccine immunogen, yet NA has historically been neglected in vaccine development.

### SUMMARY

In one aspect, the disclosure provides non-naturally occurring mutant neuraminidase (NA) polypeptides that improve expression and/or modifies the open-closed tetrameric conformational state of the NA polypeptide. In one embodiment, wherein the polypeptide is (a) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 1, 2, 3, 4, 5, 6, or all 7 of the following amino acid residues relative to SEQ ID NO:1 when aligned by protocol 1 or protocol 2:
161T/A, 105A, 165T/I, 166P, 196Q, 203Y, 444V; or

(b) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 2, 3, 4, 5, 6, 7, 8, 9, 10, 11, 12, 13, 14, 15, 16, or all 17 of the following amino acid residues relative to SEQ ID NO:1 when aligned by protocol 1 or protocol 2:
161T/A/V/S/T, 100L, 408M, 419V, 99P, 103N, 105A, 131Q/M, 163I/L, 165T/S/V/A/I, 166P, 177I, 196Q/T, 203Y, 205I, 442I, 444V.

In another embodiment, the polypeptide is

(a) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 1, 2, 3, 4, 5, 6, 7, or all 8 of the following amino acid residues relative to SEQ ID NO:2 when aligned by protocol 1 or protocol 2:
101C, 131M, 162P, 165S/T, 166V, 195Q, 202Y, 443S; or

(b) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 2, 3, 4, 5, 6, 7, 8, 9, 10, 11, or all 12 of the following amino acid residues relative to SEQ ID NO:2 when aligned by protocol 1 or protocol 2:
101C/A, 103N, 105A, 106V, 131M, 162P, 163I, 165S/T/A/I, 166V, 195Q, 202Y, 443S.

In another embodiment, the polypeptide is

(a) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 1, 2, 3, 4, 5, 6, 7, 8, 9, 10, or all 11 of the following amino acid residues relative to SEQ ID NO: 3 when aligned by protocol 1 or protocol 2:
103N, 105S, 131Q/M, 157T, 165S/I, 166P, 196Q, 203Y, 205I, 443S, 445V; or

(b) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 2, 3, 4, 5, 6, 7, 8, 9, 10, 11, 12, or all 13 of the following amino acid residues relative to SEQ ID NO:1 when aligned by protocol 1 or protocol 2:
103N, 105S, 106V, 131Q/M, 157T, 163L, 165S/I/V/A, 166P/V, 196Q, 203Y, 205I, 443S, 445V.

In a further embodiment, the polypeptide is

(a) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 1, 2, 3, 4, 5, 6, 7, 8, 9, 10, 11, 12, 13, 14, 15, or all 16 of the following amino acid residues relative to SEQ ID NO:4 when aligned by protocol 1 or protocol 2:
160V/S/T/A, 409M, 102N, 104S/A, 105V, 112D, 130Q/M, 162L, 164S/T/A/I, 165P, 176I, 195Q, 202Y, 204I, 443I, 445V; or

(b) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 2, 3, 4, 5, 6, 7, 8, 9, 10, 11, 12, 13, 14, 15, or all 16 of the following amino acid residues relative to SEQ ID NO:4 when aligned by protocol 1 or protocol 2:
160V/S/T/A, 409M, 102N, 104S/A, 105V, 112D, 130Q/M, 162L, 164S/T/A/I, 165P/V, 176I, 195Q, 202Y, 204I, 443I, 445V.

In one embodiment, the polypeptide is

(a) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, 2, 3, 4, 5, 6, 7, 8, 9, 10, 11, 12, or all 13 of the following amino acid residues relative to SEQ ID NO:5 when aligned by protocol 1 or protocol 2:
97L, 410M, 96P, 98A, 100N, 102S/A, 110D, 128Q/M, 160I, 162V/A/I, 163V/P, 193Q/T, 445I; or

(b) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 2, 3, 4, 5, 6, 7, 8, 9, 10, 11, 12, 13, 14, 15, or all 16 of the following amino acid residues relative to SEQ ID NO:5 when aligned by protocol 1 or protocol 2:
97L, 410M, 96P, 98A, 100N, 102S/A, 103V, 110D, 128Q/M, 154T, 160I, 162V/A/I/T, 163V/P, 174I, 193Q/T, 445I.

In a further embodiment, the polypeptide is

(a) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 1, 2, 3, 4, 5, 6, 7, 8, 9, 10, or all 11 of the following amino acid residues relative to SEQ ID NO:6 when aligned by protocol 1 or protocol 2:
99P, 103N, 113D, 131Q, 161I, 162P, 163I, 165S/T/V/A, 196Q, 203Y, 445S; or

(b) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 2, 3, 4, 5, 6, 7, 8, 9, 10, 11, 12, 13, 14, 15, or all 16 of the following amino acid residues relative to SEQ ID NO:6 when aligned by protocol 1 or protocol 2:
99 P, 103N, 105S, 106V, 113D, 131Q, 157T, 161I, 162P, 163I/L, 165S/T/V/A/I, 166V/P, 196Q, 203Y, 445S.

In one embodiment, the polypeptide is

(a) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 1, 2, 3, 4, 5, 6, 7, 8, 9, 10, or all 11 of the following amino acid residues relative to SEQ ID NO:7 when aliened by protocol 1 or protocol 2:
98P, 102N, 104S, 112D, 130Q, 156T, 162I, 164V/A/I, 165V, 202Y, 448V; or

(b) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 2, 3, 4, 5, 6, 7, 8, 9, 10, 11, 12, or all 13 of the following amino acid residues relative to SEQ ID NO:7 when aligned by protocol 1 or protocol 2:
98P, 102N, 104S, 112D, 130Q, 156T, 162I, 164V/A/I, 165V, 176I, 202Y, 446I, 448V.

In another embodiment, the polypeptide is

(a) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 1, 2, 3, 4, 5, 6, 7, 8, 9, 10, or all 11 of the following amino acid residues relative to SEQ ID NO:8 when aligned by protocol 1 or protocol 2:
408M, 101N, 103A/S, 111D, 129Q, 161P/L, 163S/T/V/A/I, 164V, 194Q, 201Y, 442I; or

(b) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 2, 3, 4, 5, 6, 7, 8, 9, 10, 11, 12, 13, 14, or all 15 of the following amino acid residues relative to SEQ ID NO:8 when aligned by protocol 1 or protocol 2:
98L, 408M, 101N, 103A/S, 104V, 111D, 129Q/M, 161P/L, 163S/T/V/A/I/S/T, 164V/P, 175I, 194Q, 201Y, 203I, 442I.

In one embodiment, the polypeptide is

(a) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 1, 2, 3, 4, 5, 6, 7, 8, 9, or all 10 of the following amino acid residues relative to SEQ ID NO:9 when aligned by protocol 1 or protocol 2:
94P, 95L, 100S, 126Q/M, 160V/A/I, 161V, 191Q, 198Y, 439S, 441V; or

(b) at least 75%, 80%, 85%, 90%, 91%, 92%, 93%, 94%, 95%, 96%, 97%, 98%, or 99% identical to the N9 NA polypeptide of SEQ ID NO:9, and wherein live non-naturally occurring polypeptide includes 2, 3, 4, 5, 6, 7, 8, 9, 10, 11, 12, 13, or all 14 of the following amino acid residues relative to SEQ ID NO:9 when aligned by protocol 1 or protocol 2:
94P, 95L, 98M, 100S/A, 126Q/M, 152T, 158L 160V/A/I/T, 161V, 191Q, 198Y, 200I, 439S, 441V.

In one embodiment, the polypeptide is at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes a 160Q amino acid mutation relative to SEQ ID NO:9 when aligned by protocol 1, or may include a combination of 160Q/E and 172V amino acid residues relative to SEQ ID NO:9 when aligned by protocol 1 or protocol 2.

In a further embodiment, the disclosure provides compositions, comprising one or more of the polypeptides of any embodiment linked to a scaffold. In one embodiment, the scaffold comprises a protein scaffold. In a further embodiment, the polypeptide is covalently linked to a protein subunit of the protein scaffold to form a fusion protein.

In various further aspects, the disclosure provides nucleic acids encoding polypeptides or fusion proteins of the disclosure, expression vector comprising the nucleic acids of the disclosure operatively linked to a suitable control sequence; host cells comprising nucleic acids, expression vectors, and/or polypeptide or fusion proteins of the disclosure, and pharmaceutical compositions or vaccines, comprising

(a) one or more of the polypeptides, composition, nucleic acid, expression vector, and/or the host cell of the disclosure; and

(b) a pharmaceutically acceptable carrier.

In another aspect, the disclosure provides methods for treating or limiting development of an influenza infection, comprising administering to a subject in need thereof an amount effective to treat or limit development of the influenza infection of a polypeptide, fusion protein, composition, vaccine, nucleic acid, expression vector, host cell, pharmaceutical composition, and/or vaccine of any preceding claim.

### DESCRIPTION OF THE FIGURES

FIG. 1(A-B). Overview of Influenza Neuraminidase design and expression of soluble NA protein, (A) Schematic representation (H1N1 California 09pdm) of (1) wild type (WT) and two recombinant NA designs (2 and 3), that vary in amino acid length. The stalk domain of the WT was replaced by a 6× His-tag, a hVSAP domain for protein tetramerization (TD), a thrombin site and a GG linker, (B) Gel filtration chromatograms of His-purified H1N1 California 09pdm recorded at 280 nm wavelength.

FIG. 2. Characterization of NA subtypes. Different subtype NAs differentially adapt to open and closed tetramers: A/California/07/2009 H1N1, A/New Caledonia/20/1999 H1N1, A/Michigan 45/2015 H1N1, A/WSN/1933 H1N1 and A/Sichuan/26221/2014 H5N6 are open. A/Netherlands/219/2003 H7N7, A/Jiangxi-Donghu/346-2/2013 H10N8 and A/Anhui/1/2013 H7N9 are mixed open/closed. A/Wisconsin/67/2005 H3N2, A/Swine/Missouri/2124514/2006 H2N3, A/Red knot/Delaware Bay/310-2016 H10N4 and A/Shorebird/Delaware Bay/309/2016 H10N5 are closed.

FIG. 3. Design process of A/California/07/2009 H1N1 N1 neuraminidase, from loose to tight tetramers. Design 94 (SEQ ID NO:79) with ten different mutations resulted in 45% tight tetramer particles. Design 113 (SEQ ID NO:81) with 11 different mutations resulted in 80-90% tight tetramer particles. Design 155 (SEQ ID NO: 13) with ten different mutations resulted in 90-100% tight tetramer particles.

FIG. 4. Design process of A/Michigan/45/2015 H1N1 N1 neuraminidase, from loose to tight tetramers based on A/California/07/2009 H1N1 N1 design. Design 300 (SEQ ID NO:14) with ten mutations based on design 155 resulted in 75% tight tetramer particles. Design 174 (SEQ ID NO: 18) with one additional mutation resulted in 100% tight tetramer particles. Three other distinct solutions for stabilizing mutations were found as well.

FIG. 5. Design process of A/WSN/1933 H1N1 N1 neuraminidase, from loose to tight tetramers based on A/California/07/2009 H1N1 N1 design c155 Design 366 (SEQ ID NO: 14) with 16 different mutations resulted in 80% light tetramer particles. Ten of those mutation are the same mutations as in design e155 (SEQ ID NO: 13).

FIG. 6. Design process of A/Vietnam/1203/04 H5N1 N1 neuraminidase, from loose to tight tetramers based on A/California/07/2009 H1N1 N1 design cI55. Design 348 resembles same mutations as design 155. Design 354 (SEQ ID NO:40) with 14 different mutations resulted in 100% tight tetramer particles. Ten of those mutation are the same mutations as in design c155.

FIG. 7. Design process of A/Jiangxi-Donghu/346-2/2013 H10N8 N8 neuraminidase, from loose to tight tetramers based on A/California/07/2009 H1N1 N1 design. Design 285 (SEQ ID NO:36) with two space D mutations resulted in 100% tight tetramer particles.

FIG. 8. Design process of A/Wisconsin/67/2005 H3N2 N2 neuraminidase, to destabilize the tight tetrameric particles based on A/California/07/2009 H1N1 N1 design. Introducing two mutations in design 157 did not result in an open construct. Adding one additional mutation 165Q resulted in 0% tight tetramer particles with some unassembled subunits.

FIG. 9. BLASTp alignment of SEQ ID NO:1 with other N1 strain sequences that contain deletions or insertions. BLASTp alignment allows for residue positions of reference strains to be matched with corresponding residue positions in strains from the same subtype, even when sequences contain arbitrary numbers of insertions and deletions relative to the reference strain.

### DETAILED DESCRIPTION

All references cited are herein incorporated by reference in their entirety. Within this application, unless otherwise stated, the techniques utilized may be found in any of several well-known references such as: Molecular Cloning: A laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows, alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Scr; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in air inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The disclosure provides non-naturally occurring mutant neuraminidase (NA) polypeptides that improves expression and or modifies the open/closed tetrameric conformational state of the NA polypeptide. As detailed in the examples that follow, the inventors have produced recombinant NA polypeptides in which head domains comprising stabilizing mutations are connected to tetramerization domains. We initially found that many wild-type sequences of beta propeller head domains from certain NA subtypes adopted “open” conformations in which the head domains extended individually off of the stalk-like tetramerization domain, without forming the crystallographically observed “closed” tetramer. Constructs comprising the head domains from other NA subtypes formed closed tetramers naturally. Similar constructs from yet other subtypes formed mixtures of open and closed tetramers. We identified specific mutations at multiple locations in NA sequences that dictate the open or closed conformational state of NA tetramers, and used these mutations to generate closed, stabilized tetramers from multiple NA subtypes. We also converted a naturally closed NA tetramer to a fully open conformation by substituting residues that we identified as pivotal for tetramer closure, confirming the importance of these residues for determining the conformational state of NA. Monoclonal antibodies (mAbs) that bind across the interface of two neighboring protomers in the closed configuration bind better to closed tetramers than open tetramers. The NA polypeptides of the disclosure are improved vaccine antigens in either soluble form or when presented on scaffolds.

In a first aspect, the NA polypeptides are:

(a) at least 75%, 80%, 85%, 90%, 91%, 92%, 93%, 94%, 95%, 96%, 97%, 98%, or 99% identical to the N1 NA polypeptide of SEQ ID NO: 1, wherein the non-naturally occurring polypeptide includes 1, 2, 3, 4, 5, 6, or all 7 of the following amino acid residues relative to SEQ ID NO:1 when aligned by protocol 1 or protocol 2:
161T/A, 105A, 165T/I, 166P, 196Q, 203Y, 444V; or

(b) at least 75%, 80%, 85%, 90%, 91%, 92%, 93%, 94%, 95%, 96%, 97%, 98%, or 99% identical to the N1 NA polypeptide of SEQ ID NO: 1, wherein the non-naturally occurring polypeptide includes 2, 3, 4, 5, 6, 7, 8, 9, 10, 11, 12, 13, 14, 15, 16, or all 17 of the following amino acid residues relative to SEQ ID NO:1 when aligned by protocol 1 or protocol 2:
161T/A/V/S/T, 100L, 408M, 419V, 99P, 103N, 105A, 131Q/M, 163I/L, 165T/S/V/A/I, 166P, 177I, 196Q/T, 203Y, 205I, 442I, 444V.

In this first aspect, the N1 NA reference sequence is based on N1 reference strain A/California/07/2009 from H1N1.

(SEQ ID NO: 1)
MNPNQKIITIGSVCMTIGMANLILQIGNIISIWISHSIQLGNQNQIETCN
QSVITYENNTWVNQTYVNISNTNFAAGQSVVSVKLAGNSSLCPVSGWAIY
SKDNSVRIGSKGDVFVIREPFISCSPLECRTFFLTQGALLNDKHSNGTIK
DRSPYRTLMSCPIGEVPSPYNSRFESVAWSASACHDGINWLTIGISGPDN
GAVAVLKYNGIITDTIKSWRNNILRTQESECACVNGSCFTVMTDGPSNGQ
ASYKIFRIEKGKIVKSVEMNAPNYHYEECSCYPDSSEITCVCRDNWHGSN
RPWVSFNQNLEYQIGYICSGIFGDNPRPNDKTGSCGPVSSNGANGVKGFS
FKYGNGVWIGRTKSISSRNGFEMIWDPNGWTGTDNNFSIKQDIVGINEWS
GYSGSFVQHPELTGLDCIRPCFWVELIRGRPKENTTWTSGSSISFCGVNS
DTVGWSWPDGAELPFTIDK,
wherein the bold region is the head region of the
N1 HA protein

As used throughout this application (for all NA subtypes). “Protocol 1” and “Protocol 2” both permit alignment of polypeptide against the reference sequence (SEQ ID NO:1 in the above embodiment; SEQ ID NOs:2-9 in embodiments described below), taking insertions and deletions into account. Thus, the percent identity requirement is based on alignment with the reference sequence while discounting insertions or deletions relative to the reference polypeptide.

### Protocol 1

1. To run a BLASTp alignment online, use the National Center for Biotechnology Information (NCBI) server (or see the article https://www.ncbi.nlm.nih.gov/pmc/articles/PMC146917/).
   a. https://blast.ncbi.nlm.nih.gov/Blast.cgi?PAGE=Proteins
2. Set up a BLAST alignment using the following settings:
   Use the option for “Align two or more sequences”
   Enter the subtype-specific reference strain sequence for the relevant NA subtype into the “Enter Query Sequence” section
   Enter any sequence of the same NA subtype into the “Enter Subject Sequence” section
   Algorithm: blastp (protein-protein BLAST)
   Expect threshold: 0.1
   Word size: 6
   Max matches in a query range: 0
   Matrix; BLOSUM62
   Gap costs:
      Existence: 11
      Extension: 1
   Filter low complexity regions?: No
   Mask:
      For lookup table only?: No
      Lower case letters?: No
3. Run the analysis by clicking the “BLAST” button
4. Click on the “Alignments” tab to show the alignment between the two sequences
5. For each sequence position of interest, identify the number according to the “Query” sequence. Then identify the corresponding residue position in the “Sbjet” sequence that has been aligned with the position of the “Query” sequence.

### Protocol 2

1. To run a protein BLASTp alignment on a personal computer or server, install BLAST for command line execution or identify a computer or server that already has it installed.
   a. Directions for installation can be found in the user manual: “BLAST® Command Line Applications User Manual”, National Center for Biotechnology Information (US). Bethesda, Md. 2008. https://www.ncbi.nlm.nih.gov/books/NBK279690/
2. Generate a file in FASTA format that contains the desired subtype-specific reference strain for the relevant NA subtype. In the command below, this file will be named “query.fosta”.
3. Generate a second file in FASTA format that contains an N A sequence of interest from the same subtype. In the command below, this file will be named “sbjct.fasta”.
4. Execute the following command using a program such as Terminal, iTerm2, Windows Console, Linux console or other similar terminal emulators. This will generate results in a file named “results.txt”.
   blastp-query query.fasta-subject sbjct.fasta-matrix BLOSUM62-evalue 0.1-word_size 6-gapopen 11-gapextend 1-out results.txt
5. Open results.txt and view the section showing alignment of the two sequences. For each sequence position of interest, identify the number according to the “Query” sequence. Then identify the corresponding residue position in the “Sbjet” sequence that has been aligned with the position of the “Query” sequence.

In another embodiment of any aspect, embodiment or combination of embodiments described herein (for all NA subtypes), the percent identity is based on an alignment of the polypeptide to the reference sequence using any protocol, and insertions and deletions relative to the reference polypeptide are not considered in determining percent identity. In a further embodiment or combination of embodiments described herein, the percent identity is based on an alignment of the polypeptide to the reference sequence using any protocol, and insertions and deletions relative to the reference polypeptide are considered in determining percent identity.

Throughout the application (for all NA subtypes), mutations that primarily improve expression of the engineered N A polypeptides are denoted in bold font.

Throughout the application (for all NA subtypes), mutations that primarily destabilize the engineered NA tetramers are denoted in italicized font.

In one embodiment of this first aspect, the polypeptides are

(a) at least 75%, 80%, 85%, 90%, 91%, 92%, 93%, 94%, 95%, 9*%, 97%, 98%, or 99% identical to the N1 NA polypeptide of SEQ ID NO: 1, and wherein the non-naturally occurring polypeptide includes 1, 2, 3, 4, 5, 6, or all 7 of the following amino acid residues relative to SEQ ID NO:1 when aligned by protocol 1 or protocol 2:

(i) 161T/A, 105A, 157K, 165T/I, 166P, 196Q, 203Y, 444V; or

(ii) 105A, 165T/I, 166P, 196Q, 203Y, 444V.

In another embodiment of the first aspect, the polypeptides are

(b) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 2, 3, 4, 5, 6, 7, 8, 9, 10, 11, 12, 13, 14, 15, 16, or all 17 of the following amino acid residues relative to SEQ ID NO:1 when aligned by protocol 1 or protocol 2:

(i) 161T/A/V/S/T, 100L, 408M, 419V, 99P, 103N, 105A, 131Q/M, 163I/L, 165T/S/V/A/I, 166P, 177I, 196Q/T, 203Y, 205I, 442I, 444V; or

(ii) 99P, 103N, 105A, 131Q/M, 163I/L, 165T/S/V/A/I, 166P, 177I, 196Q/T, 203Y, 205I, 442I, 444V.

In a further embodiment of this first aspect, the polypeptides include one or more of the following sets of amino acid residues relative to SEQ ID NO: 1 when aligned by protocol 1 or protocol 2:

(i)
161V/172A;
100L/408M;
100L/408M/419V
103N/105A;
114L/166P;
101C/164C;
101C/164C/174V;
101C/122V/164C/174V/444V;
122V/444V;
131Q/442I;
101A/163L/444A;
131M/442I;
101A/131M/163L/442I/444A;
100L/101A/131M/163L/442I/444A
131M/163L/442I/444A;
100L/131M/163L/442I/444A;
99P/100L/161V/165S/172A/177I/196T/205I/408M/419V;
99P/100L/161V/165S/172A/177I/196T/408M/419V;
99P/100L/131Q/161V/165S/172A/177I/196T/205I/408M/419V;
99P/100L/131Q/161V/165S/172A/177I/196T/408M/419V;
99P/100L/101A/131M/161V/163L/165S/172A/177I/196T/205I/408M/419V 442I/444A;
99P/100L/101A/131M/161V/163L/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T;
99P/101A/131M/161V/163L/165S/172A/177I/196T/205I/442I/444A;
99P/101A/131M/161V/163L/165S/172A/177I/196T/442I/444A;
99P/196T;
99P/196T/205I;
99P/177I/196T; and/or
99P/177I/196T/205I; or
   (ii)
103N/105A;
114I/166P;
101C/164C;
101C/164C/174V;
101C/122V/164C/174V/444V;
122V/444V;
131Q/442I;
101A/163L/444A;
131M/442I;
101A/131M/163L/442I/444A;
100L/101A/131M/163L/442I/444A;
131M/163L/442I/444A;
100L/131M/163L/442I/444A;
99P/100L/161V/165S/172A/177I/196T/205I/408M/419V;
99P/100L/131Q/161V/165S/172A/177I/196T/205I/408M/419V;
99P/100L/131Q/161V/165S/172A/177I/196T/419V;
99P/100L/101A/131M/161V/163L/165S/172A/177I/196T/205I/408M/419V/442I/444A;
99P/100L/101A/131M/161V/163L/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T;
99P/101A/131M/161V/163L/165S/172A/177I/196T/205I/442I/444A;
99P/101A/131M/161V/163L/165S/172A/177I/196T/442I/444A;
99P/196T;
99P/196T/205I;
99P/177I/196T; and or
99P/177I/196T/205I.

In various embodiments, of this first aspect, the polypeptides includes 3, 4, 5, 6, 7, 8, 9 or more of the listed amino acid residues relative to SEQ ID NO: 1 when aligned by protocol 1 or protocol 2.

In a further embodiment, the polypeptides further comprises 1, 2, 3, 4, 5, or all 6 of the following residues relative to SEQ ID NO:1 when aligned by protocol 1 or protocol 2:
105S, 106V, 163I, 166V, 210G, 442S.

In a still further embodiment of the first aspect, the polypeptides include one or more of the following sets of amino acid residues relative to SEQ ID NO:1 when aligned by protocol 1 or protocol 2:
99P/100L/161V/165S/172A/177I/196T/205I/408M/419V;
99P/100L/161V/165S/172A/177I/196T/408M/419V;
99P/100L/131Q/161V/165S/172A/177I/196T/205I/408M/419V;
99P/100L/131Q/161V/165S/172A/177I/196T/408M/419V;
99P/100L/101A/131M/161V/163L/165S/172A/177I/196T/205I/408M/419V/442I/444A;
99P/100L/101A/131M/161V/163L/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T/;
99P/101A/131M/161V/163L/165S/172A/177I/196T/205I/442I/444A; and/or
99P/101A/131M/161V/163L/165S/172A/177I/196T/442I/444A.

In a still further embodiment, the polypeptides comprises an amino acid sequence at least 75%, 80%, 85%, 90%, 91%, 92%, 93%, 94%, 95%, 96%, 97%, 98%, or 99% identical to the amino acid selected from the group consisting of NI mutants listed in Table 1 (SEQ ID NO: 10-32 and 39-95), when aligned by protocol 1 or protocol 2, wherein the polypeptide includes all of the residues listed in Table 1 for an individual NI mutant listed in Table 1

In a second aspect, the NA polypeptides are:

(a) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 1, 2, 3, 4, 5, 6, 7, or all 8 of the following amino acid residues relative to SEQ ID NO:2 when aligned by protocol 1 or protocol 2:
101C, 131M, 162P, 165S/T, 166V, 195Q, 202Y, 443S; or

(bl at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 2, 3, 4, 5, 6, 7, 8, 9, 10, 11, or all 12 of the following amino acid residues relative to SEQ ID NO:2 when aligned by protocol 1 or protocol 2:
101C/A, 103N, 105A, 106V, 131M, 162P, 163I, 165S/T/A/I, 166V, 195Q, 202Y, 443S.

In this second aspect, the N2 NA reference sequence is based on N2 reference strain A/Wisconsin/67/2005 from H3N2.

(SEQ ID NO: 2)
MNPNQKIITIGSVSLTISTICFFMQIAILITTVTLHFKQYEFNSPPNNQV
MLCEPTIIERNITEIVYLTNTTIEKEICPKLAEYRNWSKPQCNITGFAPF
SKDNSIRLSAGGDIWVTREPYVSCDPDKCYQFALGQGTTLNNVHSNDTVH
DRTPYRTLLMNELGVPFHLGTKQVCIAWSSSSCHDGKAWLHVCVTGDDKN
ATASFIYNGRLVDSIVSWSKEILRTQESECVCINGTCTVVMTDGSASGKA
DTKILFIEEGKIVHTSTLSGSAQHVEECSCYPRYLGVRCVCRDNWKGSNR
PIVDINIKDYSIVSSYVCSGLVGDTPRKNDSSSSSHCLDPNNEEGGHGVK
GWAFDDGNDVWMGRTISEKLRSGYETFKVIEGWSNPNSKLQINRQVIVDR
GNRSGYSGIFSVEGKSCINRCFYVELIRGRKEETEVLWTSNSIVVFCGTS
GTYGTGSWPDGADINLMPI,
wherein the highlighted residues are the head
region

In one embodiment of this second aspect, the polypeptides are:

(a) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 1, 2, 3, 4, 5, 6, 7, or all 8 of the following amino acid residues relative to SEQ ID NO:2 when aligned by protocol 1 or protocol 2:
101C, 131M, 162P, 165S/T, 166V, 195Q, 202Y, 443S.

In another embodiment, the polypeptides are:

(b) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 2, 3, 4, 5, 6, 7, 8, 9, 10, 11, or all 12 of the following ammo acid residues relative to SEQ ID NO:2 when aligned by protocol 1 or protocol 2:
101C/A, 103N, 105A, 106V, 131M, 162P, 163I, 165S/T/A/I, 166V, 195Q, 202Y, 443S.

In another embodiment of this second aspect, the polypeptides include one or more of the following sets of amino acid residues relative to SEQ ID NO:2 when aligned by protocol 1 or protocol 2:
103N/105A;
101C/164C;
101C/164C/173V;
101A/131M;
100L/101A/131M; and/or
100L/131M/163L.

In various embodiments of this second aspect, the polypeptides include 3, 4, 5, 6, 7, 8, 9, or more of the listed amino acid residues relative to SEQ ID NO:2 when aligned by protocol 1 or protocol 2. In another embodiment, the polypeptides of this second aspect comprises the amino acid sequence at least 75%, 80%, 85%, 90%, 91%, 92%, 93%, 94%, 95%, 96%, 97%, 98%, or 99% identical to the amino acid selected from the group consisting of N2 mutants listed in Table 1 (SEQ ID NO: 33-34), when aligned by protocol 1 or protocol 2, wherein the polypeptide includes all of the residues listed in Table 1 for an individual N2 mutant listed in Table 1. In a still further embodiment of this second aspect, the polypeptides are at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes a 165Q/E amino acid residues relative to SEQ ID NO:2 when aligned by protocol 1 or protocol 2, or includes 1 or 3 of 165Q/E, 176V, 195S amino acid residues relative to SEQ ID NO:2 when aligned by protocol 1 or protocol 2.

In a third aspect, the NA polypeptides are:

(a) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 1, 2, 3, 4, 5, 6, 7, 8, 9, 10, or all 11 of the following amino acid residues relative to SEQ ID NO:3 when aligned by protocol 1 or protocol 2:
103N, 105S, 131Q/M, 157T, 165S/I, 166P, 196Q, 203Y, 205I, 443S, 445V; or

(b) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 2, 3, 4, 5, 6, 7, 8, 9, 10, 11, 12, or all 13 of the following amino acid residues relative to SEQ ID NO:1 when aligned by protocol 1 or protocol 2:
103N, 105S, 106V, 131Q/M, 157T, 163L, 165S/I/V/A, 166P/V, 196Q, 203Y, 205T, 443S, 445V.

In this third aspect, the N3 NA reference sequence is based on N3 reference strain A/Swine/Missouri/2124514/2006 from H2N3.

(SEQ ID NO: 3)
MNPNQRIITIGVVNTTLSTIALLIGVGNLVFNTVIHEKIGDHQIVTYPII
TTPAVPNCSDTIITYNNTVINNITTTIITEEERPFKSPLPLCPFRGFFPF
HKDNAIRLGENKDVIVTREPYVSCDNDNCWSFALAQGALLGTKHSNGTIK
DRTPYRSLIRFPIGTAPVLGNYKEICIAWSSSSCFDGKEWMHVCMTGNDN
DASAQIIYGGRMTDSIKSWRKDILRTQESECQCIDGTCVVAVTDGPAANS
ADYRVYWIREGKIIKYENVPKTKIQHLEECSCYVDIDVYCICRDNWKGSN
RPWMRINNETILETGYVCSKFHSDTPRPADPSTMSCDSPSNVNGGPGVKG
FGFKAGDDVWLGRTVSTSGRSGFEIIKVTEGWINSPNHVKSITQTLSNND
WSGYSGSFIVKAKDCFQPCFYVELIRGRPNKNDDVSWTSNSIVTFCGLDN
EPGSGNWPDGSNIGFMPK
(Head region indicated by highlighting)

In one embodiment of this third aspect, the polypeptides are:

(a) at least 75%, 80%, 85%, 90%, 91%, 92%, 93%, 94%, 95%, 96%, 97%, 98%, or 99% identical to the N3 NA polypeptide of SEQ II) NO:3, and wherein the non-naturally occurring polypeptide includes 1, 2, 3, 4, 5, 6, 7, 8, 9, 10, or all 11 of the following amino acid residues relative to SEQ ID NO:3 when aligned by protocol 1 or protocol 2:
103N, 105S, 131Q/M, 157T, 165S/I, 166P, 196Q, 203Y, 205I, 443S, 445V.

In another embodiment, the polypeptides are

(b) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 2, 3, 4, 5, 6, 7, 8, 9, 10, 11, 12, or all 13 of the following amino acid residues relative to SEQ ID NO:3 when aligned by protocol 1 or protocol 2:
103N, 105S, 106V, 131Q/M, 157T, 163L, 165S/I/V/A, 166P/V, 196Q, 203Y, 205I, 443S, 445V.

In another embodiment of this third aspect, the polypeptide includes one or more of the following sets of amino acid residues relative to SEQ ID NO:3 when aligned by protocol 1 or protocol 2:
101C/164C;
101C/164C/174V;
101C/164C/174V/445V;
101A/445V;
101A/131M/445A;
131M/445A;
114I/166P;
101A/163L/445V;
101A/131M/163L/445A; and/or
131M/163L/445A.

In various further embodiments of this third aspect, the polypeptides includes 3, 4, 5, 6, 7, 8, 9 or more of the listed amino acid residues relative to SEQ ID NO:3 when aligned by protocol 1 or protocol 2. In another embodiment, the polypeptide is at least 75%, 80%, 85%, 90%, 91%, 92%, 93%, 94%, 95%, 96%, 97%, 98%, or 99% identical to the N3 NA polypeptide of SEQ ID NO: 3, and wherein the non-naturally occurring polypeptide includes one or both of the following amino acid residues relative to SEQ ID NO: 3 when aligned by protocol 1 or protocol 2: 196S, 165Q/E.

In a fourth aspect, the NA polypeptides are:

(a) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 1, 2, 3, 4, 5, 6, 7, 8, 9, 10, 11, 12, 13, 14, 15, or all 16 of the following amino acid residues relative to SEQ ID NO:4 when aligned by protocol 1 or protocol 2:
160V/S/T/A, 409M, 102N, 104S/A, 105V, 112D, 130Q/M, 162L, 164S/T/A/I, 165P, 176I, 195Q, 202Y, 204I, 443I, 445V; or

(b) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 2, 3, 4, 5, 6, 7, 8, 9, 10, 11, 12, 13, 14, 15, or all 16 of the following amino acid residues relative to SEQ ID NO:4 when aligned by protocol 1 or protocol 2:
160V/S/T/A, 409M, 102N, 104S/A, 105V, 112D, 130Q/M, 162L, 164ST/A/I, 165PV, 176I, 195Q, 202Y, 204I, 443I, 445V

In this fourth aspect, the N4 NA reference sequence is based on N4 reference strain A/ruddy turnstone/Delaware Bay/141/2016 from H10N4.

(SEQ ID NO: 4)
MNPNQKIITIGSVSIILTTVGLLLQITSLCSIWFSHYNQVAQTHEQPCSN
NTTNYYNETFVNVTNVQNNYTTIAEPSAPDVVHYSSGRDLCPIRGWAPLS
KDNGIRIGSRGEVFVIREPFISCSISECRTFFLTQGALLNDKHSNGTVKD
RSPFRTLMSCPIGVAPSPSNSRFESVAWSATACSDGPGWLTLGITGPDST
AVAVLKYNGIITDTLKSWKGNIMRTQESECVCQDEFCYTLVTDGPSDAQA
FYKILKIRKGKIVSMKDVDATGFHFEECSCYPSGTEIECVCRDNWRGSNR
PWIRFNSDLDYQIGYVCSGIFGDNPRPVDGTGSCNGPVNNGKGRYGVKGF
SFRYGDGVWIGRTKSLESRSGFEMVWDANGWVSTDKDSNGVQDIIDNDNW
SGYSGSFSIRGETTGKNCTVPCFWVEMIRGQPKEKTIWTSGSSIAFCGVN
SDTTGWSWPDGALLPFDIDK
(Head sequence highlighted)

In one embodiment of this further aspect, the polypeptides are

(a) at least 75%, 80%, 85%, 90%, 91%, 92%, 93%, 94%, 95%, 96%, 97%, 98%, or 99% identical to the N4 NA polypeptide of SEQ ID NO:4, and wherein the lion-naturally occurring polypeptide includes 1, 2, 3, 4, 5, 6, 7, 8, 9, 10, 11, 12, 13, 14, 15, or all 16 of the following amino acid residues relative to SEQ ID NO:4 when aligned by protocol 1 or protocol 2:

(i) 160V/S/T/A, 409M, 102N, 104S/A, 105V, 112D, 130Q/M, 162L, 164S/T/A/I, 165P, 176I, 195Q, 202Y, 204I, 443I, 445V; or

(ii) 102N, 104S/A, 105V, 112D, 130Q/M, 162L, 164S/T/A/I, 165P, 176I, 195Q, 202Y, 204I, 443I, 445V.

In another embodiment, the polypeptides are

(b) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 2, 3, 4, 5, 6, 7, 8, 9, 10, 11, 12, 13, 14, 15, or all 16 of the following amino acid residues relative to SEQ ID NO:4 when aligned by protocol 1 or protocol 2:

(i) 160V/S/T/A, 409M, 102N, 104S/A, 105V, 112D, 130Q/M, 162L, 164S/T/A/I, 165P/V, 176I, 195Q, 202Y, 204I, 443I, 445V; or

(ii) 102N, 104S/A, 105V, 112D, 130Q/M, 162L, 164S/T/A/I, 165P/V, 176I, 195Q, 202Y, 204I, 443I, 445V.

In another embodiment of this fourth aspect, the polypeptides include one or more of the following sets of amino acid residues relative to SEQ ID NO:4 when aligned by protocol 1 or protocol 2:

(i)
160V/S/T/A;
160V/171A;
102N/104A;
100C/163C;
100C/163C/173V;
100C/163C/173V/445V;
130Q/443I;
100A/162L/445A;
130M/443I;
100A/130M/162L/443I/445A;
130M/162L/443I/445A;
176I/204I;
100C/121V/163C/173V/445V; and/or
121V/445V; or

(ii)
102N/104A;
100C/163C;
100C/163C/173V;
100C/163C/173V/445V;
130Q/443I;
100A/162L/445V;
130M/443I;
100A/130M/162L/443I/445A;
130M/162L/443I/445A;
176I/204I;
100C/121V/163C/173V/445V; and/or
121V/445V.

In various further embodiments of this fourth aspect, the polypeptides include 3, 4, 5, 6, 7, 8, 9 or more of the listed amino acid residues relative to SEQ ID NO:4 when aligned by protocol 1 or protocol 2. In a further embodiment, the polypeptides are at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 1, or both of the following amino acid residues relative to SEQ ID NO:4 when aligned by protocol 1 or protocol 2: 164Q/E, 195S.

In a fifth aspect, the NA polypeptides are:

(a) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, 2, 3, 4, 5, 6, 7, 8, 9, 10, 11, 12, or all 13 of the following amino acid residues relative to SEQ ID NO:5 when aligned by protocol 1 or protocol 2:
97L, 410M, 96P, 98A, 100N, 102S/A, 110D, 128Q/M, 160I, 162V/A/I, 163V/P, 193Q/T, 445I; or

(b) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 2, 3, 4, 5, 6, 7, 8, 9, 10, 11, 12, 13, 14, 15, or all 16 of the following amino acid residues relative to SEQ ID NO:5 when aligned by protocol 1 or protocol 2:

97L, 410M, 96P, 98A, 100N, 102S/A, 103V, 110D, 128Q/M, 154T, 160I, 162V/A/I/T, 163V/P, 174I, 193Q/T, 445I.

In this fifth aspect, the N5 NA reference sequence is based on N5 reference strain A/gull/Delaware Bay/218/2016 from H10N5.

(SEQ ID NO: 5)
MNPNQKIITIGSVSLALVVFNILLHIASIVIGIISVTKEISVSSTCNTTE
VYVETVRLETITIPINNTVYIERESHQEPEFLNNTEPLCNVSGFAIVSKD
NGIRIGSRGHVFVIREPFVACGPTECRTFFLTQGALLNDKHSNNTVKDRS
PYRALMSVPLGSSPNAYQAKFESVAWSATACHDGKRWLAVGISGADDDAY
AVIHYGGMPTDVVRSWRKQILRTQESSCVCMKGNCYWVMTDGPANSQASY
KIFKSHKGMVTNEREVSFQGGHIEECSCYPNLGKVECVCRDNWNGMNRPV
LTFDEDLNYEVGYLCAGIPTDTPRVQDNSFIGSCTNAVGGSGTNNYGVKG
FGFRQGNSVWAGRTVSISSRSGFEILLVEDGWVKTSKNVVKKVEVLNNKN
WSGYSGAFTIPITMTSKQCLVPCFWLEMIRGKPEERTSIWTSSSSTVFCG
VSSEVPGWSWDDGAILPFDIDKM
(Head sequence is highlighted)

In one embodiment of this fifth aspect, the polypeptides are

(a) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, 2, 3, 4, 5, 6, 7, 8, 9, 10, 11, 12, or all 13 of the following amino acid residues relative to SEQ ID NO:5 when aligned by protocol 1 or protocol 2:
(i) 97L, 410M, 96P, 98A, 100N, 102S/A, 110D, 128Q/M, 160I, 162V/A/I, 163V/P, 193Q/T, 445I; or

(ii) 96P, 98A, 100N, 102S/A, 110D, 128Q/M, 160I, 162V/A/I, 163V/P, 193Q/T, 445I.

In another embodiment of this fifth aspect, the polypeptides are

(b) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 2, 3, 4, 5, 6, 7, 8, 9, 10, 11, 12, 13, 14, 15, or all 16 of the following amino acid residues relative to SEQ ID NO:5 when aligned by protocol 1 or protocol 2:

(i) 97L, 410M, 96P, 98A, 100N, 102S/A, 103V, 110D, 128Q/M, 154T, 160I, 162V/A/I/T, 163V/P, 174I, 193Q/T, 445I; or

(ii) 96P, 98A, 100N, 102S/A, 103V, 110D, 128Q/M, 154T, 160I, 162V/A/I/T, 163V/P, 174I, 193Q/T, 445I.

In other embodiments of this fifth aspect, the polypeptides include one or more of the following sets of amino acid residues relative to SEQ ID NO:5 when aligned by protocol 1 or protocol 2:

(i)
97L/410M;
100N/102A;
98C/161C;
128Q/445I;
128M/445I;
98A/128M/445I/447A;
97L/98A/128M/445I/447A;
128M/445I/447A;
97L/128M/445I/447A;
96P/193T;
111I/163P;
96P/193T/202I;
96P/174I/193T; or
96P/174I/193T/202I; or

(ii)
100N/102A;
98C/161C;
128Q/445I;
128M/445I;
98A/128M/445I/447A;
97L/98A/128M/445I/447A;
128M/445I/447A;
97L/128M/445I/447A;
96P/193T;
111I/163P;
96P/193T/202I;
96P/174I/193T; and/or
96P/174I/193T/202I.

In further embodiments of this fifth aspect, the polypeptides includes 3, 4, 5, 6, 7, 8, 9 or more of the listed amino acid residues relative to SEQ ID NO:5 when aligned by protocol 1 or protocol 2. In a further embodiment, the polypeptides are at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, or both of the following amino acid residues relative to SEQ ID NO:5 when aligned by protocol 1 or protocol 2: 162 Q/E, 200S.

In a sixth aspect, the NA polypeptides are:

(a) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 1, 2, 3, 4, 5, 6, 7, 8, 9, 10, or all 11 of the following amino acid residues relative to SEQ ID NO:6 when aligned by protocol 1 or protocol 2:
99P, 103N, 113D, 131Q, 161I, 162P, 163I, 165S/T/V/A, 196Q, 203V, 445S; or

(b) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 2, 3, 4, 5, 6, 7, 8, 9, 10, 11, 12, 13, 14, 15, or all 16 of the following amino acid residues relative to SEQ ID NO:6 when aligned by protocol 1 or protocol 2:
99P, 103N, 105S, 106V, 113D, 131Q, 157T, 161I, 162P, 163I/L, 165S/T/V/A/I, 166V/P, 196Q, 203Y, 445S.

In this sixth aspect, the N6 NA reference sequence is based on N6 reference strain A/chicken/Sichuan/NCJPLI/2014 from H5N6.

(SEQ ID NO: 6)
MNPNQKITCISATSMTLSVVSMLIGIANLGLNIGLHYKVSDSTNINIPNM
NETSPTTTIINNHPQNNFTNTTNIIVTKTEEGHLLNLTKPLCEVNSWNIL
SKDNAIRIGEDAHIIVTREPYLSCDPQGCRMFALSQGTTLRGKHANGTIH
DRSPFRALVSWEMGQAPSPYNTRVECIGWSSTSCHDGISRMSICISGPNN
NASAVVWYGGRPVTEIPSWAGNILRTQESECVCHGGICPVVMTDGPANNR
AETKIIYFKEGKIKKIEELKGDAQHIEECSCYGASEMIKCICRDNWKGAN
RPVITIDPEMMTHTSKYLCSKILTDTSRPNDPTNGKCEAPITGGSPDPGV
KGFAFLDGENSWLGRTISKDSRSGYEMLKVPNAETDTQSGAISHQIIVNN
QNWSGYSGAFIDYWANKECFNPCFYVELIRGRPKESSVLWTSNSIVALCG
SKERLGSWSWHDGAEIIYFK
(Head sequence highlighted)

In one embodiment of this sixth aspect, the polypeptides are:

(a) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 1, 2, 3, 4, 5, 6, 7, 8, 9, 10, or all 11 of the following amino acid residues relative to SEQ ID NO:6 when aligned by protocol 1 or protocol 2:
99P, 103N, 113D, 131Q, 161I, 162P, 163I, 165S/T/V/A, 196Q, 203Y, 445S.

In another embodiment of this sixth aspect, the polypeptides are:

(b) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 2, 3, 4, 5, 6, 7, 8, 9, 10, 11, 12, 13, 14, or all 15 of the following amino acid residues relative to SEQ ID NO:6 when aligned by protocol 1 or protocol 2:

99P, 103N, 105S, 106V, 113D, 131Q, 157T, 161I, 162P, 163I/L, 165S/T/V/A/I, 166V/P, 196Q, 203Y, 445S.

In various further embodiments of this sixth aspect, the polypeptide include one or more of the following sets of ammo acid residues relative to SEQ ID NO:6 when aligned by protocol 1 or protocol 2:
101C/164C;
101C/164C/174V;
101 C/164C/174 V/447V;
122V/447V;
101A/163L;
99P/196T; and or
99P/196T/205I.

In further embodiments, the polypeptides include 3, 4, 5, 6,7, 8, 9 or more of the listed amino acid residues relative to SEQ ID NO:6 when aligned by protocol 1 or protocol 2. In another embodiment, the polypeptides areal least 75%, 80%, 85%, 90%, 91%, 92%, 93%, 94%, 95%, 96%, 97%, 98%, or 99% identical to the N6 NA polypeptide of SEQ ID NO:6, wherein the non-naturally occurring polypeptide includes a 165E amino acid mutation relative to SEQ ID NO:6 when aligned by protocol 1, and optionally also includes a 177V amino acid mutation relative to SEQ ID NO:6 when aligned by protocol 1 or protocol 2.

In a seventh aspect, the NA polypeptides are:

(a) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 1, 2, 3, 4, 5, 6, 7, 8, 9, 10, or all 11 of the following amino acid residues relative to SEQ ID NO:7 when aligned by protocol 1 or protocol 2:
98P, 102N, 104S, 112D, 130Q, 156T, 162I, 164V/A/I, 165V, 202Y, 448V; or

(b) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 2, 3, 4, 5, 6, 7, 8, 9, 10, 11, 12, or all 13 of the following amino acid residues relative to SEQ ID NO:7 when aligned by protocol 1 or protocol 2:
98P, 102N, 104S, 112D, 130Q, 156T, 162I, 164V/A/I, 165V, 176I, 202Y, 446I, 448V.

In this seventh aspect, the N7 NA reference sequence is based on N7 reference strain A/Netherlands/219/2003 from H7N7.

(SEQ ID NO: 7)
MNPNQRLFALSGVAIALSVLNLLIGISNVGLNVSLHLKEKGPKQEENLTC
TTINQNNTTVVENTYVNNTTIITKGTDLKTPSYLLLNKSLCNVEGWVVIA
KDNAVRFGESEQIIVTREPYVSCDPTGCKMYALHQGTTIRNKHSNGTIHD
RTAFRGLISTPLGTPPTVSNSDFMCVGWSSTTCHDGIARMTICIQGNNDN
ATATVYYNRRLTTTIKTWARNILRTQESECVCHNGTCAVVMTDGSASSQA
YTKVMYFHKGLVVKEEELRGSARHIEECSCYGHNQKVTCVCRDNWQGANR
PIIEIDMSTLEHTSRYVCTGILTDTSRPGDKSSGDCSNPITGSPGVPGVK
GFGFLNGDNTWLGRTISPRSRSGFEMLKIPNAGTDPNSRIAERQEIVDNN
NWSGYSGSFIDYWNDNSECYNPCFYVELIRGRPEEAKYVWWASNSLIALC
GSPFPVGSGSFPDGAQIQYFS
(Head sequence highlighted)

In one embodiment of this seventh aspect, the polypeptides are

(a) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 1, 2, 3, 4, 5, 6, 7, 8, 9, 10, or all 11 of the following amino acid residues relative to SEQ ID NO:7 when aligned by protocol 1 or protocol 2:
98P, 102N, 104S, 112D, 130Q, 156T, 162I, 164V/A/I, 165V, 202Y, 448V.

In another embodiment, the polypeptides are

(b) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 2, 3, 4, 5, 6, 7, 8, 9, 10, 11, 12, or all 13 of the following amino acid residues relative to SEQ ID NO:7 when aligned by protocol 1 or protocol 2:
98P, 102N, 104S, 112D, 130Q, 156T, 162I, 164V/A/I, 165V, 176I, 202Y, 446I, 448V.

In various farther embodiments of this seventh aspect, the polypeptide includes one or more of the following sets of amino acid residues relative to SEQ ID NO: 7 when aligned by protocol 1 or protocol 2:
100C/163C;
100C/163C/173V;
100C/163C/173V/448V;
99L/446I/448A;
98P/195T;
130Q/446I;
446I/44KA;
98P/195T/204I;
98P/176I/195T; and/or
98P/176I/195T/204I.

In further embodiments, the polypeptides include 3, 4, 5, 6, 7, 8, 9, or more of the listed amino acid residues relative to SEQ ID NO: 7 when aligned by protocol 1 or protocol 2. In another embodiment, the polypeptides are at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes one or both of the following amino acid mutation relative to SEQ ID NO:7 when aligned by protocol 1 or protocol 2:
164Q/E, 195S.

In an eighth aspect, the NA polypeptides are:

(a) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 1, 2, 3, 4, 5, 6, 7, 8, 9, 10, or all 11 of the following amino acid residues relative to SEQ ID NO:8 when aligned by protocol 1 or protocol 2:
408M, 101N, 103A/S, 111D, 129Q, 161P/L, 163S/T/V/A/I, 164V, 194Q, 201Y, 442I; or

b) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 2, 3, 4, 5, 6, 7, 8, 9, 10, 11, 12, 13, 14, or all 15 of the following amino acid residues relative to SEQ ID NO:8 when aligned by protocol 1 or protocol 2:
98L, 408M, 101N, 103A/S, 104V, 111D, 129Q/M, 161P/L, 163S/T/V/A/I/S/T, 164V/P, 175I, 194Q, 201Y, 203I, 442I.

In this eighth aspect, the N8 NA reference sequence is based on N8 reference strain: A/Jiangxi/IPB13b/2013 from H10N8.

(SEQ ID NO: 8)
MNPNQKIITIGSVSLGLVILNILLHIVSITVTVLVLPGNGNNESCNETVI
REYNETVRVEKVTQWHNTNVIEYIERPENDHFMNNTEALCDAKGFAPFSK
DNGIRIGSRGHVFVIREPFVSCSPTECRTFFLTQGSLLNDKHSNGTVKDR
SPYRTLMSVEIGQSPNVYQARFEAVAWSATACHDGKKWMTIGVTGPDAKA
VAVVHYGGIPTDVINSWAGDILRTQESSCTCIQGECFWVMTDGPANRQAQ
YRAFKAKQGKIVGQAEISFNGGHIEECSCYPNEGKVECVCKDNWTGTNRP
VLVISPDLSYRVGYLCAGLPSDTPRGEDSQFTGSCTSPMGNQGYGVKGFG
FRQGNDVWMGRTISRTSRSGFEILKVRNGWVQNSKEQIKRQVVVDNLNWS
GYSGSFTLPAELTKRNCLVPCFWVEMIRGNPEEKTIWTSSSSIVMCGVDH
EIADWSWHDGAILPFDIDKM
(Head sequence highlighted)

In one embodiment of this eighth aspect, the polypeptides are:

(a) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 1, 2, 3, 4, 5, 6, 7, 8, 9, 10, or all 11 of the following amino acid residues relative to SEQ ID NO:8 when aligned by protocol 1 or protocol 2:

(i) 408M, 101N, 103A/S, 111D, 129Q, 160P, 161L, 163S/T/V/A/I, 164V, 194Q, 201Y, 442I; or

(ii) 101N, 103A/S, 111D, 129Q, 160P, 161L, 163S/T/V/A/I, 164V, 194Q, 201Y, 442I.

In another embodiment, the polypeptides are

(b) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 2, 3, 4, 5, 6, 7, 8, 9, 10, 11, 12, 13, 14, or all 15 of the following amino acid residues relative to SEQ ID NO:8 when aligned by protocol 1 or protocol 2:

(i) 98L, 408M, 101N, 103A/S, 194V, 111D, 129Q/M, 160P, 161L, 163S/T/V/A/I/S/T, 164V/P, 175I, 194Q, 201Y, 203I, 442I; or

(ii) 101N, 103A/S, 104V, 111D, 129Q/M, 160P, 161L, 163S/T/V/A/I/S/T, 164V/P, 175I, 194Q, 201Y, 203I, 442I

In various further embodiments, the polypeptide include one or more of the following sets of amino acid residues relative to SEQ ID NO: 8 when aligned by protocol 1 or protocol 2:

(i)
98L/408M;
160P/163S;
101N/103A;
99C/162C;
99C/162C/172V;
129Q/442I;
99A/161L;
99A/161L/442I;
161L/442I;
129M/442I;
99A/129M/161L/442I/444A;
98L/99A/129M/161L/442I/444A;
129M/161L/442I/444A;
98L/129M/161L/442I/444A; and/or
175I/203I; or

(ii)
160P/163S;
101N/103A;
99C/162C;
99C/162C/172V;
129Q/442I;
99A/161L;
99A/161L/442I;
161L/442I;
129M/442I;
99A/129M/161L/442I/444A;
98L/99A, 129M/161L 442I/444A;
129M/161L/442I/444A;
98L/129M/161L/442I/444A; and/or
175I/203I.

In various embodiments, the polypeptide includes 3, 4, 5, 6, 7, 8, 9, or more of the listed amino acid residues relative to SEQ ID NO:8 when aligned by protocol 1 or protocol 2. In a further embodiment, the polypeptides comprises the amino acid sequence at least 75%, 80%, 85%, 90%, 91%, 92%, 93%, 94%, 95%, 96%, 97%, 98%, or 99% identical to the amino acid selected from the group consisting of N8 mutants listed in Table 1 (SEQ ID NO:35-38), when aligned by protocol 1, wherein the polypeptide includes all of the residues listed in Table 1 for an individual N8 mutant listed in Table 1.

In various further embodiments, the polypeptides are at least 75%, 80%, 85%, 90%, 91%, 92%, 93%, 94%, 95%, 96%, 97%, 98%, or 99% identical to the N8 NA polypeptide of SEQ ID NO:8, wherein the non-naturally occurring polypeptide includes a 163E amino acid mutation relative to SEQ ID NO:8 when aligned by protocol 1, and further may optionally include a 194S mutation relative to SEQ ID NO:8 when aligned by protocol 1 or protocol 2.

In a ninth aspect, the NA polypeptides are:

(a) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 1, 2, 3, 4, 5, 6, 7, 8, 9, or all 10 of the following amino acid residues relative to SEQ ID NO:9 when aligned by protocol 1 or protocol 2:
94P, 95L, 100S, 126Q/M, 160V/A/I, 161V, 191Q, 198Y, 439S, 441V; or

(b) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 2, 3, 4, 5, 6, 7, 8, 9, 10, 11, 12, 13, or all 14 of the following amino acid residues relative to SEQ ID NO:9 when aligned by protocol 1 or protocol 2:
94P, 95L, 98N, 100S/A, 126Q/M, 152T, 158I, 160V/A/I/T, 161V, 191Q, 198Y, 200I, 439S, 441V.

In this ninth aspect, the N9 NA reference sequence is based on N9 reference strain:
A/Anhui/1-YK_RG39/2013 from H7N9.

(SEQ ID NO: 9)
MNPNQKILCTSATAIIGAIAVLIGIANLGLNIGLHLKPGCNCSHSQPETT
NTSQTIINNYYNETNITNIQMEERTSRNFNNLTKGLCTINSWHIYGKDNA
VRIGESSDVLVTREPYVSCDPDECRFYALSQGTTIRGKHSNGTIHDRSQY
RALISWPLSSPPTVYNSRVECIGWSSTSCHDGKSRMSICISGPNNNASAV
VWYNRRPVAEINTWARNILRTQESECVCHNGVCPVVFTDGSATGPADTRI
YYFKEGKILKWESLTGTAKHIEECSCYGERTGITCTCRDNWQGSNRPVIQ
IDPVAMTHTSQYICSPVLTDNPRPNDPNIGKCNDPYPGNNNNGVKGFSYL
DGANTWLGRTISTASRSGYEMLKVPNALTDDRSKPIQGQTIVLNADWSGY
SGSFMDYWAEGDCYRACFYVELIRGRPKEDKVWWTSNSIVSMCSSTEFLG
QWNWPDGAKIEYEL
(Head sequence is highlighted)

In one embodiment of this ninth aspect, the polypeptides are:

(a) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 1, 2, 3, 4, 5, 6, 7, 8, 9, or all 10 of the following amino acid residues relative to SEQ ID NO:9 when aligned by protocol 1 or protocol 2:
94P, 95L, 100S, 126Q/M, 160V/A/I, 161V, 191Q, 198Y, 439S, 441V.

In another embodiment, the polypeptides are:

(b) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 2, 3, 4, 5, 6, 7, 8, 9, 10, 11, 12, 13, or all 14 of the following ammo acid residues relative to SEQ ID NO:9 when aligned by protocol 1 or protocol 2:

94P, 95L, 98N, 100S/A, 126Q/M, 152T, 158I, 160V/A/I/T, 161V, 191Q, 198Y, 200I, 439S, 441V.

In various embodiments, the polypeptides include one or more of the following sets of amino acid residues relative to SEQ ID NO:9 when aligned by protocol 1 or protocol 2:
96C/159C;
96C/159C/441V;
96A/441A;
96A/126M/439I/441A;
95L/96A/126M/439I/441A;
126M/439I/441A;
95L/126M/439I/441A;
94P/191T;
98N/100A; and/or
94P/191T/200I.

In various embodiments, the polypeptides include 3, 4, 5, 6, 7, 8, 9 or more of the listed amino acid residues relative to SEQ ID NO:9 when aligned by protocol 1 or protocol 2. In a further embodiment, the polypeptides are is at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes a 160Q amino acid mutation relative to SEQ ID NO:9 when aligned by protocol 1, or may include a combination of 160Q/E and 172V amino acid residues relative to SEQ ID NO:9 when aligned by protocol 1 or protocol 2.

In another embodiment, the disclosure provides composition, comprising one or more of the non-naturally occurring polypeptides cf any embodiment or combination of embodiments disclosed herein linked to a scaffold. Linkage to scaffolds permits a plurality (2, 3, 4, 5, 6, 7, 8, 9, 10, or more) of the polypeptides to be displayed, which may enhance the immune response stimulated upon administration to a subject in need thereof, as described in the methods that follow. The compositions may comprise any scaffold suitable for an intended use. The one or more non-naturally occurring polypeptides may be linked covalently or non-covalently to such a scaffold. In one embodiment, the scaffold comprises a protein scaffold; in this embodiment, the one or more non-naturally occurring polypeptides may be covalently linked to the protein scaffold, including but not limited to by being expressed as a fusion protein with a protein component of the scaffold.

In another aspect the disclosure provides nucleic acids encoding the polypeptide or fusion protein of any embodiment or combination of embodiments of the disclosure. The nucleic acid sequence may comprise single stranded or double stranded RNA (such as an mRNA) or DN A in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limned to polyA sequences, modified Kozak sequences, and sequences encoding epitope tags, export signals, and secretory signals, nuclear localization signals, and plasma membrane localization signals. It will be apparent to those of skill in the art based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), non-naturally occurring polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the present disclosure provides pharmaceutical compositions, comprising one or more polypeptides, fusion proteins, compositions, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, fusion proteins, compositions, nucleic acids, expression vectors, and/or host cells may be the sole active agent in the pharmaceutical composition, or the composition or vaccine may further comprise one or more other active agents suitable for an intended use.

The polypeptides, fusion proteins, compositions, pharmaceutical compositions, nucleic acids, expression vectors, and/or host cells of the disclosure may be used for any suitable purpose, including but not limited to treat or limit development of influenza infections. For example, the polypeptides, fusion proteins, compositions, pharmaceutical compositions, nucleic acids, expression vectors, and/or host cells may be used to elicit on immune response to influenza virus. One type of immune response is a B-cell response, which results in the production of antibodies against the antigen that elicited the immune response. While all antibodies are capable of binding to the antigen which elicited the immune response that resulted in antibody production, preferred antibodies are those that provide broad heterosubtypic protection against influenza virus. Thus, the methods may elicit antibodies that bind to an influenza NA protein from a virus selected from the group consisting of influenza A viruses, influenza B viruses, and influenza C viruses. These methods may elicit antibodies that bind to an influenza NA protein from an influenza virus selected from the group consisting of H1, H2, H3, H4, H5, H6, H7, H8, H9, H10, H11, H12, H13, H14, H15, H16, H17, and H18 influenza A virus, and influenza B virus. The methods may elicit antibodies that bind to an influenza NA protein from a strain of influenza virus selected from the group consisting of influenza A/California/07/2009 (H1N1), A/Michigan/45/2015 (H1N1), A/New Caledonia/20/1999 (H1N1), A/WSN/1933 (H1N1), A/Brevig Mission/1/1918 (H1N1), A/Vietnam/1203/2004 (H5N1), A Wisconsin-67/2005 (H3N2), A/Swine/Missouri/2124514/2006 (H2N3), A/Red knot/Delaware Bay/310/2016 (H10N4), A/Shorebird/Del aware Bay/309/2016 (H10N5), A/chicken/Sichuan/NCJPL1/2014 (H5N6), A Netherlands/219/2003 (H7N7), A Jiangxi/IPB13b/2013 (H10N8), A/Anhui/1-YK_RG39/2013 (H7N9), B/Phuket/3073/2013, B/Colorado/06/2017 and antigenic variants thereof.

Protective antibodies elicited by methods of this disclosure can protect against viral infections by affecting any step in the life cycle of the virus. For example, protective antibodies may prevent an influenza virus from attaching to a cell, entering a cell, releasing viral ribonucleoproteins into the cytoplasm, forming new viral particles in the infected cell, and/or budding new viral particles from the infected host cell membrane. Antibodies elicited by the methods of this disclosure preferably prevent influenza virus from attaching to or entering the host cell, prevent fusion of viral membranes with endosomal membranes, or prevent release of newly formed virus from the infected host cell.

One aspect of this disclosure is a vaccine composition (vaccine) comprising any polypeptide, fusion protein, or composition disclosed herein, to protect subjects against infection by influenza virus. Vaccine of this disclosure can also contain other components such as adjuvants, buffers and the like. Exemplary adjuvants include aluminum phosphate, benzylalkonium chloride, ubenimex, and QS21; genetic adjuvants such as the IL-2 gene or fragments thereof, the granulocyte macrophage colony-stimulating factor (GM-CSF) gene or fragments thereof, the IL-18 gate or fragments thereof, the chemokine (C—C motif) ligand 21 (CCL21) gene or fragments thereof, the IL-6 gene or fragments thereof, CpG, LPS, TLR agonists, and other immune stimulatory genes, protein adjuvants such IL-2 or fragments thereof, the granulocyte macrophage colony-stimulating factor (GM-CSF) or fragments thereof, IL-18 or fragments thereof, the chemokine (C—C motif) ligand 21 (CCL21) or fragments thereof, IL-6 or fragments thereof, CpG, LPS, TLR agonists and other immune stimulatory cytokines or fragments thereof; lipid adjuvants such as cationic liposomes, N3 (cationic lipid), monophosphoryl lipid A (MPL1); other adjuvants including cholera toxin, enterotoxin, Fms-like tyrosine kinase-3 ligand (Flt-3L), bupivacaine, marcaine, and levamisole.

The vaccines of this disclosure may include immunogenic portions of more than one Type, Group, subtype, or strain of influenza virus. Such vaccine may comprise nanoparticles, each of which comprises immunogenic portions from NA proteins from more than one Type, Group, subtype, or strain of influenza virus. Such a vaccine is referred to as a multivalent vaccine. A multivalent vaccine can comprise immunogenic portions from as many influenza NA proteins as necessary to elicit production of an immune response sufficient to protect against a desired breadth of virus Types, Groups, subtypes, or strains. In one embodiment, the vaccine comprises immunogenic portions of NA proteins from at least two different influenza strains (i.e., a bivalent vaccine), or from at least three different influenza strains (i.e., a trivalent vaccine), or from at least four different influenza strains (i.e., a quadrivalent vaccine), or from at least five different influenza strains (i.e., a pentavalent vaccine). In one embodiment, the vaccine comprises immunogenic portions of NA proteins from at least six different influenza strains (hexavalent).

This disclosure provides methods of vaccinating a subject against influenza virus, the method comprising administering a polypeptides, compositions, pharmaceutical compositions, nucleic acids, expression vectors, and/or host cells to the subject such that an immune response against influenza virus is produced in the subject.

The subject may be any suitable subject, including but not limited to humans and other primates, including non-human primates such as chimpanzees and other apes and monkey species; farm animals such as cattle, sheep, pigs, seals, goats and horses; domestic mammals such as dogs and cats; laboratory animals including rodents such as mice, rats and guinea pigs; birds, including domestic, wiki and game birds such as chickens, turkeys and other gallinaceous birds, ducks, geese, and the like.

In the vaccination methods of this disclosure, the subject being vaccinated may have been exposed to influenza virus. As used herein, the term “exposed” indicate the subject has come in contact with a person or animal that is known to be infected with an influenza virus. Vaccines of this disclosure may be administered by any suitable technique, by means including, but not limited to, traditional syringes, needleless injection devices, or microprojectile bombardment gene guns. Suitable routes of administration include, but are not limited to, parenteral delivery, such as intramuscular, intradermal, subcutaneous, or intramedullary injections, as well as intrathecal, direct intraventricular, intravenous, intraperitoneal, intranasal, or intraocular injection.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### EXAMPLES

We provide sequences of recombinant NA proteins in which head domains comprising stabilizing mutations are connected to tetramerization domains. We initially found that many wild-type sequences of beta propeller head domains from certain NA subtypes adopted “open” conformations in which the head domains extended individually off of the stalk-like tetramerization domain, without forming the crystallographically observed “closed” tetramer. Constructs comprising the head domains from other NA subtypes formed closed tetramers naturally. Similar constructs from yet other subtypes formed mixtures of open and closed tetramers. We identified specific mutations at multiple locations in NA sequences that dictate the open or closed conformational state of NA tetramers, and used these mutations to generate closed, stabilized tetramers from multiple NA subtypes. We also converted a naturally closed NA tetramer to a fully open conformation by substituting residues that we identified as pivotal for tetramer closure, confirming the importance of these residues for determining the conformational state of NA. Monoclonal antibodies (mAbs) that bind across the interface of two neighboring protomers in the closed configuration bind better to closed tetramers than open tetramers. The mutations we provide may be useful for stabilizing other NA proteins that naturally form open tetramers when produced recombinantly.

Together, the disclosure provides mutations at defined locations in NA proteins that close the open structures of various NA tetramers. These stabilized NA structures can be used as vaccine antigens in either soluble form or when presented on scaffolds.

### Materials and Methods

### Protein Design and Expression

NA constructs were expressed by transient transfection in Expi293F cells (ThermoFisher Scientific) at a density of 2.5×10\hat{ }6 cells/ml using ExpiFectamine™ 293 Transfection Kit (ThermoFisher Scientific). The supernatants were harvested 5 days post transfection and centrifuged at 4000 rpm to remove cell debris. The culture supernatants were sterile filtered prior to purification by immobilized metal affinity chromatography (IMAC). Clarified supernatant was incubated for 2 h at room temperature with Ni Sepharose™ High Performance histidine-tagged protein purification resin (GE Healthcare) and separated through affinity chromatography. Bound protein was eluted with 300 mM imidazole, 50 mM Tris-HCl and 0.5 M NaCl. Eluted protein was further purified by size exclusion chromatography into phosphate-buffered saline (PBS) using a Superdex™ 200 Increase 10/300 column (GE Life Sciences).

### NA Antigenic Characterization

Several mAbs are known that can be used to assess the antigenicity or conformational state of NA. We used multiple mAbs for this purpose, including CD6, a mAb that binds across the interface of two protomers in the closed, crystallographically observed C4-symmetric configuration (Wan et al., Nat. Comms. 6:6114). We found that CD6 bound better to recombinant NA proteins that formed closed tetramers than NA proteins that formed open tetramers.

A fortéBio Octet™ HTX instrument was used to measure binding of NA proteins to antibodies that target several antigenic sites. All assays were performed in PBS supplemented with 1% bovine serum albumin (BSA) to minimize nonspecific interactions. The final volume for all solutions was 50 μl/well. Assays were performed at 30° C. in solid black 384-well plates. NA was loaded for 300-600 s on HIS1K tips, which were then dipped to capture mAbs for 600 s. mAbs were then allowed to dissociate for 300-600 s in PBS+1% BSA. Data analysis was carried out using Octet software, version 11. High capture levels of protein (same as reference proteins or higher) were part of the selection process for EM analysis. Binding of mAb to protein was the second step of the selection process for EM analysis.

### NA Activity Assay's

Neuraminidase activity was measured with the NA-Fluor Influenza Neuraminidase Assay Kit according to the manufacturer's protocol. Briefly, 50-100 μg/ml of protein was used as a start concentration and 2-fold dilutions were prepared in duplicate in a black 96-well, flat bottom plate for each protein sample. The wells in column 12 were left empty for controls. NA-Fluor Substrate was prepared according to the protocol and added to each well. Plates were incubated for 1 h at 37° C. and reactions were stopped with NA-Fluor Stop Solution. Plates were read using an excitation wavelength range of 350 nm to 365 nm and an emission wavelength range of 440 nm to 460 nm. Background control wells were subtracted for each protein serial dilution. Finally, protein dilutions were plotted versus relative fluorescence unit (RFU) values.

### EM Sample Preparation

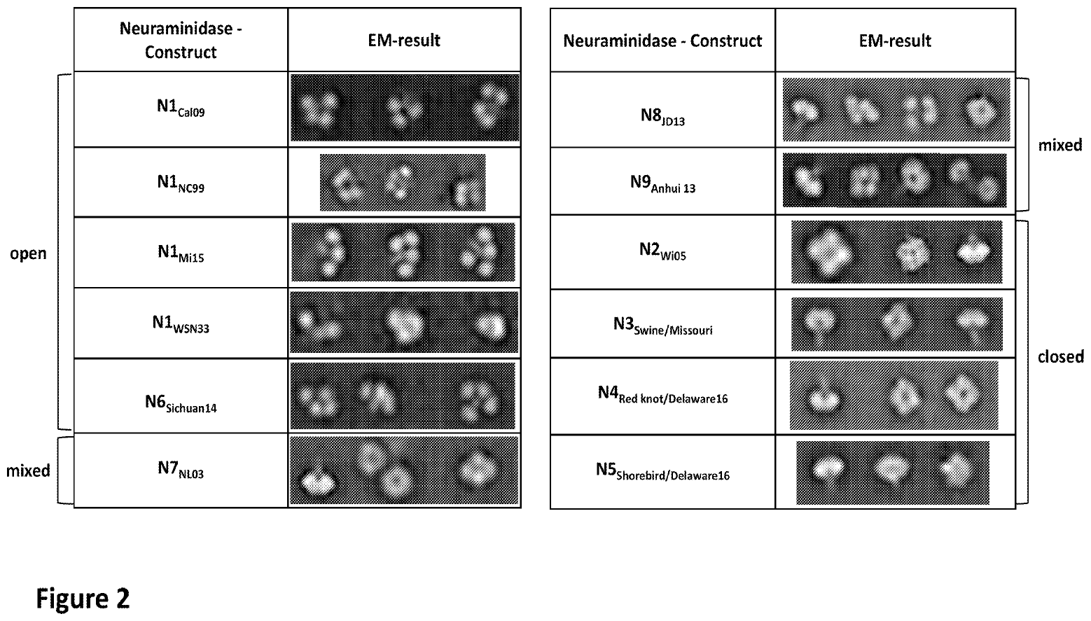

We used negative stain electron microscopy and particle averaging to assess whether the head domain of recombinant NA proteins adopted the open or closed structure. FIG. 2 shows representative examples of two-dimensional class averages of recombinant NA proteins that form open tetramers (N1_{Ca109}, N1_{NC99}, N1_{M115}, N1_{WSN33} and N6_{Schuan14}), mixtures of open and closed tetramers (N7_{NL03}, N8_{ID13}, and N9_{Anhuif3}), and closed tetramers (N2_{Wi0S}, N3_{Swine/Missouri}, N4_{Red knot/Delaware16}, and N5_{Shorebird/Delaware16}).

Proteins were diluted to a concentration of about 0.02 mg/ml with buffer containing 10 mM HEPES, pH 7.0, and 150 mM NaCl and adsorbed to a glow-discharged carbon-coated copper grid. The grid was washed with a drop of the same buffer three times and stained with 0.75% uranyl formate. Images were recorded at a nominal magnification of 100,000 (pixel size: 0.22 nm) using SerialEM software on an FEI Tecnai T20 electron microscope equipped with an FBI Eagle CCD camera and operated at 200 kV. Particles were selected from the micrographs automatically using in-house software (Yaroslav Tsybovsky, unpublished) and extracted into 128×128-pixel boxes. Reference-free 2D classification was performed using Relion 1.4.

### Results

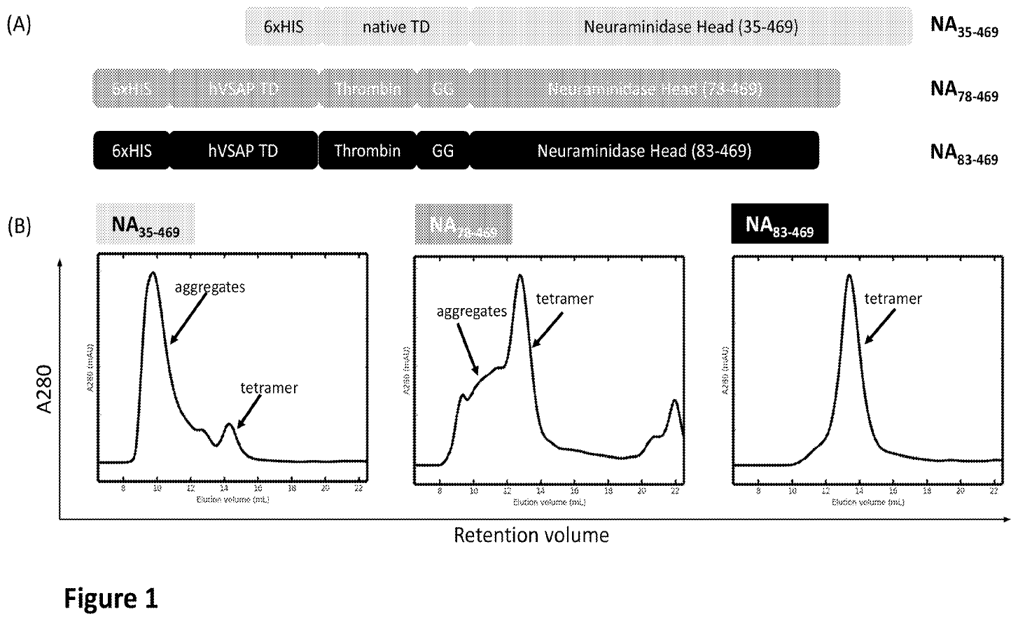

Referring to FIG. 1, three different recombinant NA designs were expressed (FIG. 1A), purified by IMAC, and examined by size exclusion chromatography (SEC). In the first recombinant NA configuration, an N-terminal 6× His tag was appended to residues 35-469 of A/California/07/2009 (H1N1) NA (SEQ ID NO: 1), comprising the native stalk and head domains. A second recombinant NA configuration was tested in which the cytosolic, transmembrane, and stalk domains of the wild-type NA (residues 1-77 in SEQ ID NO:1) were replaced by a 6× His-tag, an hVSAP domain to drive protein tetramerization (Xu et al., J. Virol. 82:10493-10501), a thrombin cleavage site, and a two-residue Gly-Gly linker. A third recombinant NA configuration was tested in which the cytosolic, transmembrane, and stalk domains of the wild-type NA (residues 1-82 in SEQ ID NO: 1) was replaced by a 6× His-tag, an hVSAP domain, a thrombin cleavage site, and a two-residue Gly-Gly linker. Recombinant NAs in which the head domain started at position 83 showed homogeneous SEC profiles (FIG. 1B) with a major peak corresponding to the estimated molecular weight of NA tetramers, with minimal aggregation. Subsequent constructs were designed with head domains starting at position 83.

Referring to FIG. 2, purified recombinant NA proteins from a number of subtypes were characterized structurally by negative stain EM. Representative NAs from the N2, N3, N4, N5 subtypes formed closed tetrameric structures in which the head domain resembled the C4-symmetric structure classically observed by X-ray crystallography. Representative NAs from the N1 and N6 subtypes formed open tetramers in which the head domains did not form a single, compact structure. Representative NAs from the N7, N8, and N9 subtypes formed mixtures of open and closed tetramers.

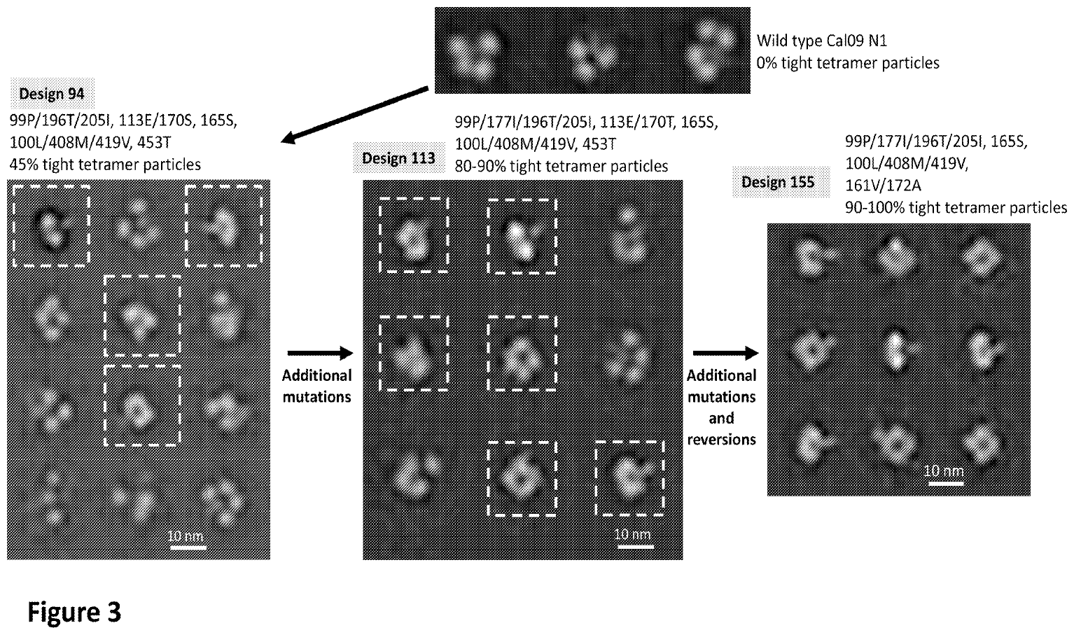

Referring to FIG. 3, in one non-limiting example, we designed a series of recombinant NA mutants based on the wild-type A/California/07/2009 (H1N1) NA sequence that resulted in a protein that formed a closed tetramer. Introducing ten mutations into A/California/07/2009 (H1N1) NA (construct name 94_N1-Cal09_danfav2 in Table 1; SEQ ID NO:79) resulted in 45% closed tetramers. Additional mutations and a reverse mutation result in a protein (construct name 155_N1-Cal09-c130_T453V in Table 1; SEQ ID NO:13) that forms 90-100% closed recombinant NA tetramers. The 99P, 177I, 196T and 205I mutations provide improved hydrophobic packing at the infer-protomeric interface. The 165S mutation helps stabilize the closed conformation of a loop that participates in the inter-protomeric interface. The 161V and 172A mutations remove a cysteine and optimize packing, which improves expression. The 100L, 408M and 419V mutations improve expression by removing polar residues in a region of the protein that is partially hidden from solvent.

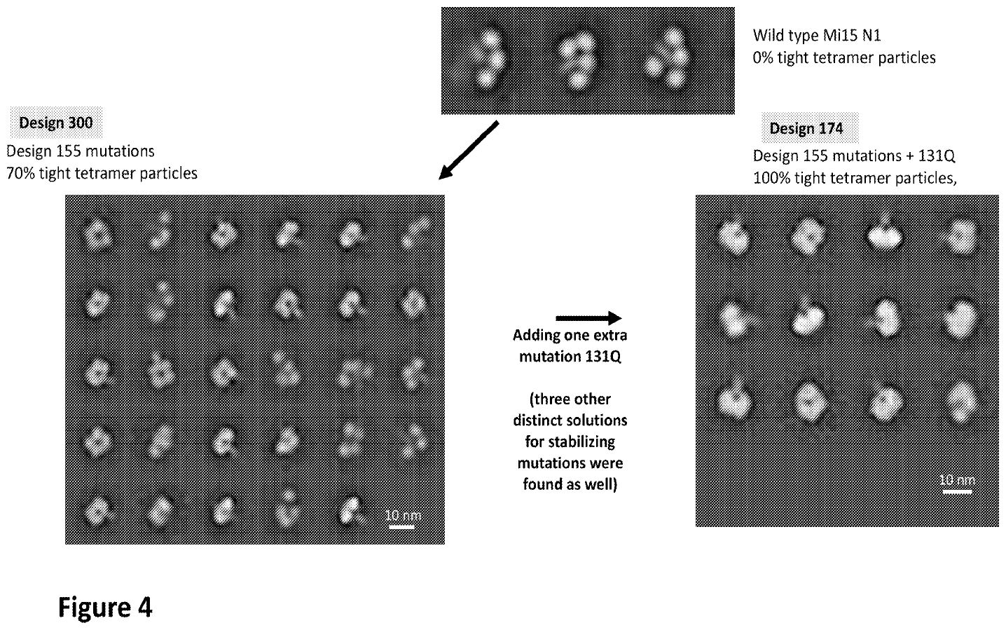

Referring to FIG. 4, in another non-limiting example, stabilizing mutations introduced into the sequence of A/Michigan/45/2015 (H1N1) NA, which forms an open tetramer when the wild-type sequence for the head domain is used, result in a protein (construct name 174_N1-Mi15_c155_T131Q in Table 1; SEQ ID NO: 18) that forms 100% closed tetramers. The 131Q mutation optimizes packing and helps stabilize the closed conformation of a loop that participates in the inter-protomeric interlace.

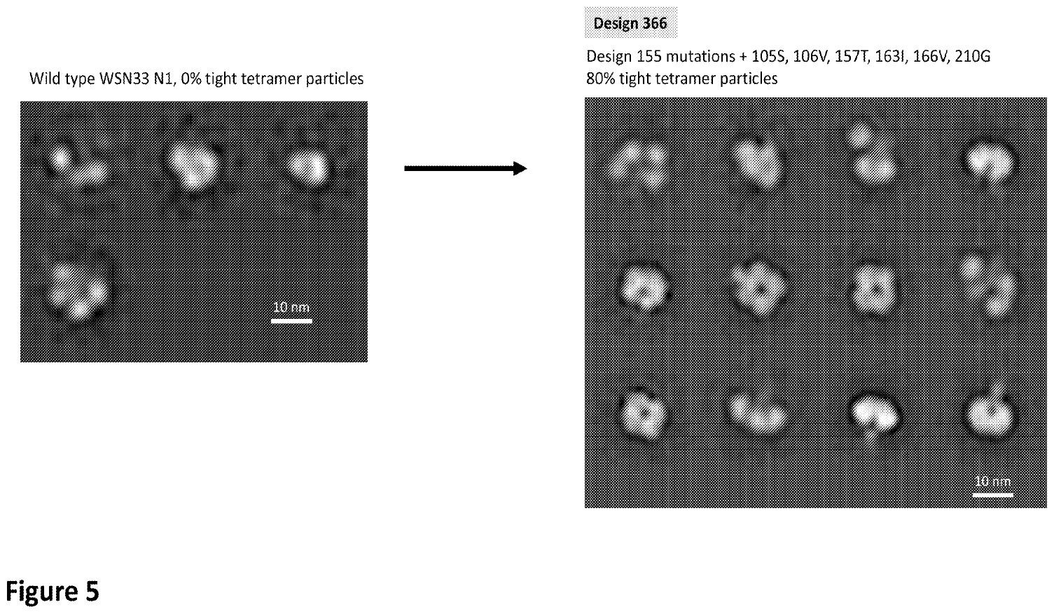

Referring to FIG. 5, in another non-limiting example, stabilizing mutations introduced into the sequence of A/WSN/1933 (H1N 1) NA, which forms an open tetramer when the wild-type sequence for the head domain is used, result in a protein (construct name 366_N1-WSN33_c155_G105S_I106V_A157T_V163I_A166V_R210G in Table 1; SEQ ID NO:43) that forms 80% closed tetramers. The 157T and 166V mutations provide improved hydrophobic packing at the inter-protomeric interface. The 210G mutation removes electrostatic repulsion at the inter-protomeric interface. The 105S, 106V and 163I mutations help optimize packing to stabilize the closed conformation of a loop that participates in the inter-protomeric interface.

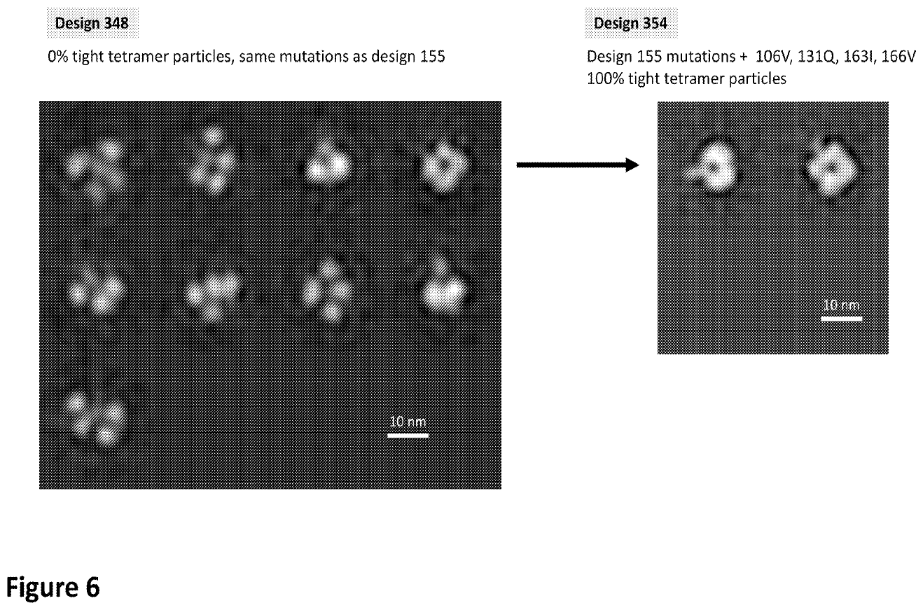

Referring to FIG. 6, in another non-limiting example, stabilizing mutations introduced into the sequence of A/Vietnam/1203/04 (H5N1) NA, which forms an open tetramer when 10 stabilizing mutations originally identified in A/California/07/2009 (H1N1) NA are used, result in a protein bearing 14 mutations (construct name 354_N1-VN04_c155_I106V_T131Q_V163I_A166V in Table 1; SEQ ID NO:40) that forms 100% closed tetramers. The 106V, 131Q and 163I mutations help optimize packing to stabilize the closed conformation of a loop that participates in the inter-protomeric interface. The 166 V mutation provides improved hydrophobic packing at the inter-protomeric interface.

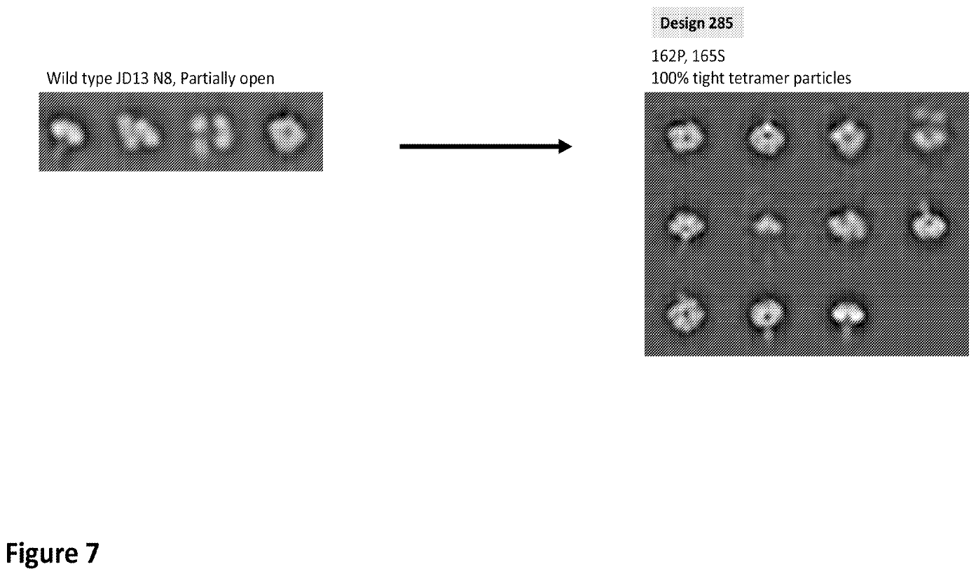

Referring to FIG. 7, two mutations introduced into the recombinant A/Jiangxi-Donghu/346-2/2013 (H10N8) N8 NA (construct name 285_N8-Jiangxi-Donghu2013_E162P-Q165S in Table 1; SEQ ID NO:36) resulted in the formation of 100% closed tetramers. The 162P and 165S mutation help stabilize the closed conformation of a loop that participates in the inter-protomeric interface.

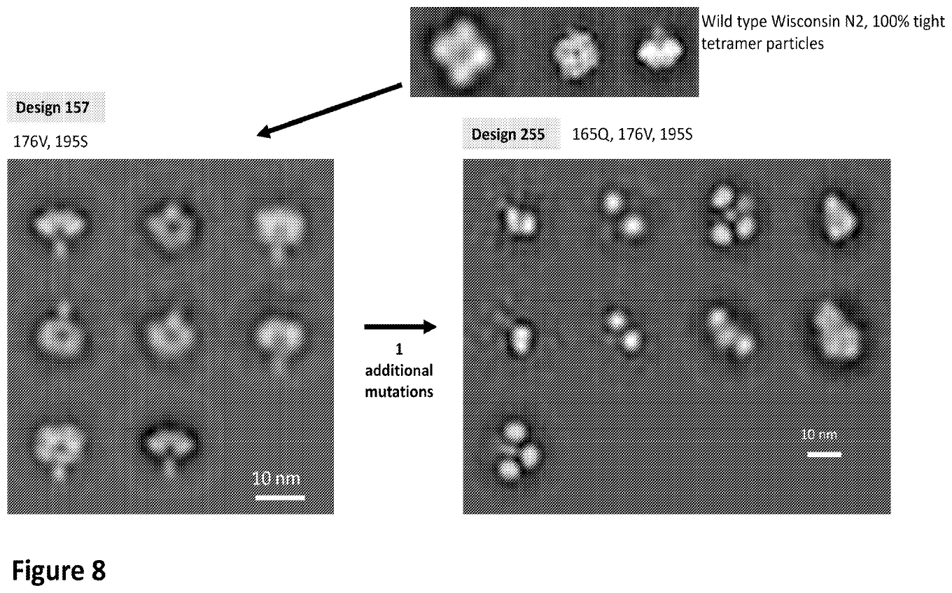

Referring to FIG. 8, we introduced substitutions at positions that are critical for stabilizing the closed tetrameric structure of NA into a naturally closed recombinant NA to open up the closed, compact structure. Three mutations at positions that had been identified to stabilize open tetramers resulted in the formation of exclusively open tetramers of the NA from A/Wisconsin/67/2005 (H3N2) (construct name 255_N2-Wis05_V165Q_I176V_T 195S in Table 1; SEQ ID NO:34). The 176V and 195S mutations decrease the amount of inter-protomeric hydrophobic packing that is natively present. The 165Q mutation allows for increased flexibility of a loop that participates *n the inter-protomeric interface

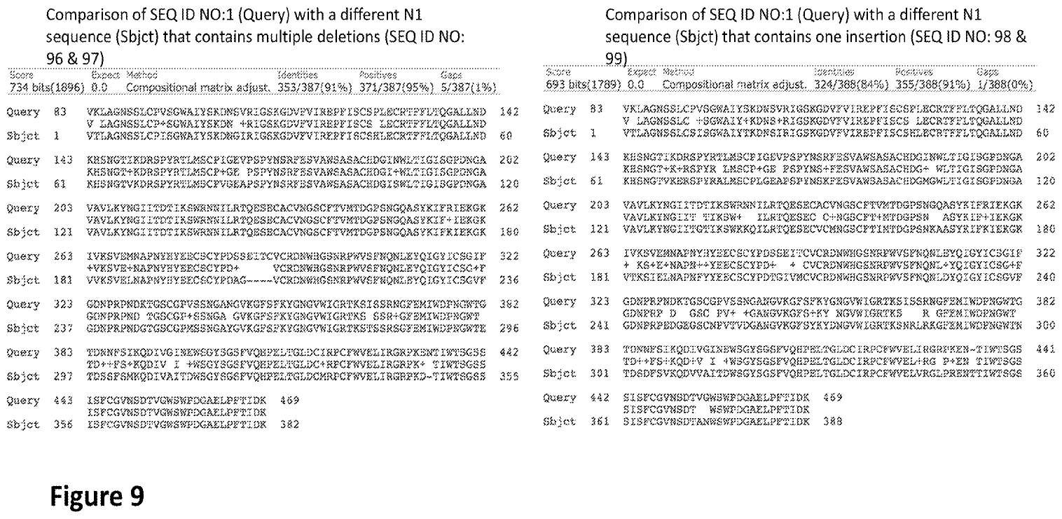

Referring to FIG. 9, a Basic Local Alignment Search fool protein (BLASTp) alignment between reference sequences and any arbitrary sequence of the same subtype allows for identification of positions for the described mutations in any NA sequence. Sequence positions in arbitrary NA sequences are identified based on alignment to corresponding positions in reference sequences. Many sequence alignment tools are known to those of skill in the art and can be used to align arbitrary NA sequences to the provided reference sequences. Here we provide protocols for aligning sequences using the online BLASTp tool provided by the National Institute of Health (protocol 1) as well as a standalone executable version of the BLAST software that can be run locally on any computer (protocol 2).

Overall, we identified mutations that result in recombinant NA proteins that adopt closed, C4-symmetric, or open, non-symmetric conformations. Mutations that fill cavities in NA are helpful for tetramer closure. In some non-limiting examples, engineered disulfide bonds are helpful for tetramer closure. Certain amino acid positions, including but not limited to position 165, appear most relevant for dictating the open or closed conformational state of NA tetramers. In addition, other mutations substantially improve overall protein expression levels.

TABLE 1

| | | Mutations | Mutations highlighted in | % |
|---|---|---|---|---|
| Name | Sequence | included | uppercase | Closure |
| 94_N1- | VKLAGNSSLCPVSGWAPLSKDNSVRTG | N1 numbering: | vklagnsslcpvsgwaPLskdnsvrig | 45% |
| Ca109_d [symbol] n | SKGEVFVIREPFLSCSPLXCRTFFLTQ | 99P/196T/205I, | skgEvfyirepfiscsplscrtffltq | |
| fav2 | GALLNDKHSNGTLKDRSPYRTLMSCPI | 113E/170S, | gallndkhsngtikdrspystlmscpi | |
| | GSVPSPSNBRFESVAWSASACHDGINW | 165S, | gSvpspSnsrfesvawsasachdginw | |
| | LTIGITGPGNSAVAILKYNGIITDTIK | 1 [symbol] L/408M/419V, | ltigiTgpdngavailkyagiitdtik | |
| | SWRNMILRTQESECACVNGSCFTVMTU | 453T | swrnnilrtqesecacvngseftvmtd | |
| | GPSNGDASYKIFRIEKGKIVKSVEMNA | | gpanggasykifriekgkivksvemna | |
| | PNYHYPECDCYPDSSEITCVCRDNWNG | | pnyhyeecscypdnseitcvcrdnwhg | |
| | SNRPWVSPNQNLEYQIGYIGSGIFGDN | | snrpwvsfnqnleyglgyicsglfgdn | |
| | PR [symbol] ND [symbol] GSCCPVSENGANGVKGFEPK | | prpndktgasgpvssngangvkgfsfk | |
| | YGNGVWIGRTKSISSRNGFEMIWDPNG | | ygngvwlgrtkslssrngfemiwdpng | |
| | WTGTDNNFSIKQDIVGINEWSGYSGSF | | wtgtdnnfsikqdivginewsgysgsf | |
| | VMHPELTGLDCIVPCPWVELIESRPKE | | vMkpeltgldciVpcfwvelirgrpke | |
| | NTIWTSGSSISFCGVNS [symbol] TTGWSWPDG | | ntiwtsgssisfcgvnsdtTgwswpdg | |
| | AELPFTIDK (SEQ ID NO: 79) | | aslpftldk (SEQ ID NO: 79) | |
| 112_N1- | VKLAGNSNLCPVSGWAPYSKDNSVRIG | N1 numbering: | vklagnsnlcpvsgwapyskdnsvrig | 40% |
| Ca109_d [symbol] n | SKGEVFVIREPFISCSPLECRTFFLTQ | 99P/196T/205I, | skgEvfvirepfiscsplecrtffltq | |
| fav2_no- | GALLNDKHSNGTIKDRSPYRTLMSCPL | 113E/170S, | gallndkhsngtikdrspyrtlmscpl | |
| space-5 | GSVPSPSNSRFESVAWSASACHDGINW | 165S, 453T | gSvpspSnsrfesvawsasachdginw | |
| | LTIGITGPDNGAVATLKYNGIITDTIK | | ltigiTgpdngavatlkyngiitdtik | |
| | SWRNNILRTQESECACVNGSCFTVMTD | | swrnnilrtqesecacvngscftvmtd | |
| | GPSNGQASYKIFRIERGKIVKSVEMNA | | gpsngqasykifriergkivksvemna | |
| | PNYHYEECSCYPDSSEITCVCRDNWHG | | pnyhyeecscypdsseitcvc [symbol] nwhg | |
| | SNRPWVSFNQNLEYQIGYICSSIFGDN | | snrpwvsfnqnleyqigyicssifgdn | |
| | PRPGDKTGSCGPVSSNGANGVKGFSFK | | prpgdktgscgpvssngangvkgfsfk | |
| | YGNSVWIGRTKSISSRNGFEMIWDPNG | | ygnsvwigrtksissrngfemiwdpng | |
| | WTGTDNNFSIKQDIVGINEWSGYSGSF | | wtgtdnnfsikqdivginewsgysgsf | |
| | VQHPELTGLDCIRPCPWVELIRGRPKE | | vqhpeltgldcirpcpwvelirgrpke | |
| | NTIWTSGSSILFCGVNSDTTGWSWPDG | | ntiwtsgssilfcgvnsdtTgwswpdg | |
| | ACLPCTIDK (SEQ ID NO: 80) | | aclpctidk (SEQ ID NO: 80) | |
| 113_N1- | VKLAGNSSLCPVSGWAPLSKDNSVRIG | N1 numbering: | vklagnsslcpvsgwaPLskdnsvrig | 80-90% |
| Ca109_DF2 | SKGEVFVIREPFISCSPLECRTFFLTQ | 99P/177I/196T/ | skgEvfvirepfiscsplecrtffltq | |
| TI | GALLNDKHSNGTIKDRSPYRTLMSCPL | 205I, | gallndkhsngtikdrspyrtlmscpl | |
| | GSVPSPTNSRFESIAWSASACHDSINX | 113E/170T, | gSvpspTnsrfesIawsasachdsinx | |
| | LTIGITGPDNGAVAILKYNGLITDTIK | 165S, | ItigiTgpdngavaIlkynglitdtik | |
| | SWRNPNILRTQESECACVNGSCFTVMD | 1 [symbol] L/408M/419V, | swrnpnilrtqesecacvngscftvmd | |
| | GPSNGQASYKIFRIEKGKIVKSVEMNA | 453T | gpsngqasykifriekgkivksvemna | |
| | PNYHYEECSCYPDSSEITCVCRDNWHG | | pnyhyeecscypdsseitcvcrdnwhg | |
| | SNRPWVSPNQNLEYQIGYICSGIFGDN | | snrpwvspnqnleyqigyicsgifgdn | |
| | PRPGDKTGSCGPVSSNGANGVKGFSFK | | prpgdktgscgpvssngangvkgfsfk | |
| | YGNGVXIGRTKSISSRNGFEMIWDPNG | | ygngvxigrtksissrngfemiwdpng | |
| | WTGTDNNFSIKQDIVGINEWSGYSGSF | | wtgtdnnfsikqdivginewsgysgsf | |
| | VMNPELTGLDCIVPCFWVELIRGRPKE | | vMnpeltgldciVpcfwvelirgrpke | |
| | NTIWTSGSSISFCGVNSDTTGWSWPDG | | ntiwtsgssisfcgvnsdtTgwswpdg | |
| | AELPFTIDK (SEQ ID NO: 81) | | aelpftidk (SEQ ID NO: 81) | |
| 114_N1- | VKLAGNSSLCPVSGWAPLSKDNSVRIG | N1 numbering: | vklagnsslcpvsgwaPLskdnsvrig | 100% |
| Ca109_DF2 | SKGEVFVIREPFISCSPLECRTFFLTQ | 99P/177I/196T/ | skgEvfvirepfiscsplecrtffltq | |
| TI_T170S | GALLNDKESNGTIKDRSPYRTLMSCPI | 205I, | gallndkesngtikdrspyrtlmscpi | |
| | GSVPSPSNSRFESIAWSASACHDGINX | 113E/170S, | gSvpspSnsrfesiawsasachdginx | |
| | LTISITGPDNGAVAILKYNGLITDTIK | 165S, | ltisiTgpdngavaIlkynglitdtik | |
| | SWKNNILRTQESECACVNGSCFTVMTD | 1 [symbol] L/408M/419V, | swknnilrtqesecacvngscftvmtd | |
| | GPSNGQASYKIFRIEKGKIVKSVSMNA | 453T | gpsngqasykifriekgkivksvsmna | |
| | PNYHYESCSCYPDSSEITCVCRDNWHG | | pnyhyescscypdsseitcvcrdnwhg | |
| | SNRPWVSFNQSLEYQIGYICSGIFGDN | | snrpwvsfnqsleyqigyicsgifgdn | |
| | PRPNDKTGSCGPVSSNGSNGVKGFSFK | | prpndktgscgpvssngsngvkgfsfk | |
| | YGNSVWIGRTKSISSRNGFEMIWDPNG | | ygnsvwigrtksissrngfemiwdpng | |
| | WTGTDNNTSIKQDIVGINEWSGYSGSF | | wtgtdnntsikqdivginewsgysgsf | |
| | VMHPELTGIDCIVPCTWVELIRGRPKE | | vMhpeltgidciVpctwvelirgrpke | |
| | NTIWTSGSSISFCGVNSDTTGWSWPDG | | ntiwtsgssisfcgvnsdttgwswpdg | |
| | AELPFTIDK (SEQ ID NO: 82) | | aelpftidk (SEQ ID NO: 82) | |
| 127_N1- | VKLAGNSSLCPVSGWAPYSKDNSVRIG | N1 numbering: | vklagnsslcpvsgwaPyskdnsvrig | 30-40% |
| Ca109_DF2 | SKGEVFVIREPFISCEPLECRTFFLTD | [symbol] | skgEvfvirepfisceplecrtffltd | |
| TI_CysKO_no- | GALLNDKHSNSTIKDRSPYRTIMEVPL | [symbol] | gallndkhsnstikdrspyrtimeVpl | |
| space-B | GSVPSTNARFERSIAWSASACHDGINW | 113E/170T, | gSvpsTnArfersiawsasachdginw | |
| | LTIGITGPDNGAVALLKYNSIITDTIK | [symbol] | ltigiTgpdngavallkynsiitdtik | |
| | SWRNNILRTQESECACVNGSCFTVMTD | [symbol] | swrnnilrtqesecacvngscftvmtd | |
| | GPSNGQASYKIFRIEKGKIVKSYEMNA | | gpsngqasykifriekgkivksyemna | |
| | PNYHYEECSCYPDSSEITCVCRDNWHG | | pnyhyeecscypdsseitcvcrdnwhg | |
| | SNRPWVSFNWNLPYQIGYICSGIPGDN | | snrpwvsfnwnlpyqigyicsgipgdn | |
| | PRPNDKTGSCGPVSSNGANGVKSPSFK | | prpndktgscgpvssngangvkspsfk | |
| | YGNGVWIGRTKSISSRNGFEMIWDPNG | | ygngvwigrtksissrngfemiwdpng | |
| | WTGTDNNFSIKQDIVGINEWSGYSGSF | | wtgtdnnfsikqdivginewsgysgsf | |
| | VQHPELTGLDCIRPCFWVELIRGRPKE | | vqhpeltgldcirpcfwvelirgrpke | |
| | NTIWTSGSSISFCGVNSDTTGWSWPDG | | ntiwtsgssisfcgvnsdtTgwswpdg | |
| | AELPFTIDK (SEQ ID NO: 83) | | aelpftidk (SEQ ID NO: 83) | |
| 128_N1- | VKLAGNSSLCPVSGMAPLSKDNSVRIG | N1 numbering: | vklagnsslcpvsgmaPLskdnsvrig | 90-100% |
| Ca109_DF2 | SKGEVFVIRPRFISCSPLECRTFFLTQ | [symbol] | skgevfvirprfiscsplecrtffltq | |
| TI_CysKO_T453V | GALLNDK [symbol] SNGTIKDRSPYRTLMNVP [symbol] | [symbol] | gallndk [symbol] sngtikdrspyrtlmnVp [symbol] | |
| | GSVPSPTNAKFESIAWSASACHDGINW | 113E/170T, | gSvpspTnAkfesiawsasachdginw | |
| | LTIGITGPDNGAVAILKYNGIITDTIK | 165S, | ltigiTgpdngavailkyngiitdtik | |
| | SWRNNILRTQESCCACVNGSCFTVMTD | [symbol] | swrnnilrtqesccacvngscftvmtd | |
| | GPSNGQASYKIFRIEKGKIVKSVEMNA | [symbol] | gpsngqasykifriekgkivksvemna | |
| | PNYHYEECSCYPDSSEITCVCRDNWHG | | pnyhyeecscypdsseitcvcrdnwhg | |
| | SNFPWVSFNQNLEYQIGYICSGIFGDN | | snfpwvsfnqnleyqigyicsgifgdn | |
| | PRPNDKTGSCGDVSSNGANGVKGFSFK | | prpndktgscgdvssngangvkgfsfk | |
| | YGNGVWIGPTKSISSPNGFEMIWDPNG | | ygngvwigptksisspngfemiwdpng | |
| | WTGTDNNFSIKQDIVGINEWSGYSGSF | | wtgtdnnfsikqdivginewsgysgsf | |
| | VMHPELTGLDCIVPCFWVELIRGKPKE | | vMhpeltgldcivpcfwvelirgkpke | |
| | NTIWTSGSSISFCGVNSDTFGWSWPDG | | ntiwtsgssisfcgvnsdt [symbol] gwswpdg | |
| | AELPFTIDK (SEQ ID NO: 84) | | aelpftidk (SEQ ID NO: 84) | |
| 130_N1- | VKLAGNSSLCPVSGWAPLSKDNSVRIG | N1 numbering: | vklagnsslcpvsgwaPLskdnsvrig | 90-100% |
| Ca109_DF2 | SKGDVFVIREPFISCSPLECRTPFLTQ | [symbol] | skgdvfvirepfiscsplecrtpfltq | |
| TI_CysKO_ | GALLNDKNSNGTLKDRSPYRTLMSVP [symbol] | [symbol] | gallndknsngtlkdrspyrtlmsVp [symbol] | |
| [symbol] | GSVPSPYNARFEEIAWSASACHDGINW | 100L/408M/419V, | gSvpspynArfesIawsasachdginw | |
| [symbol] | LTIGITGPDNGAVA [symbol] LKYNG [symbol] TDIK | 453T, | ltigiTgpdngavaTlkyng [symbol] dtik | |
| | SWRNMILKTQESECACVNGSCFTVMTD | [symbol] | swrnmilktqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWHG | | pnyhyeecscypdsseitcvcrdnwhg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINEWSGYSGSP | | wtgtdnnfsikqdivginewsgysgsp | |
| | VMHPELTGLDCIVPCFWVEIIRGRPKE | | vMhpeltgldciVpcfwveiirgrpke | |
| | STIWTSGSSISFCGVNSDTTGWSWPDG | | stiwtsgssisfcgvnsdtTgwswpdg | |
| | AELPFTIDK (SEQ ID NO: 85) | | aelpftidk (SEQ ID NO: 85) | |
| 131_N1- | VKLAGNSSLCPVSGWAPYSKDNSVRIG | N1 numbering: | vklagnsslcpvsgwaPyskdnsvrig | 90-100% |
| Ca109_DF2 | SKGDVFVIREPFISCSPLECKTFFLTQ | [symbol] | skgdvfvirepfiscsplecktffltq | |
| TI_CysKO_no- | GALINDKHSNGTIKDRSPYRTLMSVPI | [symbol] | galindkhsngtikdrspyrtlmsVpi | |
| space- | GSVPSPYNDRFESISWSASACHDGINW | 453T, | gSvpspyndrfesiswsasachdginw | |
| B_E113D_T170Y | LTIGITGPDNGAVAILKYNGIITDTIK | [symbol] | ltigiTgpdngavailkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYESCSCYPFSSEITCVCRDNWHG | | pnyhyescscypfsseitcvcrdnwhg | |
| | SNRPWVSFNQNLEYQIGYICSGIFGDN | | snrpwvsfnqnleyqigyicsgifgdn | |
| | PRPNDKTGSCGPVSSNGANGVKGPSFK | | prpndktgscgpvssngangvkgpsfk | |
| | YGNYVWIGRTKSISSRNGFEMIWDPNG | | ygnyvwigrtksissrngfemiwdpng | |
| | WTGTDNNFSIKGDIVGINEWSGYSGSF | | wtgtdnnfsikgdivginewsgysgsf | |
| | VQHPELTFLDCIRFCFWVELIRGRPKE | | vqhpeltfldcirfcfwvelirgrpke | |
| | NTIWTSGSSISFCGVNSDTTGWSWPDG | | ntiwtsgssisfcgvnsdtTgwswpdg | |
| | AELPFTIDK (SEQ ID NO: 86) | | aelpftidk (SEQ ID NO: 86) | |
| 134_N1- | VKLAGNSSLCPVSGWAPLSKDNSVRIG | N1 numbering: | vklagnsslcpvsgwaPLskdnsvrig | [symbol] |
| Ca109_DF2 | SKGGVKVIREPFISCSPLECRTFFLTG | [symbol] | skggvkvirepfiscsplecrtffltg | |
| TI_CysKO_ | GALLNDKHSNGTIKDRSPYRTLMSVPI | [symbol] | gallndkhsngtikdrspyrtlmsVpi | |
| [symbol] | GEVPSPYNARFESTAWSASACHDGINW | 100L/408M/419V, | gevpspynArfestawsasachdginw | |
| [symbol] | LTIGITGPDNGAVAILKYNGIITDTIK | 161V/172A | ltigiTgpdngavailkyngiitdtik | |
| [symbol] | SWRNNILRTQESECACVNGSCFTVMTD | | swrnnilrtqesecacvngscftvmtd | |
| | GPSNSQASYKIFRIRKGKIVKSVEMNA | | gpsnsqasykifrirkgkivksvemna | |
| | PNYHYEECSCYPDSSEITCVCHDNWHG | | pnyhyeecscypdsseitcvchdnwhg | |
| | SNRPWVRFNQNLEYQIGYICSGIFGDN | | snrpwvrfnqnleyqigyicsgifgdn | |
| | PRPNDKTFSCFPVSSNGANGVKGFSFK | | prpndktfscfpvssngangvkgfsfk | |
| | YGNGVWIGPTKSISSPNGFEMIWDPNG | | ygngvwigptksisspngfemiwdpng | |
| | WTGTDNNFSIKQDIVGINEWSGYSGSF | | wtgtdnnfsikqdivginewsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisgcgvnsdfvgwswpdg | |
| | AELPFTIDK (SEQ ID NO: 10) | | aelpftidk (SEQ ID NO: 10) | |
| 140_N1- | VKLAGNSSLCPVSGWAPLSKDNSVRIG | N1 numbering: | vklagnsslcpvsgwaPLskdnsvrig | 10-20% |
| M115_DF2TI | SKGEVFVIREPFISCSPLECPFFPLTQ | 99P/177 [symbol] /196T/ | skgEvfvirepfiscsplecptffltq | |
| | GALLNDKHSNGTIKDRSPYRTLMSCPI | 205I, | gallndkhsngtikdrspyrtlmscpi | |
| | GSVPSPTNSRFESIAWSASACHDGINW | 113E/170T, | gSvpspTnsrfesTawsasachdginw | |
| | LTIGITGPDNGAVAILKYNGIITDTIK | 165S, | ltigiTgpdngavaIlkyngiitdtik | |
| | SWRNNILRTQESECACVNGSCFTIMTD | 100L/408M/419V, | swrnnilrtqesecacvngscftimtd | |
| | GPSDGQASYKIFRIRKGKTIKSVEMKA | 453T | gpsdgqasykifrirkgktiksvemka | |
| | PNYHYEECSCYPDSSEITCVCNDNWHG | | pnyhyeecscypdsseitcvcndnwhg | |
| | SNRPWVRFNQNLEYQIGYICSGIFGDN | | snrpwvrfnqnleyqigyicsgifgdn | |
| | PRPNDKTFSCFPVSSNGANGVKGFSFK | | prpndktfscfpvssngangvkgfsfk | |
| | YGNGVWIGPTKSISSPNGFEMIWDPNG | | ygngvwigptksisspngfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSISFCGVNSDTVGWSWPDG | | ntiwtsgssisfcgvnsdTvgwswpdg | |
| | AELPFTIDK (SEQ ID NO: 11) | | aelpftidk (SEQ ID NO: 11) | |
| 141_N1- | VKLAGNSSLCPVSGWAPYSKDNSVRIG | N1 numbering: | vklagnsslcpvsgwaPyskdnsvrig | 40% |
| M115_DF2TI_no- | SKGEVFVIREPFISCSPLECPFFPLTQ | 99P/177 [symbol] /196T/ | skgEvfvirepfiscsplecpffpltq | |
| space- [symbol] | GALLNDKHSNGTIKDRSPYRTLMSCPI | 205I, | gallndkhsngtikdrspyrtlmscpi | |
| | GSVPSPTNSRFESIAWSASACHDGINW | 113E/170T, | gSvpspTnsrfesIawsasachdginw | |
| | LTIGITGPDNGAVAILKYNGIITDTIK | 165S, 453T | ltigiTgpdngavaIlkyngiitdtik | |
| | SWRNNILRTQESECACVNGSCFTIMTD | | swrnnilrtqesecacvngscftimtd | |
| | GPSDGQASYKIFRIRKGKTIKSVEMKA | | gpsdgqasykifrirkgktiksvemka | |
| | PNYHYEECSCYPDSSEITCVCRDNWHG | | pnyhyeecscypdsseitcvcrdnwhg | |
| | SNRPWVSFNQNLSYQMGYICSGVFGDN | | snrpwvsfnqnlsyqmgyicsgvfgdn | |
| | PRPNDKTGSCGFVSSNGANGVKGFSFK | | prpndktgscgfvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VQHPELTGLDCIRPCFWVELIRGRPEE | | vqhpeltgldcirpcfwvelirgrpee | |
| | NTIWTSGSSISFCGVNSDT [symbol] GWSWPDG | | ntiwtsgssisfcgvnsdt [symbol] gwswpdg | |
| | AELPFTIDK (SEQ ID NO: 12) | | aelpftidk (SEQ ID NO: 12) | |
| 155_N1- | VKLAGNSSLCPVSGWAPLSKDNSVRIG | N1 numbering: | vklagnsslcpvsgwaPLskdnsvrig | 90-100% |
| Ca109- | SKGDVFVIREPFISCSPLECPFFPLTQ | 99P/177 [symbol] /196T/ | skgdvfvirepfiscsplecpffpltq | |
| C130_T453V | GALLNDKHSNGTIKDRSPYRTLMSVPI | [symbol] | gallndkhsngtikdrspyrtlmsVpi | |
| | GSVPSPYNARFESIAWSASACHDGINW | 100L/408M/419V, | gSvpspynArfesIawsasachdginw | |
| | LTIGITGPDNGAVAILKYNGIITDTIK | [symbol] | ltigiTgpdngavailkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWHG | | pnyhyeecscypdsseitcvcrdnwhg | |
| | SNRPWVSFNQNLSYQIGYICSGIFGDN | | snrpwvsfnqnlsyqigyicsgifgdn | |
| | PRPNDKTGSCGFVSSNGANGVKGFSFK | | prpndktgscgfvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTIDK (SEQ ID NO: 13) | | aelpftidk (SEQ ID NO: 13) | |
| [symbol] | VKLAGNSSLCPVSGWAPLSKDNSVRIG | N1 numbering: | vklagnsslcpvsgwaPLskdnsvrig | 70% |
| c155 | SKGDVFVIREPFISCSPLECRTFFLTQ | 99P/177I/196T/ | skgdvfvirepfiscsplecrtffltq | |
| | GALLNDKHSNGTIKDRSPYRTLMSVPI | 205I, 165S, | gallndkhsngtikdrspyrtlmsVpi | |
| | GSVPSPYNARFESIAWSASACHDGINW | 100L/408M/419V, | gSvpspynArfesIawsasachdginw | |
| | LTIGITGPDNGAVAILKYNG [symbol] ITDTIK | [symbol] | ltigiTgpdngavaIlkyng [symbol] itdtik | |
| | SWRNNILRTQESECACVNGSCFTIMTD | | swrnnilrtqesecacvngscftimtd | |
| | GPSNGQASYKIFRIEKGKIVKSVEMKA | | gpsngqasykifriekgkivksvemka | |
| | PNYHYEECSCYPDSSEITCVCRDNWHG | | pnyhyeecscypdsseitcvcrdnwhg | |
| | SNRPWVSFNQNLSYQIGYICSGVFGDN | | snrpwvsfnqnlsyqigyicsgvfgdn | |
| | PRPNDKTGSCGFVSSNGANGVKGFSFK | | prpndktgscgfvssngangvkgfsfk | |
| | YGNGVWIGPTKSISSRKGFEMIWDPNG | | ygngvwigrtksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSISFCGVNSDTVGWSWPDG | | ntiwtsgssisfcgvnsdtvgwswpdg | |
| | AELPFTIDK (SEQ ID NO: 14) | | aelpftidk (SEQ ID NO: 14) | |
| 164_N1- | VKLAGNSSLCPVSGWAPLSKNNAVRIG | N1 numbering: | vklagnsslcpvsgwaPLsknnavrig | 80-90% |
| [symbol] | SKGDVFVIREPFISCSPLSCRTFFLTQ | 99P/177I/196T/ | skgdvfvirepfiscsplscrtffltq | |
| [symbol] | GALLNDKHSNGTIKDRSPYRTLMSVPI | 205I, 165S, | gallndkhsngtikdrspyrtlmsvpi | |
| [symbol] | GSVPSPYNARFESIAWSASACHDGINW | 100L/408M/419V, | gSvpspynkrfesfawsasachdginw | |
| | LTIGITGPDNGAVAILKYNGIITDTIK | [symbol] | ltigiTgpdngavaIlkyngiitdtik | |
| | SWRNNILRTQESECACVNGSCFTIMTD | 103N/105A | swrnnilrtqesecacvngscftimtd | |
| | GPSDGQASYKIFRIEKGKIVKSVEMKA | | gpsdgqasykifriekgkiiksvemka | |
| | PNYHYEECSCYPDSSEITCVCRDNWHG | | pnyhyeecscypdsseitcvcrdnwhg | |
| | SNRPWVSFNQNLSYQMGYICSGVFGDN | | snrpwvsfnqnlsyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSISFCGVNSDTVGWSWPDG | | ntiwtsgssisfcgvnsdtvgwswpdg | |
| | AELPFTIDK (SEQ ID NO: 15) | | aelpftidk (SEQ ID NO: 15) | |
| 165_N1- | VKLAGNSSLCPVSGWAPLSKDNSVRIG | N1 numbering: | vklagnsslcpvsgwaPLskdnsvrig | 100% |
| [symbol] | SKGDIFVIREPFISCSPLECRTFFLTQ | 99P/177I/196T/ | skgdIfvirepfiscsplecrtffltq | |
| [symbol] | GALLNDKHSNGTIKDRSPYRTLMSVPI | 205I, 165S, | gallndkhsngtikdrspyrtlmsVpi | |
| [symbol] | GSPPSPYNARFESIAWSASACHDGINW | 100L/408M/419V, | gSPpspynArfesIawsasachdginw | |
| | LTIGITGPDSGAVAILKYNGIITDTIK | [symbol] | ltigiTgpdsgavailkyngiitdtik | |
| | SWRNNILRTQESECACVNGSCFTIMTD | 114I/166P | swrnnilrtqesecacvngscftimtd | |
| | GPSDGQASYKIFRIEKGKIVKSVEMKA | | gpsdgqasykifriekgkivksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSISFCGVNSDTVGWSWPDG | | ntiwtsgssisfcgvnsdtvgwswpdg | |
| | AELPFTIDK (SEQ ID NO: 16) | | aelpftidk (SEQ ID NO: 16) | |
| 167_N1- | VKLAGNSSLCPVSGWAPLCKDNSVRIG | N1 numbering: | vklagnsslcpvsgwaPLCkdnsvrig | 100% |
| [symbol] | SKGDVFVIREPFVSCSPLECRTFFLTQ | 99P/177I/196T/ | skgdvfvirepfVscsplecrtffltq | |
| [symbol] | GALLNDKHSNGTIKDRSPYRTLMSVP [symbol] | 205I, 165S, | gallndkhsngtikdrspyrtlmsVp [symbol] | |
| [symbol] | CSPPSPYNARVESIAWSASACHDGINW | 100L/408M/419V, | CSvpspynarVesIawsasachdginw | |
| [symbol] | LTIGITGPDSGAVAILKYNGIITDTIK | [symbol] | ltigiTgpdsgavailkyngiitdtik | |
| [symbol] | SWRNNILRTQESECACVNGSCFTIMTD | 101C/122V/164C/ | swrnnilrtqesecacvngscftimtd | |
| | GPSDGQASYKIFRIEKGKIVKSVEMKA | 174V/444V | gpsdgqasykifriekgkivksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldcIvpcfwvelirgrpee | |
| | NTIWTSGSSIVFCGVNSDTVGWSWPDG | | ntiwtsgssiVfcgvnsdtvgwswpdg | |
| | AELPFTIDK (SEQ ID NO: 17) | | aelpftidk (SEQ ID NO: 17) | |
| 174_N1- | VKLAGNSSLCPVSGWAPLSKDNSVRIG | N1 numbering: | vklagnsslcpvsgwaPLskdnsvrig | 100% |
| [symbol] | SKGDVFVIREPFVSCSPLECRQFFLTQ | 99P/177I/196T/ | skgdvfvirepfvscsplecrQffltq | |
| [symbol] | GALLNDKHSNGTIKDRSPYRTLMSVP [symbol] | 205I, 165S, | gallndkhsngtikdrspyrtlmsVp [symbol] | |
| | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | 131Q | swrnnilrtqesecacvngscftimtd | |
| | GPSDGQASYKIFRIEKGKIVKSVEMKA | | gpsdgqasykifriekgkivksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldcivpcfwvelirgrpee | |
| | NTIWTSGSSIVFCGVNSDTVGWSWPDG | | ntiwtsgssivfcgvnsdtvgwswpdg | |
| | AELPFTIDK (SEQ ID NO: 18) | | aelpftidk (SEQ ID NO: 18) | |
| 175_N1- | VKLAGNSSLCPVSGWAPLSKNNAVRIG | N1 numbering: | vklagnsslcpvsgwaPLskNnAvrig | 90-100% |
| [symbol] | SKGDVFVIREPFISCSPLECRQFFLTQ | 99P/177I/196T/ | skgdvfvirepfiscsplecrqffltq | |
| [symbol] | GALLNDKHSNGTIKDRSPYRTLMSVP [symbol] | 205I, 165S, | gallndkhsngtikdrspyrtlmsVp [symbol] | |
| [symbol] | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | [symbol] | swrnnilrtqesecacvngscftimtd | |
| | GPSDGQASYKIFRIEKGKIVKSVEMKA | 131Q | gpsdgqasykifriekgkivksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSIVFCGVNSDTVGWSWPDG | | ntiwtsgssivfcgvnsdtvgwswpdg | |
| | AELPFTIDK (SEQ ID NO: 19) | | aelpftidk (SEQ ID NO: 19) | |
| 176_N1- | VKLAGNSSLCPVSGWAPLSKDNSVRIG | N1 numbering: | vklagnsslcpvsgwaPLskdnsvrig | 100% |
| [symbol] | SKGDVFVIREPFISCSPLECRQFFLTQ | 99P/177I/196T/ | skgdvfvirepfiscsplecrQffltq | |
| [symbol] | GALLNDKHSNGTIKDRSPYRTLMSVP [symbol] | 205I, 165S, | gallndkhsngtikdrspyrtlmsVp [symbol] | |
| [symbol] | GSPPSPYNARFESIAWSASACHDGINW | 100L/408M/419V, | gsPpspynarfesiawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | 131Q, | swrnnilrtqesecacvngscftimtd | |
| | GPSDGQASYKIFRIEKGKIVKSVEMKA | 114I/166P | gpsdgqasykifriekgkivksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTVGWSWPDG | | ntiwtsgsIivfcgvnsdtvgwswpdg | |
| | AELPFTIDK (SEQ ID NO: 20) | | aelpftidk (SEQ ID NO: 20) | |
| 178_N1- | VKLAGNSSLCPVSGWAPLSKDNSVRIG | N1 numbering: | vklagnsslcpvsgwaPLskdnsvrig | 90-100% |
| [symbol] | SKGDVFVIREPFISCSPLECRQFFLTQ | 99P/177I/196T/ | skgdvfvirepfiscsplecrQffltq | |
| [symbol] | GALLNDKHSNGTIKDRSPYRTLMSVP [symbol] | 205I, 165S, | gallndkhsngtikdrspyrtlmsVp [symbol] | |
| [symbol] | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigitgpdsgavailkyngiitdtik | |
| | SWRNNILRTQESECACVNGSCFTIMTD | 131Q/442I | swrnnilrtqesecacvngscftimtd | |
| | GPSDGQASYKIFRIEKGKILKSVEMKA | | gpsdgqasykifriekgkilksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTVGWSWPDG | | ntiwtsgsIivfcgvnsdtvgwswpdg | |
| | AELPFTIDK (SEQ ID NO: 21) | | aelpftidk (SEQ ID NO: 21) | |
| 181_N1- | VKLAGNSSLCPVSGWAPLSKDNSVRIG | N1 numbering: | vklagnsslcpvsgwaPLskdnsvrig | 90-100% |
| [symbol] | SKGDVFVIREPFISCSPLECRMFFLTQ | 99P/177I/196T/ | skgdvfvirepfiscsplecrMffltq | |
| [symbol] | GALLNDKHSNGTIKDRSPYRTLMSVPI | 205I, 165S, | gallndkhsngtikdrspyrtlmsVpi | |
| [symbol] | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | 131M/442I | swrnnilrtqesecacvngscftimtd | |
| | GPSDGQASYKIFRIEKGKILKSVEMKA | | gpsdgqasykifriekgkilksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTVGWSWPDG | | ntiwtsgsIivfcgvnsdtvgwswpdg | |
| | AELPFTIDK (SEQ ID NO: 22) | | aelpftidk (SEQ ID NO: 22) | |
| 182_N1- | VKLAGNSSLCPVSGWAPLSKDNSVRIG | N1 numbering: | vklagnsslcpvsgwaPLskdnsvrig | 90-100% |
| [symbol] | SKGDVFVIREPFISCSPLECRMFFLTQ | 99P/177I/196T/ | skgdvfvirepfiscsplecrMffltq | |
| [symbol] | GALLNDKHSNGTIKDRSPYRTLMSVPI | 205I, 165S, | gallndkhsngtikdrspyrtlmsVpi | |
| [symbol] | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | 131M/442I | swrnnilrtqesecacvngscftimtd | |
| | GPSDGQASYKIFRIEKGKILKSVEMKA | | gpsdgqasykifriekgkilksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTVGWSWPDG | | ntiwtsgsIivfcgvnsdtvgwswpdg | |
| | AELPFTIDK (SEQ ID NO: 23) | | aelpftidk (SEQ ID NO: 23) | |
| 183_N1- | VKLAGNSSLCPVSGWAPLSKDNSVRIG | N1 numbering: | vklagnsslcpvsgwaPLskdnsvrig | 95-100% |
| [symbol] | SKGDVFVIREPFISCSPLECRMFFLTQ | 99P/177I/196T/ | skgdvfvirepfiscsplecrMffltq | |
| [symbol] | GALLNDKHSNGTIKDRSPYRTLMSVPL | 205I, 165S, | gallndkhsngtikdrspyrtlmsVpL | |
| [symbol] | GSVPSPYNARFESIAWSASACHDGINW | 100L/408M/419V, | gSvpspynArfesIawsasachdginw | |
| | LTIGITGPDSGAVAILKYNGIITDTIK | 161V/172A, | ltigiTgpdsgavailkyngiitdtik | |
| | SWRNNILRTQESECACVNGSCFTIMTD | [symbol] | swrnnilrtqesecacvngscftimtd | |
| | GPSDGQASYKIFRIEKGKILKSVEMKA | [symbol] | gpsdgqasykifriekgkilksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTVGWSWPDG | | ntiwtsgsIivfcgvnsdtvgwswpdg | |
| | AELPFTIDK (SEQ ID NO: 24) | | aelpftidk (SEQ ID NO: 24) | |
| 185_N1- | VKLAGNSSLCPVSGWAPLSKDNSVRIG | N1 numbering: | vklagnsslcpvsgwaPLskdnsvrig | [symbol] |
| [symbol] | SKGEIFVIREPFISCSPLECRTFFLTQ | 99P/177I/196T/ | skgEIfvirepfiscsplecrtffltq | |
| [symbol] | GALLNDKHSNGTIKDRSPYRTLMSCPI | 205I, | gallndkhsngtikdrspyrtlmscpi | |
| [symbol] | GSPPSPTNSRFESIAWSASACHDGINW | 113E/170T, | gSPpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | 114T/166F | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSIVFCGVNSDTTGWSWPDG | | ntiwtsgssivfcgvnsdtTgwswpdg | |
| | AELPFTIDK (SEQ ID NO: 25) | | aelpftidk (SEQ ID NO: 25) | |
| 194_N1- | VKLAGNSSLCPVSGWAPLSKDNSVRIG | N1 numbering: | vklagnsslcpvsgwaPLskdnsvrig | 70-80% |
| [symbol] | SKGEVFVIREPFISCSPLECRQFFLTQ | 99P/177I/196T/ | skgEvfvirepfiscsplecrQffltq | |
| [symbol] | GALLNDKHSNGTIKDRSPYRTLMSCPI | 205I, | gallndkhsngtikdrspyrtlmscpi | |
| [symbol] | GSVPSPTNSRFESIAWSASACHDGINW | 113E/170T, | gSvpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, [symbol] | gpsdgqasykifriekgkilksvemka | |
| | PNYHYEECSCYPDSSEITCVCRDNWHG | | pnyhyeecscypdsseitcvcrdnwhg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSIVFCGVNSDTTGWSWPDG | | ntiwtsgssivfcgvnsdtTgwswpdg | |
| | AELPFTIDK (SEQ ID NO: 26) | | aelpftidk (SEQ ID NO: 26) | |
| 195_N1- | VKLAGNSSLCPVSGWAPLSKDNAVRIG | N1 numbering: | vklagnsslcpvsgwaPLskdnAvrig | 50-60% |
| [symbol] | SKGEVFVIREPFISCSPLECRQFFLTQ | 99P/177I/196T/ | skgEvfvirepfiscsplecrQffltq | |
| [symbol] | GALLNDKHSNGTIKDRSPYRTLMSCPI | 205I, | gallndkhsngtikdrspyrtlmscpi | |
| [symbol] | GSVPSPTNSRFESIAWSASACHDGINW | 113E/170T, | gSvpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | [symbol] | pnyhyeecscypdsseitcvcrdnwhg | |
| | SNRPWVSFNQNLEYQMGYICSGVFGDN | [symbol] | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSIVFCGVNSDTTGWSWPDG | | ntiwtsgssivfcgvnsdtTgwswpdg | |
| | AELPFTIDK (SEQ ID NO: 27) | | aelpftidk (SEQ ID NO: 27) | |
| 196_N1- | VKLAGNSSLCPVSGWAPLSKDNSVRIG | N1 numbering: | vklagnsslcpvsgwaPLskdnsvrig | 40% |
| [symbol] | SKGEIFVIREPFISCSPLECRQFFLTQ | 99P/177I/196T/ | skgEIfvirepfiscsplecrQffltq | |
| [symbol] | GALLNDKHSNGTIKDRSPYRTLMSCPI | 205I, | gallndkhsngtikdrspyrtlmscpi | |
| [symbol] | GSPPSPTNSRFESIAWSASACHDGINW | 113E/170T, | gSPpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | [symbol] | pnyhyeecscypdsseitcvcrdnwhg | |
| | SNRPWVSFNQNLEYQMGYICSGVFGDN | T131Q | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSSIVFCGVNSDTTGWSWPDG | | ntiwtsgssivfcgvnsdtTgwswpdg | |
| | AELPFTIDK (SEQ ID NO: 28) | | aelpftidk (SEQ ID NO: 28) | |
| 198_N1- | VKLAGNSSLCPVSGWAPLSKDNSVRIG | N1 numbering: | vklagnsslcpvsgwaPLskdnsvrig | 20-30% |
| [symbol] | SKGEVFVIREPFISCSPLECRQFFLTQ | 99P/177I/196T/ | skgEvfvirepfiscsplecrQffltq | |
| [symbol] | GALLNDKHSNGTIKDRSPYRTLMSCPI | 205I, | gallndkhsngtikdrspyrtlmscpi | |
| [symbol] | GSVPSPTNSRFESIAWSASACHDGINW | 113E/170T, | gSvpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | [symbol] | pnyhyeecscypdsseitcvcrdnwhg | |
| | SNRPWVSFNQNLEYQMGYICSGVFGDN | T131Q/4421I | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTTGWSWPDG | | ntiwtsgsIivfcgvnsdtTgwswpdg | |
| | AELPFTIDK (SEQ ID NO: 29) | | aelpftidk (SEQ ID NO: 29) | |
| 201_N1- | VKLAGNSSLCPVSGWAPLSKDNSVRIG | N1 numbering: | vklagnsslcpvsgwaPLskdnsvrig | 50% |
| [symbol] | SKGEVFVIREPFISCSPLECRMFFLTQ | 99P/177I/196T/ | skgEvfvirepfiscsplecrMffltq | |
| [symbol] | GALLNDKHSNGTIKDRSPYRTLMSCPI | 205I, | gallndkhsngtikdrspyrtlmscpi | |
| [symbol] | GSVPSPTNSRFESIAWSASACHDGINW | 113E/170T, | gSvpspTnsrfesIawsasachdginw | |
| | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | [symbol] | pnyhyeecscypdsseitcvcrdnwhg | |
| | SNRPWVSFNQNLEYQMGYICSGVFGDN | T131Q/4421I | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTTGWSWPDG | | ntiwtsgsIivfcgvnsdtTgwswpdg | |
| | AELPFTIDK (SEQ ID NO: 30) | | aelpftidk (SEQ ID NO: 30) | |
| 202_N1- | VKLAGNSSLCPVSGWAPLSKNNAVRIG | N1 numbering: | vklagnsslcpvsgwaPLskNnAvrig | 20% |
| [symbol] | SKGEVFVIREPFISCSPLECRMFFLTQ | 99P/177I/196T/ | skgEvfvirepfiscsplecrMffltq | |
| [symbol] | GALLNDKHSNGTIKDRSPYRTLMSCPI | 205I, | gallndkhsngtikdrspyrtlmscpi | |
| [symbol] | GSVPSPTNSRFESIAWSASACHDGINW | 113E/170T, | gSvpspTnsrfesIawsasachdginw | |
| [symbol] | LTIGITGPDSGAVAILKYNGIITDTIK | 165S, | ltigiTgpdsgavailkyngiitdtik | |
| | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | 103N/105A, | pnyhyeecscypdsseitcvcrdnwhg | |
| | SNRPWVSFNQNLEYQMGYICSGVFGDN | T131M/4421I | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIVFCGVNSDTTGWSWPDG | | ntiwtsgsIivfcgvnsdtTgwswpdg | |
| | AELPFTIDK (SEQ ID NO: 31) | | aelpftidk (SEQ ID NO: 31) | |
| 203_N1- | VKLAGNSSLCPVSGWAPLAK [symbol] NSVRIG | N1 numbering: | vklagnsslcpvsgwaPLAk [symbol] nsvrig | 90% |
| [symbol] | SKGEVFVIREPFISCSPLECRMFFLTQ | 99P/177I/196T/ | skgEvfvirepfiscsplecrMffltq | |
| [symbol] | GALLNDKHSNGTIKDRSPYRTLMSCPL | 205I, | gallndkhsngtikdrspyrtlmscpL | |
| [symbol] | GSVPSPTNSRFESIAWSASACHDGINW | 113E/170T, | gSvpspTnsrfesIawsasachdginw | |
| [symbol] | LTIGITGPDSGAVAILKYNGIITDTIK | 165S, | ltigiTgpdsgavailkyngiitdtik | |
| [symbol] | SWRNNILRTQESECACVNGSCFTIMTD | 100L/408M/419V, | swrnnilrtqesecacvngscftimtd | |
| | GPSDGQASYKIFRIEKGKILKSVEMKA | 453T, | gpsdgqasykifriekgkilksvemka | |
| | PNYHYEECSCYPDSSEITCVCRDNWHG | 101A/163L/T151S/ | pnyhyeecscypdsseitcvcrdnwhg | |
| | SNRPWVSFNQNLEYQMGYICSGVFGDN | 4421I/444A | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGPTKSISSRKGFEMIWDPNG | | ygngvwigptksissrkgfemiwdpng | |
| | WTGTDNKFSIKQDIVGINEWSGYSGSF | | wtgtdnkfsikqdivginewsgysgsf | |
| | VMHPELTGLDCIVPCFWVELIRGRPEE | | vMhpeltgldciVpcfwvelirgrpee | |
| | NTIWTSGSIIAFCGVNSDTTGWSWPDG | | ntiwtsgsIiAfcgvnsdtTgwswpdg | |
| | AELPFTIDK (SEQ ID NO: 32) | | aelpftidk (SEQ ID NO: 32) | |
| 249_N2- | EYRNWSKPQSNITGFAPFSKDNSIRLS | N2 numbering: | eyrnwskpqsnitgfapfskdnsirls | 50% |
| [symbol] | AGGDIWVTREPYVSCSPDKCYQFALGQ | 165Q | aggdiwvtrepyvscspdkcyqfalgq | closed |
| [symbol] | GTTLNNVRSNDTVHDRTPYRTLIMNEL | (mutations | gttlnnvrsndtvhdrtpyrtlimnel | [symbol] |
| | GQPPHLGTKQVCIAWSSSSCHDGKAWL | promote open | gQpphlgtkqvciawsssschdgkawl | open) |
| | HVCVTGDDKNATASFIYNGPLVDSIVS | state of NA) | hvcvtgddknatasfiyngplvdsivs | |
| | WSKEILRTQESECVCINGTCTVVMTDG | | wskeilrtqesecvcingtctvvmtdg | |
| | SASCKADTKILFIEEGKIVHTSTLSGS | | sasckadtkilfieegkivhtstlsgs | |
| | AQHVEECSCYPRYLGVPGYCRDNWKGS | | aqhveecscyprylgvpgycrdnwkgs | |
| | NRPIVDINIKDYSIVSSYVCSCLVGDT | | nrpivdinikdysivssyvcsclvgdt | |
| | PRKNDSSSSSHCLDPNNEEGGHGVKGW | | prkndssssshcldpnneegghgvkgw | |
| | AFDSGNDVWNGRTISEKLRSGYETFKV | | afdsgndvwngrtiseklrsgyetfkv | |
| | IEGWSNPNSKLQINRQVIVDRGNRSGY | | iegwsnpnsklqinrqvivdrgnrsgy | |
| | SGTPSVSGKSCINRCFYVELIPSRKEE | | sgtpsvsgkscinrcfyvelipsrkee | |
| | TEVLWTSNSIVVFCGTSGTYGTGSWPD | | tevlwtsnsivvfcgtsgtygtgswpd | |
| | GADINLMP [symbol] (SEQ ID NO: 33) | | gadinlmp [symbol] (SEQ ID NO: 33) | |
| 255_N2- | EYRNWSKPQSNITGFAPFSKDNSIRLS | N2 numbering: | eyrnwskpqsnitgfapfskdnsirls | 5% |
| [symbol] | AGGDIWVTREPYVSCSPDKCYQFALGQ | 165Q, 176I, | aggdiwvtrepyvscspdkcyqfalgq | closed |
| [symbol] | GTTLNNVRSNDTVHDRTPYRTLIMNEL | [symbol] | gttlnnvrsndtvhdrtpyrtlimnel | (50% |
| [symbol] | GQPPHLGTKQVCVAWSSSSCHDGKAWL | (mutations | gQpphlgtkqvcVawsssschdgkawl | open) |
| | HVCVSGDDKNATASFIYNGPLVDSIVS | promote open | hvcvSgddknatasfiyngplvdsivs | |
| | WSKEILRTQESECVCINGTCTVVMTDG | state of NA) | wskeilrtqesecvcingtctvvmtdg | |
| | SASCKADTKILFIEEGKIVHTSTLSGS | | sasckadtkilfieegkivhtstlsgs | |
| | AQHVEECSCYPRYLGVPGYCRDNWKGS | | aqhveecscyprylgvpgycrdnwkgs | |
| | NRPIVDINIKDYSIVSSYVCSCLVGDT | | nrpivdinikdysivssyvcsclvgdt | |
| | PRKNDSSSSSHCLDPNNEEGGHGVKGW | | prkndssssshcldpnneegghgvkgw | |
| | AFDSGNDVWNGRTISEKLRSGYETFKV | | afdsgndvwngrtiseklrsgyetfkv | |
| | IEGWSNPNSKLQINRQVIVDRGNRSGY | | iegwsnpnsklqinrqvivdrgnrsgy | |
| | SGTFSVSGKSCINRCFYVELIPSRKEE | | sgtfsvsgkscinrcfyvelipsrkee | |
| | TEVLWTSNSIVVFCGTSGTYGTGSWPD | | tevlwtsnsivvfcgtsgtygtgswpd | |
| | GADINLMP [symbol] (SEQ ID NO: 34) | | gadinlmp [symbol] (SEQ ID NO: 34) | |
| 282_N8- | HFMNNTEALCDAKGFAPFSKDNSIRIG | N8 numbering: | hfmnntealcdakgfapfskdnsirig | 60% |
| [symbol] | SRGHVFVIREPYVSCSPTECRTFFLTQ | 163S | srghvfvirepyvscsptecrtffltq | |
| [symbol] | GSLLNDKHSNGTVKDRSPYRTLMSVEI | | gsllndkhsngtvkdrspyrtlmsvei | |
| [symbol] | GSSPNVYQARFEAVAWSATACHDGKKW | | gSspnvyqarfeavawsatachdgkkw | |
| | MTIGVTGPDAKAVAVVHYGGIPTDVIN | | mtigvtgpdakavavvhyggiptdvin | |
| | SWAGDILRTQESSCFCIQGECFWVNTD | | swagdilrtqesscfciqgecfwvntd | |
| | GPAGRQAQYNAFNAKQGKIBGQGEISF | | gpagrqaqyrsfkakqgkivgqaeisf | |
| | NGGHIRKCSCYPNEGEVECVCKDNWTG | | ngghirkcscypnegevecvckdnwtg | |
| | TNRPVLVISPDLSYRYGYLCAGLPSDT | | tnrpvlvispdlsyrygylcaglpsdt | |
| | PRGEDSQFTGSCTSPMGNGQYGWKGFG | | prgedsqftgsctspmgngqygwkgfg | |
| | FRQGNDVWNGRTISKTSKDGFEILKVR | | frqgndvwngrtisktskdgfeilkvr | |
| | NGWVQNSKEQIKEQVVVDNLNWSGYSG | | ngwvqnskeqikeqvvvdnlnwsgysg | |
| | SFRKPAEKTKRBCKVPCFWVEMIRGNP | | sfrkpaektkrbckvpcfwvemirgnp | |
| | EEKTIWTSSSSIVMCGVDHAIADWSWH | | eektiwtssssivmcgvdhaiadwswh | |
| | DGAILPFDIDKM (SEQ ID NO: 35) | | dgailpfdidkm (SEQ ID NO: 35) | |
| 285_N8- | HFMNNTEALCDAKGFAPFSKDNSIRIG | N8 numbering: | hfmnntealcdakgfapfskdnsirig | 100% |
| [symbol] | SRGHVFVIREPFVSCSPTECRTFFLTQ | [symbol] , 163S | srghvfvirepfvscsptecrtffltq | |
| [symbol] | GSLLNDKHSNGTVKDRSPYRTLMSVPI | | gsllndkhsngtvkdrspyrtlmsvPi | |
| [symbol] | GSSPNVYQARFEAVAWSATACHDGKKW | | gSspnvyqarfeavawsatachdgkkw | |
| [symbol] | MTIGVTGPDAKAVAVVHYGGIPTDVIN | | mtigvtgpdakavavvhyggiptdvin | |
| | SWAGDILRTQESSCFCIQGECFWVMTD | | swagdilrtqesscfciqgecfwvmtd | |
| | GPAGRQAQYNAFNAKQGKIBGQGEISF | | gpagrqaqyrsfkakqgkivgqaeisf | |
| | NGGHIRKCSCYPNEGEVECVCKDNWTG | | ngghirkcscypnegevecvckdnwtg | |
| | TNRPVLVISPDLSYRYGYLCAGLPSDT | | tnrpvlvispdlsyrygylcaglpsdt | |
| | PRGEDSQFTGSCTSPMGNGQYGWKGFG | | prgedsqftgsctspmgngqygwkgfg | |
| | FRQGNDVWNGRTISKTSKDGFEILKVR | | frqgndvwngrtisktskdgfeilkvr | |
| | NGWVQNSKEQIKEQVVVDNLNWSGYSG | | ngwvqnskeqikeqvvvdnlnwsgysg | |
| | SFRKPAEKTKRBCKVPCFWVEMIRGNP | | sfrkpaektkrbckvpcfwvemirgnp | |
| | EEKTIWTSSSSIVMCGVDHAIADWSWH | | eektiwtssssivmcgvdhaiadwswh | |
| | DGAILPFDIDKM (SEQ ID NO: 36) | | dgailpfdidkm (SEQ ID NO: 36) | |
| 288_N8- | HFMNNTEALCDAKGFAPFSKDNSIRIG | N8 numbering: | hfmnntealcdakgfapfskdnsirig | |
| [symbol] | SRGHVFVIREPFVSCSPTECRTFFLTQ | 160P, 163S | srghvfvirepfvscsptecrtffltq | |
| [symbol] | GSLLNDKHSNGTVKDRSPYRTLMSVPI | 175I, | gsllndkhsngtvkdrspyrtlmsvPi | |
| [symbol] | GSSPNVYQARFEAVAWSATACHDGKKW | | gSspnvyqarfeavawsatachdgkkw | |
| [symbol] | MTIGVTGPDAKAVAVVHYGGIPTDVIN | | mtigvtgpdakavavvhyggiptdvin | |
| [symbol] | SWAGDILRTQESSCFCIQGECFWVMTD | | swagdilrtqesscfciqgecfwvmtd | |
| | GPAGRQAQYNAFNAKQGKIBGQGEISF | | gpagrqaqyrsfkakqgkivgqaeisf | |
| | NGGHIEECSCYPNEGEVECVCKDNWTG | | ngghieecscypnegevecvckdnwtg | |
| | TNRPVLVISPDLSYRYGYLCAGLPSDT | | tnrpvlvispdlsyrygylcaglpsdt | |
| | PRGEDSQFTGSCTSPMGNGQYGVKGFG | | prgedsqftgsctspmgngqygvkgfg | |
| | FRQGNDVWNGRTISKTSKDGFEILKVR | | frqgndvwngrtisktskdgfeilkvr | |
| | NGWVQNSKEQIKEQVVVDNLNWSGYSG | | ngwvqnskeqikeqvvvdnlnwsgysg | |
| | SFTLPAEKTKRBCKVPCFWVEMIRGNP | | sftlpaektkrbckvpcfwvemirgnp | |
| | EEKTIWTSSSSIVMCGVDHAIADWSWH | | eektiwtssssivmcgvdhaiadwswh | |
| | DGAILPFDIDKM (SEQ ID NO: 37) | | dgailpfdidkm (SEQ ID NO: 37) | |
| 289_N8- | HFMNNTEALCDAKGFAPFSKDNGIRIG | N8 numbering: | hfmnntealcdakgfapfskdngirig | 70% |
| [symbol] | SRGHVFVIREPFVSCSPTECRTFFLTQ | 160P, 163S | srghvfvirepfvscsptecrtffltq | |
| [symbol] | GSLLNDKHSNGTVKDRSPYRTLMSVPI | 175I, [symbol] | gsllndkhsngtvkdrspyrtlmsvPi | |
| [symbol] | GSSPNVYQARFEAVAWSATACHDGKKW | | gSspnvyqarfeavawsatachdgkkw | |
| [symbol] | MTIGVTGPDAKAVAVVHYGGIPTDVIN | | mtigvtgpdakavavvhyggiptdvin | |
| [symbol] | SWAGDILRTQESSCFCIQGECFWVMTD | | swagdilrtqesscfciqgecfwvmtd | |
| [symbol] | GPAGRQAQYNAFNAKQGKIBGQGEISF | | gpagrqaqyrsfkakqgkivgqaeisf | |
| | NGGHIEECSCYPNEGEVECVCKDNWTG | | ngghieecscypnegevecvckdnwtg | |
| | TNRPVLVISPDLSYRYGYLCAGLPSDT | | tnrpvlvispdlsyrygylcaglpsdt | |
| | PRGEDSQFTGSCTSPMGNGQYGVKGFG | | prgedsqftgsctspmgngqygvkgfg | |
| | FRQGNDVWNGRTISKTSKDGFEILKVR | | frqgndvwngrtisktskdgfeilkvr | |
| | NGWVQNSKEQIKEQVVVDNLNWSGYSG | | ngwvqnskeqikeqvvvdnlnwsgysg | |
| | SFTLPAEKTKRBCKVPCFWVEMIRGNP | | sftlpaektkrbckvpcfwvemirgnp | |
| | EEKTIWTSSSSIVMCGVDHAIADWSWH | | eektiwtssssivmcgvdhaiadwswh | |
| | DGAILPFDIDKM (SEQ ID NO: 38) | | dgailpfdidkm (SEQ ID NO: 38) | |
| [symbol] | VILTGNSSICPISGWAPLAKDNSIRIG | N1 numbering: | viltgnssIcpisgwaPLAkdnSirig | 20-30% |
| [symbol] | SKGDVFVIPEPRISCSMLECRMFFLTQ | 99P/177I/196T/ | skgdvfvipepriscsmlecrMffltq | |
| [symbol] | GALLNDKHSNGTVKSPSPYFRLMSVPL | 205L, 165S, | gallndkhsngtvkspspytrlmsVpL | |
| [symbol] | GSVPSPYNARFESIAWSASACHDGMGW | [symbol] | gSVpspynArfesIawsasachdgmgw | |
| [symbol] | LTIGITGPDNGAVAILKYNGIITDTIK | [symbol] | ltigiTgpdngavailkyngiitdtik | |
| [symbol] | SWPRNILRTQESECACVNGSCFTIMTD | 101A/131M/163L/ | swprnilrtqesecacvngscftimtd | |
| [symbol] | GPSNGQASYKILKIEKGKVRKSIELNA | 442I/444A, | gpsngqasykilkiekgkvrksielna | |
| [symbol] | PNYHYEECSCYPDTGKVMCVCRDNWHG | 165S | pnyhyeecscypdtgkvmcvcrdnwhg | |
| | SNRPWVSFDQNLDYQIGYICSGVFGDN | | snrpwvsfdqnldyqigyicsgvfgdn | |
| | PRPNDGTGSCGPVSSWGAMGIFGFSFR | | prpndgtgscgpvsswgamgifgfsfr | |
| | YDNGVWDGRTKSTSSRSGFEMIWDPNG | | ydngvwdgrtkstssrsgfemiwdpng | |
| | WTETDSSFSVRQDIVAITDWSGYSGSF | | wtetdssfsvrqdivaitdwsgysgsf | |
| | VMHPKLTGLDCMVPCFWVSLIRGQPKE | | vMhpkltgldcmVpcfwvslirgqpke | |
| | NTIWTSGSIIAFCGVNSDTVGWSWPDG | | ntiwtsgsIiAfcgvnsdtvgwswpdg | |
| | AELPFSLDK (SEQ ID NO: 39) | | aelpfsldk (SEQ ID NO: 39) | |
| 354_N1- | VKLAGNSSLCPINGWAPLSKDNSVRIG | N1 numbering: | vklagnsslcpingwaPLskdnsVrig | [symbol] |
| [symbol] | SKGDVFVIREPFISCSMLECRQFFLTQ | 99P/177I/196T/ | skgdvfvirepfiscshlecrQffltq | |
| [symbol] | GALLNDKHSNGTVKSPSPYFRLMSVPI | 205L, 165S, | gallndkhsngtvkspspyfrlmsVpI | |
| [symbol] | GSVPSPYNARFESIAWSASACHDGTSW | [symbol] | gSVpspynArfesIawsasachdgtsw | |
| [symbol] | LTIGITGPDNGAVAILKYNGIITDTIK | [symbol] | ltigiTgpdngavailkyngiitdtik | |
| | SWFNNILRTQESECACVNGSCFTVMTD | 131Q, [symbol] | swfnnilrtqesecacvngscftvmtd | |
| | GPSNGQASYKIFMEKGKVVRKSVELDA | 163I, 166V | gpsngqasykifmekgkvvrksvelda | |
| | PNYHIEECSCYPNAGEITCVCRDNWHG | | pnyhieecscypnageitcvcrdnwhg | |
| | SNRPWVSFDQNLDYQIGYICSGVFGDN | | snrpwvsfdqnldyqigyicsgvfgdn | |
| | PRPNDGTGSCSGPVSSGAYGVKGFSFK | | prpndgtgscsgpvssgaygvkgfsfk | |
| | YGNGVWIGRTKSTNSRSGFEMIWDPNG | | ygngvwigrtkstnsrsgfemiwdpng | |
| | WTETDSSFSVKQDIVSITDWSGYSGSF | | wtetdssfsvkqdivsitdwsgysgsf | |
| | VMHPKLTGLDCMVPCFWVSLIRGQPKE | | vMhpkltgldciVpcfwvslirgrpke | |
| | STIWTSGSSISFCGVNSDTVGWSWPDG | | stiwtsgssisfcgvnsdtvgwswpdg | |
| | AELPFTLDK (SEQ ID NO: 40) | | aelpftldk (SEQ ID NO: 40) | |
| 356_N1- | VKLAGNSSLCPINGWAPLSKDNSVRIG | N1 numbering: | vklagnsslcpingwaPLskdnsVrig | [symbol] |
| [symbol] | SKGDIFVIREPFISCSMLECRQFFLTQ | 99P/177I/196T/ | skgdIfvirepfiscshlecrQffltq | |
| [symbol] | GALLNDKHSNGTVKSPSPYFRLMSVPI | 205L, 165S, | gallndkhsngtvkspspyfrlmsVpI | |
| [symbol] | GSPPSPYNARFESIAWSASACHDGTSW | [symbol] | gSPpspynArfesIawsasachdgtsw | |
| [symbol] | LTIGITGPDNGAVAILKYNGIITDTIK | [symbol] | ltigiTgpdngavailkyngiitdtik | |
| [symbol] | SWFNNILRTQESECACVNGSCFTVMTD | 131Q, [symbol] | swfnnilrtqesecacvngscftvmtd | |
| | GPSNGQASYKIFMEKGKVVRKSVELDA | 163I, | gpsngqasykifmekgkvvrksvelda | |
| | PNYHYEECSCYPNAGEITCVCRDNWHG | 114I/ [symbol] | pnyhyeecscypnageitcvcrdnwhg | |
| | SNRPWVSFDQNLDYQIGYICSGVFGDN | | snrpwvsfdqnldyqigyicsgvfgdn | |
| | PRPNDGTGSCSGPVSSGAYGVKGFSFK | | prpndgtgscsgpvssgaygvkgfsfk | |
| | YGNGVWIGRTKSTNSRSGFEMIWDPNG | | ygngvwigrtkstnsrsgfemiwdpng | |
| | WTETDSSFSVKQDIVSITDWSGYSGSF | | wtetdssfsvkqdivsitdwsgysgsf | |
| | VMHPKLTGLDCMVPCFWVSLIRGQPKE | | vMhpkltgldciVpcfwvslirgrpke | |
| | STIWTSGSSISFCGVNSDTVGWSWPDG | | stiwtsgssisfcgvnsdtvgwswpdg | |
| | AELPFTLDK (SEQ ID NO: 41) | | aelpftldk (SEQ ID NO: 41) | |
| 357_N1- | VKLAGNSSLCPINGWAPLSKDNSVRIG | N1 numbering: | vklagnsslcpingwaPLskdnsVrig | 70% |
| [symbol] | SKGDIFVIREPFISCSPLECRQFFLTQ | 99P/177I/196T/ | skgdIfvirepfiscsPlecrQffltq | |
| [symbol] | GALLNDKHSNGTVKSPSPYFRLMSVPI | 205L, 165S, | gallndkhsngtvkspspyfrlmsVpI | |
| [symbol] | GSPPSPYNARFESIAWSASACHDGTSW | 100L/408M/419V, | gSPpspynArfesIawsasachdgtsw | |
| [symbol] | LTIGITGPDNGAVAILKYNGIITDTIK | [symbol] | ltigiTgpdngavaIlkyngiitdtik | |
| [symbol] | SWFNNILRTQESECACVNGSCFTVMTD | 131Q, 106V | swfnnilrtqesecacvngscftvmtd | |
| [symbol] | GPSNGQASYKIFKIEKGKIVKSVEMDA | 163I, | gpsngqasykifkIekgkivksveMda | |
| | PNYHYEECSCYPNAGEITCVCRDNWHG | 114I/166P | pnyhyeecscypnageitcvcrdnwhg | |
| | SNRPWVSFDQNLDYQIGYICSGVFGDN | [symbol] | snrpwvsfdqnldyqigyicsgvfgdn | |
| | PRPNDGTGSCSGPVSSGAYGVKGFSFK | 257I, 262I | prpndgtgscsgpvssgaygvkgfsfk | |
| | YGNGVWIGRTKSTNSRSGFEMIWDPNG | 268M | ygngvwigrtkstnsrsgfemiwdpng | |
| | WTETDSSFSVKQDIVSITDWSGYSGSF | | wtetdssfsvkqdivsitdwsgysgsf | |
| | VMHPKLTGLDCMVPCFWVSLIRGQPKE | | vMhpkltgldciVpcfwvslirgrpke | |
| | STIWTSGSSISFCGVNSDTVGWSWPDG | | stiwtsgssisfcgvnsdtvgwswpdg | |
| | AELPFTLDK (SEQ ID NO: 42) | | aelpftldk (SEQ ID NO: 42) | |
| 366_N1- | VILTGNSSLCPIRGWAPLSKDNSVRIG | N1 numbering: | viltgnsslcpirgwaPLskdnSVrig | [symbol] |
| [symbol] | SKGDVFVIREPFISCSPLECRTFFLTQ | 99P/177I/196T/ | skgdvfvirepfiscsPlecrtffltq | |
| [symbol] | GALLNDKSSRGTFKDRSPYRTLMSVPI | 205L, 165S, | gallndkssrgtfkdrspyrTlmsVpI | |
| [symbol] | GSVPSPYNARFESIAWSASACHDGMGW | 100L/408M/419V, | gSVpspynArfesIawsasachdgmgw | |
| [symbol] | LTIGITGPDNGAVAILKYNGIITETIK | [symbol] | ltigiTgpdngavaIlkynGiitetik | |
| [symbol] | SWRKNILRTQESECTCVNGSCFTIMTD | 131Q, 106V | swrknilrtqesectcvngscftimtd | |
| [symbol] | GPSDGLASYKIFKIEKGKVTKSIELNA | 163I, | gpsdglasykifkiekgkvtksielna | |
| | PNSHYEECSCYPDTGKVMCVCRDNWHG | 114I/166P | pnshyeecscypdtgkvmcvcrdnwhg | |
| | SNRPWVSFDQNLDYQIGYICSGVFGDN | [symbol] | snrpwvsfdqnldyqigyicsgvfgdn | |
| | PRPKDGTGSCGPVSADGANGVKGFSYK | 257I, 262I | prpkdgtgscgpvsadgangvkgfsyk | |
| | YGNGVWIGRTKSTNSRSGFEMIWDPNG | 268M | ygngvwigrtkstnsrsgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGLPEE | | vMhpeltgldcmVpcfwvelirglpee | |
| | DAIWTSGSIISFCGVNSDTVDWSWPDG | | daiwtsgsiisfcgvnsdtvdwswpdg | |
| | AELPFTLDK (SEQ ID NO: 43) | | aelpftldk (SEQ ID NO: 43) | |
| 367_N1- | VILTGNSSLCPIRGWAPLSKDNSVRIG | N1 numbering: | viltgnsslcpirgwaPLskdnSVrig | |
| [symbol] | SKGDVFVIREPFISCSPLECRTFFLTQ | 99P/177I/196T/ | skgdvfvirepfiscsPlecrQffltq | |
| [symbol] | GALLNDKSSRGTFKDRSPYRTLMSVPI | 205L, 165S, | gallndkssrgtfkdrspyrTlmsVpI | |
| [symbol] | GSVPSPYNARFESIAWSASACHDGMGW | 100L/408M/419V, | gSVpspynArfesIawsasachdgmgw | |
| [symbol] | LTIGITGPDNGAVAILKYNGIITETIK | [symbol] | ltigiTgpdngavaIlkynGiitetik | |
| [symbol] | SWRKNILRTQESECTCVNGSCFTIMTD | 131Q, [symbol] | swrknilrtqesectcvngscftimtd | |
| [symbol] | GPSDGLASYKIFKIEKGKVTKSIELNA | [symbol] | gpsdglasykifkiekgkvtksielna | |
| | PNSHYEECSCYPDTGKVMCVCRDNWHG | [symbol] | pnshyeecscypdtgkvmcvcrdnwhg | |
| | SNRPWVSFDQNLDYQIGYICSGVFGDN | [symbol] | snrpwvsfdqnldyqigyicsgvfgdn | |
| | PRPKDGTGSCGPVSADGANGVKGFSYK | | prpkdgtgscgpvsadgangvkgfsyk | |
| | YGNGVWIGRTKSTNSRSGFEMIWDPNG | | ygngvwigrtkstnsrsgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGLPEE | | vMhpeltgldcmVpcfwvelirglpee | |
| | DAIWTSGSIISFCGVNSDTVDWSWPDG | | daiwtsgsiisfcgvnsdtvdwswpdg | |
| | AELPFTLDK (SEQ ID NO: 44) | | aelpftldk (SEQ ID NO: 44) | |
| 369_N1- | VILTGNSSLCPIRGWAPLSKDNSVRIG | N1 numbering: | viltgnsslcpirgwaPLskdnSVrig | 70% |
| [symbol] | SKGDVFVIREPFISCSPLECRTFFLTQ | 99P/177I/196T/ | skgdvfvirepfiscsPlecrQffltq | |
| [symbol] | GALLNDKSSRGTFKDRSPYRTLMSVPI | 205L, 165S, | gallndkssrgtfkdrspyrTlmsVpI | |
| [symbol] | GSVPSPYNARFESIAWSASACHDGMGW | 100L/408M/419V, | gSVpspynArfesIawsasachdgmgw | |
| [symbol] | LTIGITGPDNGAVAILKYNGIITETIK | [symbol] | ltigiTgpdngavaIlkynGiitetik | |
| [symbol] | SWRKNILRTQESECTCVNGSCFTIMTD | 131Q, [symbol] | swrknilrtqesectcvngscftimtd | |
| [symbol] | GPSDGLASYKIFKIEKGKVTKSIELNA | [symbol] | gpsdglasykifkiekgkvtksielna | |
| [symbol] | PNSHYEECSCYPDTGKVMCVCRDNWHG | [symbol] | pnshyeecscypdtgkvmcvcrdnwhg | |
| | SNRPWVSFDQNLDYQIGYICSGVFGDN | [symbol] | snrpwvsfdqnldyqigyicsgvfgdn | |
| | PRPKDGTGSCGPVSADGANGVKGFSYK | | prpkdgtgscgpvsadgangvkgfsyk | |
| | YGNGVWIGRTKSTNSRSGFEMIWDPNG | | ygngvwigrtkstnsrsgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGLPEE | | vMhpeltgldcmVpcfwvelirglpee | |
| | DAIWTSGSIISFCGVNSDTVDWSWPDG | | daiwtsgsiisfcgvnsdtvdwswpdg | |
| | AELPFTLDK (SEQ ID NO: 45) | | aelpftldk (SEQ ID NO: 45) | |
| 371_N1- | VILTGNSSLCPIRGWAPLSKDNSVRIG | N1 numbering: | viltgnsslcpirgwaPLskdnSVrig | 80% |
| [symbol] | SKGDVFVIREPFISCSPLECRTFFLTQ | 99P/177I/196T/ | skgdvfvirepfiscsPlecrQffltq | |
| [symbol] | GALLNDKSSRGTFKDRSPYRTLMSVPI | 205L, 165S, | gallndkssrgtfkdrspyrTlmsVpI | |
| [symbol] | GSVPSPYNARFESIAWSASACHDGMGW | 100L/408M/419V, | gSVpspynArfesIawsasachdgmgw | |
| [symbol] | LTIGITGPDNGAVAILKYNGIITETIK | 161V/172A, | ltigiTgpdngavaIlkynGiitetik | |
| [symbol] | SWRKNILRTQESECTCVNGSCFTIMTD | [symbol] /166P, | swrknilrtqesectcvngscftimtd | |
| [symbol] | GPSDGLASYKIFKIEKGKVTKSIELNA | [symbol] | gpsdglasykifkiekgkvtksielna | |
| [symbol] | PNSHYEECSCYPDTGKVMCVCRDNWHG | 106V, [symbol] | pnshyeecscypdtgkvmcvcrdnwhg | |
| [symbol] | SNRPWVSFDQNLDYQIGYICSGVFGDN | 163I, 210G, | snrpwvsfdqnldyqigyicsgvfgdn | |
| | PRPKDGTGSCGPVSADGANGVKGFSYK | 442S | prpkdgtgscgpvsadgangvkgfsyk | |
| | YGNGVWIGRTKSTNSRSGFEMIWDPNG | | ygngvwigrtkstnsrsgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGLPEE | | vMhpeltgldcmVpcfwvelirglpee | |
| | DAIWTSGSIISFCGVNSDTVDWSWPDG | | daiwtsgsiisfcgvnsdtvdwswpdg | |
| | AELPFTLDK (SEQ ID NO: 46) | | aelpftldk (SEQ ID NO: 46) | |
| 399_N1- | VKLAGNSSLCPVSGWAPLSKDNSVRIG | N1 numbering: | vklagnsslcpvsgwaPLskdnsvrig | |
| [symbol] | SKGDVPVIREPFISCSPLECRTFFLTQ | 99P/177I/196T/ | skgdvpvirepfiscsplecrtffltq | |
| [symbol] | GALLNDKHSNGTIKDRSPYRTLMSVPI | 165S, | gallndkhsngtikdrspyrtlmsVpi | |
| [symbol] | GSVRSRYNARFESIAWSASACHDGINW | 100L/408M/419V, | gSvrsrynArfesIawsasachdginw | |
| | LTIGITGPDNFAVAVLKYNGIITDTIK | 161V/172A, | ltigiTgpdnfavavlkyngiitdtik | |
| | SWRNMILRTQESECACVNGSCFTVMTG | | swrnsilrtqesecacvngscftvmtg | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 47) | | aelpftldk (SEQ ID NO: 47) | |
| 400_N1- | VKLAGNSSLCPVSGWAPLSKDNSVRIG | N1 numbering: | vklagnsslcpvsgwaPLskdnsvrig | |
| [symbol] | SKGDVPVIREPFISCSPLECRTFFLTQ | [symbol] | skgdvpvirepfiscsplecrtffltq | |
| [symbol] | GALLNDKHSNGTIKDRSPYRTLMSVPI | 165S, | gallndkhsngtikdrspyrtlmsVpi | |
| [symbol] | GSVRSRYNARFESIAWSASACHDGINW | 100L/408M/419V, | gSvrsrynArfesIawsasachdginw | |
| | LTIGITGPDNFAVAVLKYNGIITDTIK | 161V/172A, | ltigiTgpdnfavavlkyngiitdtik | |
| | SWRNMILRTQESECACVNGSCFTVMTG | | swrnsilrtqesecacvngscftvmtg | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 48) | | aelpftldk (SEQ ID NO: 48) | |
| 401_N1- | VKLAGNSSLCPVSGWAILSKDNSVRIG | N1 numbering: | vklagnsslcpvsgwaILskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 100L/408M/419V, | gallndkhsngtikdrspyrtlmsVpi | |
| [symbol] | GSVRSRYNARFESIAWSASACHDGINW | 161V/172A, | gSvrsrynArfesIawsasachdginw | |
| [symbol] | LTIGITGPDNFAVAVLKYNGIITDTIK | | ltigiTgpdnfavavlkyngiitdtik | |
| | SWRNMILRTQESECACVNGSCFTVMTG | | swrnsilrtqesecacvngscftvmtg | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 49) | | aelpftldk (SEQ ID NO: 49) | |
| 402_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSVRSRYNARFESIAWSASACHDGINW | | gSvrsrynArfesIawsasachdginw | |
| [symbol] | LTIGITGPDNFAVAVLKYNGIITDTIK | | ltigiTgpdnfavavlkyngiitdtik | |
| [symbol] | SWRNMILRTQESECACVNGSCFTVMTG | | swrnsilrtqesecacvngscftvmtg | |
| [symbol] | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 50) | | aelpftldk (SEQ ID NO: 50) | |
| 403_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSPPSPYNARFESVAWSASACHDGINW | | gSPpspynArfesvawsasachdginw | |
| [symbol] | LTIGITGPDNFAVAVLKYNGIITDTIK | | ltigiTgpdnfavavlkyngiitdtik | |
| [symbol] | SWRNMILRTQESECACVNGSCFTVMTG | | swrnsilrtqesecacvngscftvmtg | |
| [symbol] | GPSNGQASYKIFRIEKGKIVKSVEMNA | | gpsngqasykifriekgkivksvemna | |
| [symbol] | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 51) | | aelpftldk (SEQ ID NO: 51) | |
| 404_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSPPSPYNARFESVAWSASACHDGINW | [symbol] | gSPpspynArfesvawsasachdginw | |
| [symbol] | LTIGITGPDNFAVAVLKYNGIITDTIK | | ltigiTgpdnfavavlkyngiitdtik | |
| [symbol] | SWRNMILRTQESECACVNGSCFTVMTG | | swrnsilrtqesecacvngscftvmtg | |
| [symbol] | GPSNGQASYKIFRIEKGKIVKSVEMNA | | gpsngqasykifriekgkivksvemna | |
| [symbol] | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 52) | | aelpftldk (SEQ ID NO: 52) | |
| 405_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSPPSPYNARFESVAWSASACHDGINW | [symbol] | gSPpspynArfesvawsasachdginw | |
| [symbol] | LTIGITGPDNFAVAVLKYNGIITDTIK | [symbol] | ltigiTgpdnfavavlkyngiitdtik | |
| [symbol] | SWRNMILRTQESECACVNGSCFTVMTG | | swrnsilrtqesecacvngscftvmtg | |
| [symbol] | GPSNGQASYKIFRIEKGKIVKSVEMNA | | gpsngqasykifriekgkivksvemna | |
| [symbol] | PNYHYEECSCYPDSSEITCVCRDNWKG | | pnyhyeecscypdsseitcvcrdnwkg | |
| [symbol] | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 53) | | aelpftldk (SEQ ID NO: 53) | |
| 406_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSPPSPYNARFESVAWSASACHDGINW | [symbol] | gSPpspynArfesvawsasachdginw | |
| [symbol] | LTIGITGPDNFAVAVLKYNGIITDTIK | [symbol] | ltigiTgpdnfavavlkyngiitdtik | |
| [symbol] | SWRNMILRTQESECACVNGSCFTVMTG | A | swrnsilrtqesecacvngscftvmtg | |
| [symbol] | GPSNGQASYKIFRIEKGKIVKSVEMNA | | gpsngqasykifriekgkivksvemna | |
| [symbol] | PNYHYEECSCYPDSSEITCVCRDNWKG | | pnyhyeecscypdsseitcvcrdnwkg | |
| [symbol] | SNRPWVSEFNQNIEYQIGYCSGIFGDN | | snrpwvsefnqnieyqigycsgifgdn | |
| [symbol] | PRPNDKTGSCGPVSSNGANGVKGFSFK | | prpndktgscgpvssngangvkgfsfk | |
| [symbol] | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 54) | | aelpftldk (SEQ ID NO: 54) | |
| 407_N1- | VKLAGNSSLCPVSGWAIYSKDNSVRIG | N1 numbering: | vklagnsslcpvsgwaiyskdnsvrig | |
| [symbol] | SKGDVPVIREPFISCSPLECRTFFLTQ | 165S, | skgdvpvirepfiscsplecrtffltq | |
| [symbol] | GALLNDKHSNGTIKDRSPYRTLMSVPI | 161V/172A, | gallndkhsngtikdrspyrtlmsVpi | |
| [symbol] | GSPPSPYNARFESVAWSASACHDGINW | 114I/166P, | gSPpspynArfesvawsasachdginw | |
| [symbol] | LTIGITGPDNFAVAVLKYNGIITDTIK | [symbol] | ltigiTgpdnfavavlkyngiitdtik | |
| [symbol] | SWRNMILRTQESECACVNGSCFTVMTG | [symbol] | swrnsilrtqesecacvngscftvmtg | |
| [symbol] | GPSNGQASYKIFRIEKGKIVKSVEMNA | A | gpsngqasykifriekgkivksvemna | |
| [symbol] | PNYHYEECSCYPDSSEITCVCRDNWKG | | pnyhyeecscypdsseitcvcrdnwkg | |
| [symbol] | SNRPWVSEFNQNIEYQIGYCSGIFGDN | | snrpwvsefnqnieyqigycsgifgdn | |
| [symbol] | PRPNDKTGSCGPVSSNGANGVKGFSFK | | prpndktgscgpvssngangvkgfsfk | |
| [symbol] | YGNGVWIGRTKSISSRNGFEMIWDPNG | | ygngvwigrtksissrngfemiwdpng | |
| [symbol] | WTGTCNNFSIRQDIVGINSWSGYSGSF | | wtgtcnnfsirqdivginswsgysgsf | |
| | VGHPELTGLDCIVPCFWVELIRGRPKE | | vghpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 55) | | aelpftldk (SEQ ID NO: 55) | |
| 408_N1- | VKLAGNSSLCPVSGWAPLSKDNSVRIG | N1 numbering: | vklagnsslcpvsgwaPLskdnsvrig | |
| [symbol] | SKGDIPVIREPFISCSPLECRTFFLTQ | [symbol] | skgdIpvirepfiscsplecrtffltq | |
| [symbol] | GALLNDKHSNGTIKDRSPYRTLMSVPI | 205I, 165S | gallndkhsngtikdrspyrtlmsVpi | |
| [symbol] | GSPPSPYNARFESVAWSASACHDGINW | [symbol] | gSPpspynArfesvawsasachdginw | |
| | LTIGITGPDNFAVAVLKYNGIITDTIK | [symbol] | ltigiTgpdnfavavlkyngiitdtik | |
| | SWRNMILRTQESECACVNGSCFTVMTG | [symbol] | swrnsilrtqesecacvngscftvmtg | |
| | GPSNGQASYKIFRIEKGKIVKSVEMNA | 114I/166F | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTCNNFSIRQDIVGINSWSGYSGSF | | wtgtcnnfsirqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 56) | | aelpftldk (SEQ ID NO: 56) | |
| 409_N1- | VKLAGNSSLCPVSGWAPLSKDNSVRIG | N1 numbering: | vklagnsslcpvsgwaPLskdnsvrig | |
| [symbol] | SKGDVFVIREPFISCSPLECRQFFLTQ | [symbol] | skgdvfvirepfiscsplecrQffltq | |
| [symbol] | GALLNDKHSNGTIKDRSPYRTLMSVPI | 205I, 165S | gallndkhsngtikdrspyrtlmsVpi | |
| [symbol] | GSVPSPYNARFESIAWSASACHDGINW | 100L/408M/419V, | gSvpspynArfesIawsasachdginw | |
| | LTIGITGPDNFAVAILKYNGIITDTIK | 161V/172A, | ltigiTgpdnfavaIlkyngiitdtik | |
| | SWRNMILRTQESECACVNGSCFTVMTG | 131Q | swrnsilrtqesecacvngscftvmtg | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 57) | | aelpftldk (SEQ ID NO: 57) | |
| 410_N1- | VKLAGNSSLCPVSGWAPLSKDNSVRIG | N1 numbering: | vklagnsslcpvsgwaPLskdnsvrig | |
| [symbol] | SKGDIFVIREPFISCSPLECRQFFLTQ | [symbol] | skgdIfvirepfiscsplecrQffltq | |
| [symbol] | GALLNDKHSNGTIKDRSPYRTLMSVPI | 205I, 165S | gallndkhsngtikdrspyrtlmsVpi | |
| [symbol] | GSPPSPYNARFESIAWSASACHDGINW | 100L/408M/419V, | gSPpspynArfesIawsasachdginw | |
| [symbol] | LTIGITGPDNFAVAILKYNGIITDTIK | 161V/172A, | ltigiTgpdnfavaIlkyngiitdtik | |
| | SWRNMILRTQESECACVNGSCFTVMTG | [symbol] | swrnsilrtqesecacvngscftvmtg | |
| | GPSNGQASYKIFRIEKGKIVKSVEMNA | 131Q | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 58) | | aelpftldk (SEQ ID NO: 58) | |
| 411_N1- | VKLAGNSSLCPVSGWAPLSKDNSVRIG | N1 numbering: | vklagnsslcpvsgwaPLskdnsvrig | |
| [symbol] | SKGDVFVIREPFISCSPLECRMFFLTQ | [symbol] | skgdvfvirepfiscsplecrMffltq | |
| [symbol] | GALLNDKHSNGTIKDRSPYRTLMSVPI | 205I, 165S | gallndkhsngtikdrspyrtlmsVpi | |
| [symbol] | GSVPSPYNARFESIAWSASACHDGINW | 100L/408M/419V, | gSvpspynArfesIawsasachdginw | |
| [symbol] | LTIGITGPDNFAVAILKYNGIITDTIK | 161V/172A, | ltigiTgpdnfavaIlkyngiitdtik | |
| [symbol] | SWRNMILRTQESECACVNGSCFTVMTG | [symbol] | swrnsilrtqesecacvngscftvmtg | |
| | GPSNGQASYKIFRIEKGKIVKSVEMNA | [symbol] | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 59) | | aelpftldk (SEQ ID NO: 59) | |
| [symbol] | VKLAGNSSLCPVSGWAPLAKDNSVRIG | N1 numbering: | vklagnsslcpvsgwaPLAkdnsvrig | |
| [symbol] | SKGDIFVIREPFISCSPLECRMFFLTQ | [symbol] | skgdIfvirepfiscsplecrMffltq | |
| [symbol] | GALLNDKHSNGTIKDRSPYRTLMSVPI | 205I, 165S | gallndkhsngtikdrspyrtlmsVpi | |
| [symbol] | GSPPSPYNARFESIAWSASACHDGINW | 100L/408M/419V, | gSPpspynArfesIawsasachdginw | |
| [symbol] | LTIGITGPDNFAVAILKYNGIITDTIK | 161V/172A, | ltigiTgpdnfavaIlkyngiitdtik | |
| [symbol] | SWRNMILRTQESECACVNGSCFTVMTG | [symbol] | swrnsilrtqesecacvngscftvmtg | |
| [symbol] | GPSNGQASYKIFRIEKGKIVKSVEMNA | [symbol] | gpsngqasykifriekgkivksvemna | |
| [symbol] | PNYHYEECSCYPDSSEITCVCRDNWKG | [symbol] | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 60) | | aelpftldk (SEQ ID NO: 60) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | 165S | skgdvfvirepfiscsplecrtffltq | |
| [symbol] | GALLNDKHSNGTIKDRSPYRTLMSCPI | | gallndkhsngtikdrspyrtlmscpi | |
| | GSVPSPYNSRFESVAWSASACHDGINW | | gSvpspynsrfesvawsasachdginw | |
| | LTIGISGPDNSAVAVLKYNGIITDTIK | | ltigisgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIRPCFWVELIRGRPKE | | vqhpeltgldcirpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 61) | | aelpftldk (SEQ ID NO: 61) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | 165T | skgdvfvirepfiscsplecrtffltq | |
| [symbol] | GALLNDKHSNGTIKDRSPYRTLMSCPI | | gallndkhsngtikdrspyrtlmscpi | |
| | GTVPSPYNSRFESVAWSASACHDGINW | | gTvpspynsrfesvawsasachdginw | |
| | LTIGISGPDNSAVAVLKYNGIITDTIK | | ltigisgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSYK | | prpndktgscgpvssngangvkgfsYk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIRPCFWVELIRGRPKE | | vqhpeltgldcirpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 62) | | aelpftldk (SEQ ID NO: 62) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | 165V | skgdvfvirepfiscsplecrtffltq | |
| [symbol] | GALLNDKHSNGTIKDRSPYRTLMSCPI | | gallndkhsngtikdrspyrtlmscpi | |
| | GVVPSPYNSRFESVAWSASACHDGINW | | gVvpspynsrfesvawsasachdginw | |
| | LTIGISGPDNSAVAVLKYNGIITDTIK | | ltigisgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSYK | | prpndktgscgpvssngangvkgfsYk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIRPCFWVELIRGRPKE | | vqhpeltgldcirpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 63) | | aelpftldk (SEQ ID NO: 63) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | 165A | skgdvfvirepfiscsplecrtffltq | |
| [symbol] | GALLNDKHSNGTIKDRSPYRTLMSCPI | | gallndkhsngtikdrspyrtlmscpi | |
| | GAVPSPYNSRFESVAWSASACHDGINW | | gAvpspynsrfesvawsasachdginw | |
| | LTIGISGPDNSAVAVLKYNGIITDTIK | | ltigisgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSYK | | prpndktgscgpvssngangvkgfsYk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIRPCFWVELIRGRPKE | | vqhpeltgldcirpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 64) | | aelpftldk (SEQ ID NO: 64) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | 165T | skgdvfvirepfiscsplecrtffltq | |
| [symbol] | GALLNDKHSNGTIKDRSPYRTLMSCPI | | gallndkhsngtikdrspyrtlmscpi | |
| | GIVPSPYNSRFESVAWSASACHDGINW | | gIvpspynsrfesvawsasachdginw | |
| | LTIGISGPDNSAVAVLKYNGIITDTIK | | ltigisgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSEFNQNIEYQIGYCSGIFGDN | | snrpwvsefnqnieyqigycsgifgdn | |
| | PRPNDKTGSCGPVSSNGANGVKGFSYK | | prpndktgscgpvssngangvkgfsYk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIRPCFWVELIRGRPKE | | vqhpeltgldcirpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 65) | | aelpftldk (SEQ ID NO: 65) | |
| N1- | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | [symbol] | gallndkhsngtikdrspyrtlmsVpi | |
| | GTVPSPYNARFESIAWSASACHDGINW | [symbol] | gTvpspynArfesIawsasachdginw | |
| | LTIGITGPDNSAVAVLKYNGIITDTIK | [symbol] | ltigiTgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 66) | | aelpftldk (SEQ ID NO: 66) | |
| N1-Ca109- | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | [symbol] | gallndkhsngtikdrspyrtlmsVpi | |
| | GVVPSPYNARFESIAWSASACHDGINW | [symbol] | gVvpspynArfesIawsasachdginw | |
| | LTIGITGPDNSAVAVLKYNGIITDTIK | [symbol] | ltigiTgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 67) | | aelpftldk (SEQ ID NO: 67) | |
| N1-Ca109- | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | [symbol] | gallndkhsngtikdrspyrtlmsVpi | |
| | GAVPSPYNARFESIAWSASACHDGINW | [symbol] | gAvpspynArfesIawsasachdginw | |
| | LTIGITGPDNSAVAILKYNGIITDTIK | [symbol] | ltigiTgpdnsavaIlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 68) | | aelpftldk (SEQ ID NO: 68) | |
| N1-Ca109- | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | [symbol] | gallndkhsngtikdrspyrtlmsVpi | |
| | GIVPSPYNARFESIAWSASACHDGINW | [symbol] | gIvpspynArfesIawsasachdginw | |
| | LTIGITGPDNSAVAILKYNGIITDTIK | [symbol] | ltigiTgpdnsavaIlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 69) | | aelpftldk (SEQ ID NO: 69) | |
| N1- | VKLAGNSSLCPVSGWAIYSKDNSVRIG | N1 numbering: | vklagnsslcpvsgwaiyskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | | gallndkhsngtikdrspyrtlmsVpi | |
| | GEVPSPYNARFESVAWSASACHDGINW | | gevpspynArfesvawsasachdginw | |
| | LTIGISGPDNGAVAVLKYNGIITDTIK | | ltigisgpdngavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VQHPELTGLDCIVPCFWVELIRGRPKE | | vqhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 70) | | aelpftldk (SEQ ID NO: 70) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRTFFLTQ | [symbol] | skgdvfvirepfVscsplecrtffltq | |
| [symbol] | GALLNDKHSNGTIKDRSPYRTLMSVPI | [symbol] | gallndkhsngtikdrspyrtlmsVpi | |
| [symbol] | CSVPSPYNARVESIAWSASACHDGINW | [symbol] | CSvpspynArVesIawsasachdginw | |
| [symbol] | LTIGITGPDNSAVAILKYNGIITDTIK | [symbol] | ltigiTgpdnsavaIlkyngiitdtik | |
| | SWRNNILRTQESECACVNGSCFTVMTD | [symbol] | swrnnilrtqesecacvngscftvmtd | |
| | GPSNGQASYKIFRIEKGKIVKSVEMNA | [symbol] | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 71) | | aelpftldk (SEQ ID NO: 71) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRTFFLTQ | [symbol] | skgdvfvirepfVscsplecrtffltq | |
| [symbol] | GALLNDKHSNGTIKDRSPYRTLMSVPI | [symbol] | gallndkhsngtikdrspyrtlmsVpi | |
| [symbol] | CSPPSPYNARVESIAWSASACHDGINW | [symbol] | CSPpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | 114I/166P, | swrnnilrtqesecacvngscftvmtd | |
| [symbol] | GPSNGQASYKIFRIEKGKIVKSVEMNA | [symbol] | gpsngqasykifriekgkivksvemna | |
| | PNYHYEECSCYPDSSEITCVCRDNWKG | [symbol] | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 72) | | aelpftldk (SEQ ID NO: 72) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRQFFLTQ | [symbol] | skgdvfvirepfVscsplecrQffltq | |
| [symbol] | GALLNDKHSNGTIKDRSPYRTLMSVPI | [symbol] | gallndkhsngtikdrspyrtlmsVpi | |
| [symbol] | CSVPSPYNARVESIAWSASACHDGINW | [symbol] | CSvpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| | GPSNGQASYKIFRIEKGKIVKSVEMNA | [symbol] | gpsngqasykifriekgkivksvemna | |
| | PNYHYEECSCYPDSSEITCVCRDNWKG | [symbol] | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 73) | | aelpftldk (SEQ ID NO: 73) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRQFFLTQ | [symbol] | skgdvfvirepfVscsplecrQffltq | |
| [symbol] | GALLNDKHSNGTIKDRSPYRTLMSVPI | [symbol] | gallndkhsngtikdrspyrtlmsVpi | |
| [symbol] | CSPPSPYNARVESIAWSASACHDGINW | [symbol] | CSPpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| [symbol] | GPSNGQASYKIFRIEKGKIVKSVEMNA | [symbol] | gpsngqasykifriekgkivksvemna | |
| | PNYHYEECSCYPDSSEITCVCRDNWKG | [symbol] | pnyhyeecscypdsseitcvcrdnwkg | |
| | SNRPWVSFNQNLEYQIGYICSGIFGDN | [symbol] | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 74) | | aelpftldk (SEQ ID NO: 74) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRMFFLTQ | [symbol] | skgdvfvirepfVscsplecrMffltq | |
| [symbol] | GALLNDKHSNGTIKDRSPYRTLMSVPI | [symbol] | gallndkhsngtikdrspyrtlmsVpi | |
| [symbol] | CSVPSPYNARVESIAWSASACHDGINW | [symbol] | CSvpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| [symbol] | GPSNGQASYKIFRIEKGKIVKSVEMNA | [symbol] | gpsngqasykifriekgkivksvemna | |
| | PNYHYEECSCYPDSSEITCVCRDNWKG | [symbol] | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 75) | | aelpftldk (SEQ ID NO: 75) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRMFFLTQ | [symbol] | skgdvfvirepfVscsplecrMffltq | |
| [symbol] | GALLNDKHSNGTIKDRSPYRTLMSVPI | [symbol] | gallndkhsngtikdrspyrtlmsVpi | |
| [symbol] | CSVPSPYNARVESIAWSASACHDGINW | [symbol] | CSvpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| [symbol] | GPSNGQASYKIFRIEKGKIVKSVEMNA | [symbol] | gpsngqasykifriekgkivksvemna | |
| | PNYHYEECSCYPDSSEITCVCRDNWKG | [symbol] | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSIIVFCGVNSDTVGWSWPDG | | ntiwtsgsIiVfcgvnsdtvgwswpdg | |
| | AELPFTLDK (SEQ ID NO: 76) | | aelpftldk (SEQ ID NO: 76) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRMFFLTQ | [symbol] | skgdvfvirepfVscsplecrMffltq | |
| [symbol] | GALLNDKHSNGTIKDRSPYRTLMSVPI | [symbol] | gallndkhsngtikdrspyrtlmsVpi | |
| [symbol] | CSPPSPYNARVESIAWSASACHDGINW | [symbol] | CSPpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| [symbol] | GPSNGQASYKIFRIEKGKIVKSVEMNA | [symbol] | gpsngqasykifriekgkivksvemna | |
| [symbol] | PNYHYEECSCYPDSSEITCVCRDNWKG | [symbol] | pnyhyeecscypdsseitcvcrdnwkg | |
| | SNRPWVSFNQNLEYQIGYICSGIFGDN | [symbol] | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSIIAFCGVNSDTVGWSWPDG | | ntiwtsgsIiAfcgvnsdtvgwswpdg | |
| | AELPFTLDK (SEQ ID NO: 77) | | aelpftldk (SEQ ID NO: 77) | |
| N1-Ca109- | VKLAGNSSLCPVSGWAPLCKDNSVRIG | N1 numbering: | vklagnsslcpvsgwaPLCkdnsvrig | |
| [symbol] | SKGDVFVIREPFVSCSPLECRMFFLTQ | [symbol] | skgdvfvirepfVscsplecrMffltq | |
| [symbol] | GALLNDKHSNGTIKDRSPYRTLMSVPI | [symbol] | gallndkhsngtikdrspyrtlmsVpi | |
| [symbol] | CSPPSPYNARVESIAWSASACHDGINW | [symbol] | CSPpspynArVesIawsasachdginw | |
| [symbol] | LTIGITGPDNSAVAILKYNGIITDTIK | [symbol] | ltigiTgpdnsavaIlkyngiitdtik | |
| [symbol] | SWRNNILRTQESECACVNGSCFTVMTD | [symbol] | swrnnilrtqesecacvngscftvmtd | |
| [symbol] | GPSNGQASYKIFRIEKGKIVKSVEMNA | [symbol] | gpsngqasykifriekgkivksvemna | |
| [symbol] | PNYHYEECSCYPDSSEITCVCRDNWKG | [symbol] | pnyhyeecscypdsseitcvcrdnwkg | |
| | SNRPWVSFNQNLEYQIGYICSGIFGDN | [symbol] | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSIIVFCGVNSDTVGWSWPDG | | ntiwtsgsIiVfcgvnsdtvgwswpdg | |
| | AELPFTLDK (SEQ ID NO: 78) | | aelpftldk (SEQ ID NO: 78) | |
| N1-Ca109- | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| [symbol] | GALLNDKHSNGTIKDRSPYRTLMSVPI | [symbol] | gallndkhsngtikdrspyrtlmsVpi | |
| | GSVPSPYNARFESIAWSASACHDGINW | [symbol] | gSvpspynArfesIawsasachdginw | |
| | LTIGITGPDNSAVAVLKYNGIITDTIK | [symbol] | ltigiTgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgsIiVfcgvnsdtvgwswpdg | |
| | AELPFTLDK (SEQ ID NO: 87) | | aelpftldk (SEQ ID NO: 87) | |
| [symbol] | VKLAGNSSLCPVSGWAPLSKDNSVRIG | N1 numbering: | vklagnsslcpvsgwaPLskdnsvrig | |
| [symbol] | SKGDVFVIREPFISCSPLECRTFFLTQ | [symbol] | skgdvfvirepfiscsplecrtffltq | |
| | GALLNDKHSNGTIKDRSPYRTLMSVPI | [symbol] | gallndkhsngtikdrspyrtlmsVpi | |
| | GSVPSPYNARFESIAWSASACHDGINW | [symbol] | gSvpspynArfesIawsasachdginw | |
| | LTIGITGPDNSAVAVLKYNGIITDTIK | [symbol] | ltigiTgpdnsavavlkyngiitdtik | |
| | SWRNNILRTQESECACVNGSCFTVMTD | | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNNFSIKQDIVGINSWSGYSGSF | | wtgtdnnfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 88) | | aelpftldk (SEQ ID NO: 88) | |
| N1- | VKLAGNSSLCPVSGWAPLSKDNSVRIG | N1 numbering: | vklagnsslcpvsgwaPLskdnsvrig | |
| [symbol] | SKGDVFVIREPFISCSPLECRQFFLTQ | [symbol] | skgdvfvirepfiscsplecrQffltq | |
| [symbol] | GALLNDKHSNGTIKDRSPYRTLMSVPI | [symbol] | gallndkhsngtikdrspyrtlmsVpi | |
| [symbol] | GSVPSPYNARFESIAWSASACHDGINW | [symbol] | gSvpspynArfesIawsasachdginw | |
| | LTIGITGPDNSAVAVLKYNGIITDTIK | [symbol] | ltigiTgpdnsavavlkyngiitdtik | |
| | SWRNNILRTQESECACVNGSCFTVMTD | [symbol] | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNKFSIKQDIVGINSWSGYSGSF | | wtgtdnkfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 89) | | aelpftldk (SEQ ID NO: 89) | |
| N1- | VKLAGNSSLCPVSGWAPLSKDNSVRIG | N1 numbering: | vklagnsslcpvsgwaPLskdnsvrig | |
| [symbol] | SKGDVFVIREPFISCSPLECRQFFLTQ | [symbol] | skgdvfvirepfiscsplecrQffltq | |
| [symbol] | GALLNDKHSNGTIKDRSPYRTLMSVPI | [symbol] | gallndkhsngtikdrspyrtlmsVpi | |
| [symbol] | GSVPSPYNARFESIAWSASACHDGINW | [symbol] | gSvpspynArfesIawsasachdginw | |
| [symbol] | LTIGITGPDNSAVAVLKYNGIITDTIK | [symbol] | ltigiTgpdnsavavlkyngiitdtik | |
| [symbol] | SWRNNILRTQESECACVNGSCFTVMTD | [symbol] | swrnnilrtqesecacvngscftvmtd | |
| | GPSNGQASYKIFRIEKGKIVKSVEMNA | | gpsngqasykifriekgkivksvemna | |
| | PNYHYEECSCYPDSSEITCVCRDNWKG | | pnyhyeecscypdsseitcvcrdnwkg | |
| | SNRPWVSFNQNLEYQIGYICSGIFGDN | | snrpwvsfnqnleyqigyicsgifgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNKFSIKQDIVGINSWSGYSGSF | | wtgtdnkfsikqdivginswsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 89) | | aelpftldk (SEQ ID NO: 89) | |
| N1- | VKLAGNSSLCPVSGWAPLAKDNSVRIG | N1 numbering: | vklagnsslcpvsgwaPLAkdnsvrig | |
| [symbol] | SKGDVFVIREPFISCSPLECRMFFLTQ | [symbol] | skgdvfvirepfiscsplecrMffltq | |
| [symbol] | GALLNDKHSNGTIKDRSPYRTLMSVPI | [symbol] | gallndkhsngtikdrspyrtlmsVpi | |
| [symbol] | GSVPSPYNARFESIAWSASACHDGINW | [symbol] | gSvpspynArfesIawsasachdginw | |
| [symbol] | LTIGITGPDNSAVAVLKYNGIITDTIK | [symbol] | ltigiTgpdnsavavlkyngiitdtik | |
| [symbol] | SWRNNILRTQESECACVNGSCFTVMTD | [symbol] | swrnnilrtqesecacvngscftvmtd | |
| | GPSNGQASYKIFRIEKGKIIKSVEMKA | [symbol] | gpsngqasykifriekgkiiksvemka | |
| | PNYHYEECSCYPDSSEITCVCRDNWKG | | pnyhyeecscypdsseitcvcrdnwkg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTGTDNKFSIKQDIVGINSWSGYSGSF | | wtgtdnkfsikqdivginswsgysgsf | |
| | VMHPELTGLDCIVPCFWVELIRGRPEE | | vMhpeltgldciVpcfwvelirgrpee | |
| | NTIWTSGSIIAFCGVNSDTVGWSWPDG | | ntiwtsgsIiAfcgvnsdtvgwswpdg | |
| | AELPFTLDK (SEQ ID NO: 90) | | aelpftldk (SEQ ID NO: 90) | |
| N1- | VKLAGNSSLCPINGWAPLSKDNSVRIG | N1 numbering: | vklagnsslcpingwaPLskdnsVrig | |
| [symbol] | SKGDVFVIREPFISCSHLECRQFFLTQ | [symbol] | skgdvfvirepfiscshlecrQffltq | |
| [symbol] | GALLNDKHSNGTIKDRSPYRTLMSVPI | [symbol] | gallndkhsngtikdrspyrtlmsVpi | |
| [symbol] | GSVPSPYNARFESIAWSASACHDGTSW | [symbol] | gSVpspynArfesIawsasachdgtsw | |
| [symbol] | LTIGITGPDNGAVAVLKYNGIITDTIK | [symbol] | ltigiTgpdngavavlkyngiitdtik | |
| [symbol] | SWRNNILRTQESECACVNGSCFTVMTD | [symbol] | swrnnilrtqesecacvngscftvmtd | |
| | GPSNGQASYKIFKMEKGKVVKSVELDA | [symbol] | gpsngqasykifkmekgkvvksvelda | |
| | PNYHYEECSCYPDSSEITCVCRDNWKG | | pnyhyeecscypdsseitcvcrdnwkg | |
| | SNRPWVSFNQNLEYQMGYICSGVFGDN | | snrpwvsfnqnleyqmgyicsgvfgdn | |
| | PRPNDKTGSCGPVSSNGANGVKGFSFK | | prpndktgscgpvssngangvkgfsfk | |
| | YGNGVWIGRTKSISSRNGFEMIWDPNG | | ygngvwigrtksissrngfemiwdpng | |
| | WTETDNKFSVKQDIVAITDWSGYSGSF | | wtetdnkfsvkqdivaitdwsgysgsf | |
| | VMHPELTGLDCIVPCFWVELIRGRPKE | | vMhpeltgldciVpcfwvelirgrpke | |
| | STIWTSGSSISFCGVNSDTVGWSWPDG | | stiwtsgssisfcgvnsdtvgwswpdg | |
| | AELPFTLDK (SEQ ID NO: 91) | | aelpftldk (SEQ ID NO: 91) | |
| N1- | VILTGNSSLCPIRGWAPLSKDNSVRIG | N1 numbering: | viltgnsslcpirgwaPLskdnsVrig | |
| [symbol] | SKGDVFVIREPFISCSHLECRTFFLTQ | [symbol] | skgdvfvirepfiscshlecrtffltq | |
| [symbol] | GALLNDKHSNGTIKDRSPYRTLMSVPI | [symbol] | gallndkhsngtikdrspyrTlmsVpi | |
| [symbol] | GSVPSPYNARFESIAWSASACHDGMGW | [symbol] | gSVpspynArfesIawsasachdgmgw | |
| [symbol] | LTIGITGPDNGAVAVLKYNGIITETIK | [symbol] | ltigiTgpdngavavlkynGiitetik | |
| [symbol] | SWRKNILRTQESECTCVNGSCFTIMTD | [symbol] | swrknilrtqesectcvngscftimtd | |
| [symbol] | GPSNGLASYKIFKIEKGKVTKSIELNA | [symbol] | gpsnglasykifkiekgkvtksielna | |
| | PNYHYEECSCYPDTGKVMCVCRDNWHG | [symbol] | pnyhyeecscypdtgkvmcvcrdnwhg | |
| | SNRPWVSFDQNLDYKIGYICSGVFGDN | | snrpwvsfdqnldykigyicsgvfgdn | |
| | PRPKDGTGSCGPVSSDGANGVKGFSYK | | prpkdgtgscgpvssdgangvkgfsyk | |
| | YGNGVWIGRTKSDSSRHGFEMIWDPNG | | ygngvwigrtksdssrhgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGRPEE | | vMhpeltgldcmVpcfwvelirgrpee | |
| | DAIWTSGSIISFCGVNSDTVDWSWPDG | | daiwtsgsiisfcgvnsdtvdwswpdg | |
| | AELPFTLDK (SEQ ID NO: 92) | | aelpftldk (SEQ ID NO: 92) | |
| N1- | VILTGNSSLCPIRGWAPLSKDNSVRIG | N1 numbering: | viltgnsslcpirgwaPLskdnSVrig | |
| [symbol] | SKGDVFVIREPFISCSHLECRQFFLTQ | [symbol] | skgdvfvirepfiscshlecrQffltq | |
| [symbol] | GALLNDKHSNGTIKDRSPYRTLMSVPI | [symbol] | gallndkhsngtikdrspyrTlmsVpI | |
| [symbol] | GSVPSPYNARFESIAWSASACHDGMGW | [symbol] | gSVpspynArfesIawsasachdgmgw | |
| [symbol] | LTIGITGPDNGAVAVLKYNGIITETIK | [symbol] | ltigiTgpdngavavlkynGiitetik | |
| [symbol] | SWRKNILRTQESECTCVNGSCFTIMTD | [symbol] | swrknilrtqesectcvngscftimtd | |
| [symbol] | GPSDGLASYKIFKIEKGKVTKSIELNA | [symbol] | gpsdglasykifkiekgkvtksielna | |
| [symbol] | PNSHYEECSCYPDTGKVMCVCRDNWHG | [symbol] | pnshyeecscypdtgkvmcvcrdnwhg | |
| | SNRPWVSFDQNLDYKIGYICSGVFGDN | [symbol] | snrpwvsfdqnldykigyicsgvfgdn | |
| | PRPKDGTGSCGPVSADGANGVKGFSYK | | prpkdgtgscgpvsadgangvkgfsyk | |
| | YGNGVWIGRTKSDSSRHGFEMIWDPNG | | ygngvwigrtksdssrhgfemiwdpng | |
| | WTETDSRFSMRQDVVAITNRSGYSGSF | | wtetdsrfsmrqdvvaitnrsgysgsf | |
| | VMHPELTGLDCMVPCFWVELIRGRPEE | | vMhpeltgldcmVpcfwvelirgrpee | |
| | DAIWTSGSIISFCGVNSDTVDWSWPDG | | daiwtsgsiisfcgvnsdtvdwswpdg | |
| | AELPFTLDK (SEQ ID NO: 93) | | aelpftldk (SEQ ID NO: 93) | |
| N1- [symbol] | VKLAGNSSLCPVSGWAPLSKDNSVRIG | N1 numbering: | vklagnsslcpvsgwaPLskdnsvrig | |
| [symbol] | SKGSVFVIREPFISCSHLECRQFFLTQ | [symbol] | skgsvfvirepfiscshlecrQffltq | |
| [symbol] | GALLNDKHSNGTIKDRSPYRTLMSVPI | [symbol] | gallndkhsngtikdrspyrTlmsVpI | |
| | GSVPSPYNARFESIAWSASACHDGINW | [symbol] | gSvpspynArfesIawsasachdginw | |
| | LTIGITGPDNGAVAVLKYNGIITDTIK | [symbol] | ltigiTgpdngavavlkyngiitdtik | |
| | SWRKNILRTQESECTCVNGSCFTVMTD | [symbol] | swrknilrtqesectcvngscftvmtd | |
| | GPSDGQASYKIFRIEKGKVTKSVEMNA | | gpsdgqasykifriekgkvtksvemna | |
| | PNSHYEECSCYPDSSEITCVCRDNWHG | | pnshyeecscypdsseitcvcrdnwhg | |
| | SNRPWVSFDQNLDYKIGYICSGVFGDN | | snrpwvsfdqnldykigyicsgvfgdn | |
| | PRPKDGTGSCGPVSADGANGVKGFSYK | | prpkdgtgscgpvsadgangvkgfsyk | |
| | YGNGVWIGRTKSISSRHGFEMIWDPNG | | ygngvwigrtksissrhgfemiwdpng | |
| | WTGTDNNFSIRQDIVGINEWSGYSGSF | | wtgtdnnfsirqdivginewsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 94) | | aelpftldk (SEQ ID NO: 94) | |
| N1-Ca109- | VKLAGNSSLCPVSGWAPLSKDNSVRIG | N1 numbering: | vklagnsslcpvsgwaPLskdnsvrig | |
| [symbol] | SKGSVFVIREPFISCSHLECRMFFLTQ | [symbol] | skgsvfvirepfiscshlecrMffltq | |
| [symbol] | GALLNDKHSNGTIKDRSPYRTLMSVPI | 165S, | gallndkhsngtikdrspyrtlmsVpI | |
| [symbol] | GSVPSPYNARFESIAWSASACHDGINW | [symbol] | gSvpspynArfesIawsasachdginw | |
| [symbol] | LTIGITGPDNGAVAVLKYNGIITDTIK | [symbol] | ltigiTgpdngavavlkyngiitdtik | |
| [symbol] | SWRKNILRTQESECTCVNGSCFTVMTD | [symbol] | swrknilrtqesectcvngscftvmtd | |
| | GPSDGQASYKIFRIEKGKVTKSVEMNA | [symbol] | gpsdgqasykifriekgkvtksvemna | |
| | PNSHYEECSCYPDSSEITCVCRDNWHG | | pnshyeecscypdsseitcvcrdnwhg | |
| | SNRPWVSFDQNLDYKIGYICSGVFGDN | | snrpwvsfdqnldykigyicsgvfgdn | |
| | PRPKDGTGSCGPVSADGANGVKGFSYK | | prpkdgtgscgpvsadgangvkgfsyk | |
| | YGNGVWIGRTKSISSRHGFEMIWDPNG | | ygngvwigrtksissrhgfemiwdpng | |
| | WTGTDNNFSIRQDIVGINEWSGYSGSF | | wtgtdnnfsirqdivginewsgysgsf | |
| | VMHPELTGLDCIVPCFWVELIRGRPKE | | vMhpeltgldciVpcfwvelirgrpke | |
| | NTIWTSGSSISFCGVNSDTVGWSWPDG | | ntiwtsgssisfcgvnsdtvgwswpdg | |
| | AELPFTLDK (SEQ ID NO: 95) | | aelpftldk (SEQ ID NO: 95) | |

[symbol]

indicates data missing or illegible when filed

We claim:

1. A non-naturally occurring mutant neuraminidase (NA) polypeptide that improves expression and/or modifies the open/closed tetrameric conformational state of the NA polypeptide.

2. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 1, 2, 3, 4, 5, 6, or all 7 of the following amino acid residues relative to SEQ ID NO:1 when aligned by protocol 1 or protocol 2:
161T/A, 105A, 165T/I, 166P, 196Q, 203Y, 444V; or
(b) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 2, 3, 4, 5, 6, 7, 8, 9, 10, 11, 12, 13, 14, 15, 16, or all 17 of the following amino acid residues relative to SEQ ID NO:1when aligned by protocol 1 or protocol 2:
161T/A/V/S/T, 100L, 408M, 419V, 99P, 103N, 105A, 131Q/M, 163I/L, 165T/S/V/A/I, 166P, 177I, 196Q/T, 203Y, 205I, 442I, 444V.

3. The polypeptide of claim 2, wherein the polypeptide is
(a) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 1, 2, 3, 4, 5, 6, or all 7 of the following amino acid residues relative to SEQ ID NO: 1 when aligned by protocol 1 or protocol 2:
(i) 161T/A, 105A, 157K, 165T/I, 166P, 196Q, 203Y, 444V; or
(ii) 105A, 165T/I, 166P, 196Q, 203Y, 444V.

4. The polypeptide of claim 2, wherein the polypeptide is
(b) at least 75%, 80%, 85%, 90%, 91%, 92%, 93%, 94%, 95%, 96%, 97%, 98%, or 99% identical to the N1 NA polypeptide of SEQ ID NO: 1, and wherein the non-naturally occurring polypeptide includes 2, 3, 4, 5, 6, 7, 8, 9, 10, 11, 12, 13, 14, 15, 16, or all 17 of the following amino acid residues relative to SEQ ID NO:1 when aligned by protocol 1 or protocol 2:
(i) 161T/A/V/S/T, 100L, 408M, 419V, 99P, 103N, 105A, 131Q/M, 163I/L, 165T/S/V/A/I, 166P, 177I, 196Q/T, 203Y, 205I, 442I, 444V; or
(ii) 99P, 103N, 105A, 131Q/M, 163I/L, 165T/S/V/A/I, 166P, 177I, 196Q/T, 203Y, 205I, 442I, 444V.

5. The polypeptide of any one of claim 2-4, wherein the polypeptide includes one or more of the following sets of amino acid residues relative to SEQ ID NO: 1 when aligned by protocol 1 or protocol 2:
(i)
161V/172A;
100L/408M;
100L/408M/419V
103N/105A;
114I/166P;
101C/164C;
101C/164C/174V;
101C/122V/164C/174V/444V;
122V/444V;
131Q/442I;
101A/163L/444A;
131M/442I;
101A/131M/163L/442I/444A;
100L/101A/131M/163L/442I/444A
131M/163L/442I/444A;
100L/131M/163L/442I/444A;
99P/100L/161V/165S/172A/177I/196T/205I/408M/419V;
99P/100L/161V/165S/172A/177I/196T/408M/419V;
99P/100L/131Q/161V/165S/172A/177I/196T/205I/408M/419V;
99P/100L/131Q/161V/165S/172A/177I/196T/408M/419V;
99P/100L/101A/131M/161V/163I/165S/172A/177I/196T/205I/408M/419V/442I/444A;
99P/100L/101A/131M/161V/163I/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T;
99P/101A/131M/161V/163L/165S/172A/177I/196T/205I/442I/444A;
99P/101A/131M/161V/163L/165S/172A/177I/196T/442I/444A;
99P/196T;
99P/196T/205I;
99P/177I/196T; and/or
99P/177I/196T/205I; or
(ii)
103N/105A;
114I/166P;
101C/164C;
101C/164C/174V;
101C/122V/164C/174V/444V;
122V/444V;
131Q/442I;
101A/I163L/444A;
131M/442I;
101A/131M/163L/442I/444A;
100L/101A/131M/163L/442I/444A;
131M/163L/442I/444A;
100L/131M/163L/442I/444A;
99P/100L/161V/165S/172A/177I/196T/205I/408M/419V;
99P/100L/131Q/161V/165S/172A/177I/196T/205I/408M/419V;
99P/100L/131Q/161V/165S/172A/177I/196T/419V;
99P/100L/101A/131M/161V/163L/165S/172A/177I/196T/205I/408M/419V/442I/444A;
99P/100L/101A/131M/161V/163L/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T;
99P/101A/131M/161V/163L/165S/172A/177I/196T/205I/442I/444A;
99P/101A/131M-161V/163L/165S/172A/177I/196T/442I/444A;
99P/196T;
99P/196T/205I;
99P/177I/196T; and or
99P/177I/196T/205I.

6. The polypeptide of any one of claim 2-4, wherein the polypeptide includes three or more of the listed amino acid residues relative to SEQ ID NO:1 when aligned by protocol 1 or protocol 2.

7. The polypeptide of any one of claim 2-4, wherein the polypeptide includes five or more of the listed amino acid residues relative to SEQ ID NO:1 when aligned by protocol 1 or protocol 2.

8. The polypeptide of any one of claim 2-4, wherein the polypeptide includes seven or more of the listed amino acid residues relative to SEQ ID NO:1when aligned by protocol 1 or protocol 2.

9. The polypeptide of any one of claim 2-4, wherein the polypeptide includes nine or more of the listed amino acid residues relative to SEQ ID NO:1 when aligned by protocol 1 or protocol 2.

10. The polypeptide of any one of claim 2-9, wherein the polypeptide further comprises 1, 2, 3, 4, 5, or all 6 of the following residues relative to SEQ ID NO:1 when aligned by protocol 1 or protocol 2: 105S, 106V, 163I, 166V, 210G, 442S.

11. The polypeptide of any one of claim 2-10, wherein the polypeptide includes one or more of the following sets of ammo acid residues relative to SEQ ID NO: 1 when aligned by protocol 1 or protocol 2:
99P/100L/161V/165S/172A/177I/196T/205I/408M/419V;
99P/100L/161V/165S/172A/177I/196T/408M/419V;
99P/100L/131Q/161V/165S/172A/177I/196T/205I/408M/419V;
99P/100L/131Q/161V/165S/172A/177I/196T/408M/419V;
99P/100L/101A/131M/161V/163L/165S/172A/177I/196T/205I/408M/419V/442I/444A;
99P/100L/101A/131M/161V/163L/165S/172A/177I/196T/408M/419V/442I/444A;
99P/161V/165S/172A/177I/196T/205I;
99P/161V/165S/172A/177I/196T;
99P/131Q/161V/165S/172A/177I/196T/205I;
99P/131Q/161V/165S/172A/177I/196T/;
99P/101A/131M/161V/163L/165S/172A/177I/196T/205I/442I/444A; and/or
99P/101A/131M/161V/163L/165S/172A/177I/196T/442I/444A.

12. The non-naturally occurring polypeptide of any one of claims 2-11, wherein the polypeptide comprises the amino acid sequence at least 75%, 80%, 85%, 90%, 91%, 92%, 93%, 94%, 95%, 96%, 97%, 98%, or 99% identical to the amino acid selected from the group consisting of N1 mutants listed in Table 1 (SEQ ID NO: 10-32 and 39-95), when aligned by protocol 1 or protocol 2, wherein the polypeptide includes all of the residues listed in Table 1 for an individual N1 mutant listed in Table 1.

13. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 1, 2, 3, 4, 5, 6, 7, or all 8 of the following amino acid residues relative to SEQ ID NO:2 when aligned by protocol 1 or protocol 2:
101C, 131M, 162P, 165S/T, 166V, 195Q, 202Y, 443S; or
(b) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 2, 3, 4, 5, 6, 7, 8, 9, 10, 11, or all 12 of the following amino acid residues relative to SEQ ID NO:2 when aligned by protocol 1 or protocol 2:
101 C/A, 103N, 105A, 106V, 131M, 162P, 163I, 165S/T/A/I, 166V, 195Q, 202Y, 443S.

14. The polypeptide of claim 13, wherein the polypeptide is
(a) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 1, 2, 3, 4, 5, 6, 7, or all 8 of the following ammo acid residues relative to SEQ ID NO:2 when aligned by protocol 1 or protocol 2:
101C, 131M, 162P, 165S/T, 166 V, 195Q, 202Y, 443S.

15. The polypeptide of claim 13, wherein the polypeptide is
(b) at least 75%, 80%, 85%, 90%, 91%, 92%, 93%, 94%, 95%, 96%, 97%, 98%, or 99% identical to the N2 NA polypeptide of SEQ ID NO:2, and wherein the non-naturally occurring polypeptide includes 2, 3, 4, 5, 6, 7, 8, 9, 10, 11, or all 12 of the following amino acid residues relative to SEQ ID NO:2 when aligned by protocol 1 or protocol 2:
101C/A, 103N, 105A, 106V, 131M, 162P, 163I, 165S/T/A/I, 166V, 195Q, 202Y, 443S.

16. The polypeptide of any one of claim 13-15, wherein the polypeptide includes one or more of the following sets of amino acid residues relative to SEQ ID NO: 2 when aligned by protocol 1 or protocol 2:
103N/105A;
101C/164C;
101C/164C/173V;
101A/131M;
100L/101A/131M, and/or
100L/131M/163L.

17. The polypeptide of any one of claim 13-15, wherein the polypeptide includes three or more of the listed amino acid residues relative to SEQ ID NO:2 when aligned by protocol 1 or protocol 2.

18. The polypeptide of any one of claim 13-15, wherein the polypeptide includes five or more of the listed amino acid residues relative to SEQ ID NO:2 when aligned by protocol 1 or protocol 2.

19. The polypeptide of any one of claim 13-15, wherein the polypeptide includes seven or more of the listed amino acid residues relative to SEQ ID NO:2 when aligned by protocol 1 or protocol 2.

20. The polypeptide of any one of claim 13-15, wherein the polypeptide includes nine or more of the listed amino acid residues relative to SEQ ID NO:2 when aligned by protocol 1 or protocol 2.

21. The non-naturally occurring polypeptide of any one of claims 13-20, wherein the polypeptide comprises the ammo acid sequence at least 75%, 80%, 85%, 90%, 91%, 92%, 93%, 94%, 95%, 96%, 97%, 98%, or 99% identical to the amino acid selected from the group consisting of N2 mutants listed in Table 1 (SEQ ID NO:33-34), when aligned by protocol 1 or protocol 2, wherein the polypeptide includes all of the residues listed in Table 1 for an individual N2 mutant listed in Table 1.

22. The non-naturally occurring polypeptide of claim 1, wherein the polypeptide is at least 75%, 80%, 85%, 90%, 91%, 92%, 93%, 94%, 95%, 96%, 97%, 98%, or 99% identical to the N2 NA polypeptide of SEQ ID NO: 2, and wherein the non-naturally occurring polypeptide includes a 165Q/E amino acid residues relative to SEQ ID NO:2 when aligned by protocol 1 or protocol 2, or includes 2 or 3 of 165Q/E, 176V, 195S amino acid residues relative to SEQ ID NO:2 when aligned by protocol 1 or protocol 2.

23. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 1, 2, 3, 4, 5, 6, 7, 8, 9, 10, or all 11 of the following amino acid residues relative to SEQ ID NO:3 when aligned by protocol 1 or protocol 2:
103N, 105S, 131Q/M, 157T, 165S/I, 166P, 196Q, 203Y, 205I, 443S, 445V; or
(b) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 2, 3, 4, 5, 6, 7, 8, 9, 10, 11, 12, or all 13 of the following amino acid residues relative to SEQ ID NO:1 when aligned by protocol 1 or protocol 2:
103N, 105S, 106V, 131Q/M, 157T, 163L, 165S/I/V/A, 166P/V, 196Q, 203Y, 205I, 443S, 445V.

24. The polypeptide of claim 23, wherein the polypeptide is
(a) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 1, 2, 3, 4, 5, 6,7, 8, 9, 10, or all 11 of the following amino acid residues relative to SEQ ID NO:3 when aligned by protocol 1 or protocol 2:
103N, 105S, 131Q/M, 157T, 165S/I, 166P, 196Q, 203Y, 205I, 443S, 445V.

25. The polypeptide of claim 23, wherein the polypeptide is
(b) at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 2, 3, 4, 5, 6, 7, 8, 9, 10, 11, 12, or all 13 of the following amino acid residues relative to SEQ ID NO:3 when aligned by protocol 1 or protocol 2:
103N, 105S, 106V, 131Q/M, 157T, 163L, 165S/I/V/A, 166P/V, 196Q, 203Y, 205I, 443S, 445V.

26. The polypeptide of any one of claim 23-25, wherein the polypeptide includes one or more of the following sets of amino acid residues relative to SEQ ID NO:3 when aligned by protocol 1 or protocol 2:
101C/164C;
101C/164C/174V;
101C/164C/174V/445V;
101A/445V;
101A/131M/445A;
131M/445A;
114I/166P;
101A/163I/445V;
101A/131M/163L/445A; and/or
131M/163L/445A.

27. The polypeptide of any one of claim 23-25, wherein the polypeptide includes three or more of the listed amino acid residues relative to SEQ ID NO:3 when aligned by protocol 1 or protocol 2.

28. The polypeptide of any one of claim 23-25, wherein the polypeptide includes five or more of the listed amino acid residues relative to SEQ ID NO:3 when aligned by protocol 1 or protocol 2.

29. The polypeptide of any one of claim 23-25, wherein the polypeptide includes seven or more of the listed amino acid residues relative to SEQ ID NO:3 when aligned by protocol 1 or protocol 2.

30. The polypeptide of any one of claim 23-25, wherein the polypeptide includes nine or more of the listed amino acid residues relative to SEQ ID NO:3 when aligned by protocol 1 or protocol 2.

31. The non-naturally occurring polypeptide of claim 1, wherein the poly peptide is at least 75%, 80%, 85%, 90%, 91%, 92%, 93%, 94%, 95%, 96%, 97%, 98%, or 99% identical to the N3 NA polypeptide of SEQ ID NO:3, and wherein the non-naturally occurring polypeptide includes 1, or both of the following amino acid residues relative to SEQ ID NO:3 when aligned by protocol 1 or protocol 2: 196S, 165Q/E.

32. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 1, 2, 3, 4, 5, 6, 7, 8, 9, 10, 11, 12, 13, 14, 15, or all 16 of the following amino acid residues relative to SEQ ID NO: 4 when aligned by protocol 1 or protocol 2:
160V/S/T/A, 409M, 102N, 104S/A, 105V, 112D, 130Q/M, 162L, 164S/T/A/I, 165P, 176I, 195Q, 202Y, 204I, 443I, 445V; or
(b) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 2, 3, 4, 5, 6, 7, 8, 9, 10, 11, 12, 13, 14, 15, or all 16 of the following amino acid residues relative to SEQ ID NO:4 when aligned by protocol 1 or protocol 2:
160V/S/T/A, 409M, 102N, 104S/A, 105V, 112D, 130Q/M, 162L, 164S/T/A/I, 165P/V, 176I, 195Q, 202Y, 204I, 443I, 445V.

33. The polypeptide of claim 32, wherein the polypeptide is
(a) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 1, 2, 3, 4, 5, 6, 7, 8, 9, 10, 11, 12, 13, 14, 15, or all 16 of the following amino acid residues relative to SEQ ID NO:4 when aligned by protocol 1 or protocol 2:
(i) 160V/S/T/A, 409M, 102N, 104S/A, 105V, 112D, 130Q/M, 162L, 164S/T/A/I, 165P, 176I, 195Q, 202Y, 204I, 443I, 445V; or
(ii) 102N, 104S/A, 105V, 112D, 130Q/M, 162L, 164S/T/A/I, 165P, 176I, 195Q, 202Y, 204I, 443I, 445V.

34. The polypeptide of claim 32, wherein the polypeptide is
(b) at least 75%, 80%, 85%, 90%, 91%, 92%, 93%, 94%, 95%, 96%, 97%, 98%, or 99% identical to the N4 NA polypeptide of SEQ ID NO:4, and wherein the non-naturally occurring polypeptide includes 2, 3, 4, 5, 6, 7, 8, 9, 10, 11, 12, 13, 14, 15, or all 16 of the following amino acid residues relative to SEQ ID NO:4 when aligned by protocol 1 or protocol 2:
(i) 160V/S/T/A, 409M, 102N, 104S/A, 105V, 112D, 130Q/M, 162L, 164S/T/A/I, 165P/V, 176I, 195Q, 202Y, 204I, 443I, 445V; or
(ii) 102N, 104S/A, 105V, 112D, 130Q/M, 162L, 164S/T/A/I, 165P/V, 176I, 195Q, 202Y, 204I, 443I, 445V.

35. The polypeptide of any one of claim 32-34, wherein the polypeptide includes one or more of the following sets of amino acid residues relative to SEQ ID NO:4 when aligned by protocol 1 or protocol 2:
(i)
160V/S/T/A;
160V/171A;
102N/104A;
100C/163C;
100C/163C/173V;
100C/163C/173V/445V;
130Q/443I;
100A/162L/445A;
130M/443I;
100A/130M/162L/443I/445A;
130M/162L/443I/445A;
176I/204I;
100C/121V/163C/173V/445V; and/or
121V/445V; or
(ii)
102N/104A;
100C/163C;
100C/163C/173V;
100C/163C/173V/445V;
130Q/443I;
100A/162L/445V;
130M/443I;
100A/130M/162L/443I/445A;
130M/162L/443I/445A;
176I/204I;
100C/121V/163C/173V/445V; and/or
121V/445V.

36. The polypeptide of any one of claim 32-34, wherein the polypeptide includes three or more of the listed amino acid residues relative to SEQ ID NO:4 when aligned by protocol 1 or protocol 2.

37. The polypeptide of any one of claim 32-34, wherein the polypeptide includes five or more of the listed amino acid residues relative to SEQ ID NO:4 when aligned by protocol 1 or protocol 2.

38. The polypeptide of any one of claim 32-34, wherein the polypeptide includes seven or more of the listed amino acid residues relative to SEQ ID NO:4 when aligned by protocol 1 or protocol 2.

39. The polypeptide of any one of claim 32-34, wherein the polypeptide includes nine or more of the listed amino acid residues relative to SEQ ID NO:4 when aligned by protocol 1 or protocol 2.

40. The non-naturally occurring polypeptide of claim 1, wherein the polypeptide is at least 75%, 80%, 85%, 90%, 91%, 92%, 93% 94%, 95%, 96%, 97%, 98%, or 99% identical to the N4 NA polypeptide of SEQ ID NO:4, and wherein the non-naturally occurring polypeptide includes 1, or both of the following amino acid residues relative to SEQ ID NO:4 when aligned by protocol 1 or protocol 2: 164Q/E, 195S.

41. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, 2, 3, 4, 5, 6, 7, 8, 9, 10, 11, 12, or all 13 of the following amino acid residues relative to SEQ ID NO:5 when aligned by protocol 1 or protocol 2:
97L, 410M, 96P, 98A, 100N, 102S/A, 110D, 128Q/M, 160I, 162V/A/I, 163V/P, 193Q/T, 445I; or
(b) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 2, 3, 4, 5, 6, 7, 8, 9, 10, 11, 12, 13, 14, 15, or all 16 of the following amino acid residues relative to SEQ ID NO:5 when aligned by protocol 1 or protocol 2:
97L, 410M, 96P, 98A, 100N, 102S/A, 103V, 110D, 128Q/M, 154T, 160I, 162V/A/I/T, 163V/P, 174I, 193Q/T, 445I.

42. The polypeptide of claim 41, wherein the polypeptide is
(a) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 1, 2, 3, 4, 5, 6, 7, 8, 9, 10, 11, 12, or all 13 of the following amino acid residues relative to SEQ ID NO:5 when aligned by protocol 1 or protocol 2:
(i) 97L, 410M, 96P, 98A, 100N, 102S/A, 110D, 128Q/M, 160I, 162V/A/I, 163V/P, 193Q/T, 445I; or
(ii) 96P, 98A, 100N, 102S/A, 110D, 128Q/M, 160I, 162V/A/I, 163V/P, 193Q/T, 445I.

43. The polypeptide of claim 41, wherein the polypeptide is
(b) at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes 2, 3, 4, 5, 6, 7, 8, 9, 10, 11, 12, 13, 14, 15, or all 16 of the following amino acid residues relative to SEQ ID NO:5 when aligned by protocol 1 or protocol 2:
(i) 97L, 410M, 96P, 98A, 100N, 102S/A, 103V, 110D, 128Q/M, 154T, 160I, 162V/A/I/T, 163V/P, 174I, 193Q/T, 445I; or
(ii) 96P, 98A, 100N, 102S/A, 103V, 110D, 128Q/M, 154T, 160I, 162V/A/I/T, 163V/P, 174I, 193Q/T, 445I.

44. The polypeptide of any one of claim 41-43, wherein the polypeptide includes one or more of the following sets of amino acid residues relative to SEQ ID NO:5 when aligned by protocol 1 or protocol 2:
(i)
97L/410M;
100N/102A;
98C/161C;
128Q/445I;
128M/445I;
98A/128M/445I/447A;
97L/98A/128M/445I/447A;
128M/445I/447A;
97L/128M/445I/447A;
96P/193T;
111I/163P;
96P/193T/202I;
96P/174I/193T; and/or
96P/174I/193T/202I; or
(ii)
100N/102A;
98C/161C;
128Q/445I;
128M/445I;
98A/128M/445I/447A;
97L/98A/128M/445I/447A;
128M/445I/447A;
97L/128M/445I/447A;
96P/193T;
111I/163P;
96P/193T/202I;
96P/174I/193T; and/or
96P/174I/193T/202I.

45. The polypeptide of any one of claim 41-43, wherein the polypeptide includes three or more of the listed amino acid residues relative to SEQ ID NO:5 when aliened by protocol 1 or protocol 2.

46. The polypeptide of any one of claim 41-43, wherein the polypeptide includes five or more of the listed amino acid residues relative to SEQ ID NO:5 when aligned by protocol 1 or protocol 2.

47. The polypeptide of any one of claim 41-43, wherein the polypeptide includes seven or more of the listed amino acid residues relative to SEQ ID NO:5 when aligned by protocol 1 or protocol 2.

48. The polypeptide of any one of claim 41-43, wherein the polypeptide includes nine or more of the listed ammo acid residues relative to SEQ ID NO:5 when aligned by protocol 1 or protocol 2.

49. The non-naturally occurring polypeptide of claim 1, wherein the polypeptide is at least 75%, 80%, 85%, 90%, 91%, 92%, 93%, 94%, 95%, 96%, 97%, 98%, or 99% identical to the N5 NA polypeptide of SEQ ID NO:5, and wherein the non-naturally occurring polypeptide includes I, or both of the following amino acid residues relative to SEQ ID NO:5 when aligned by protocol 1 or protocol 2: 162 Q/E, 200S.

50. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 1, 2, 3, 4, 5, 6, 7, 8, 9, 10, or all 11 of the following amino acid residues relative to SEQ ID NO:6 when aligned by protocol 1 or protocol 2:
99P, 103N, 113D, 131Q, 161I, 162P, 163I, 165S/T/V/A, 196Q, 203Y, 445S; or
(b) at least 75%, 80%, 85%, 90%, 91%, 92%, 93%, 94%g, 95%, 96%, 97%, 98%, or 99% identical to the N6 NA polypeptide of SEQ ID NO:6, and wherein the non-naturally occurring polypeptide includes 2, 3, 4, 5, 6, 7, 8, 9, 10, 11, 12, 13, 14, 15, or all 16 of the following amino acid residues relative to SF-Q ID NO:6 when aligned by protocol 1 or protocol 2:
99P, 103N, 105S, 106V, 113D, 131Q, 157T, 161I, 162P, 163I/L, 165S/T/V/A/I, 166V/P, 196Q, 203Y, 445S.

51. The polypeptide of claim 50, wherein the polypeptide is
(a) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 1, 2, 3, 4, 5, 6, 7, 8, 9, 10, or all 11 of the following amino acid residues relative to SEQ ID NO:6 when aligned by protocol 1 or protocol 2:
99P, 103N, 113D, 131Q, 161I, 162P, 163I, 165S/T/V/A, 196Q, 203Y, 445S.

52. The polypeptide of claim 50, wherein the polypeptide is
(b) at least 75%, 80%, 85%, 90%, 91%, 92%, 93%, 94%, 95%, 96%, 97%, 98%, or 99% identical to the N6 NA polypeptide of SEQ ID NO:6, and wherein the non-naturally occurring polypeptide includes 2, 3, 4, 5, 6, 7, 8, 9, 10, 11, 12, 13, 14, or all 15 of the following amino acid residues relative to SEQ ID NO:6 when aligned by protocol 1 or protocol 2:
99P, 103N, 105S, 106V, 113D, 131Q, 157T, 161I, 162P, 163I/L, 165S/T/V/A/I, 166V/P, 196Q, 203Y, 445S.

53. The polypeptide of any one of claim 50-52, wherein the polypeptide includes one or more of the following sets of amino acid residues relative to SEQ ID NO:6 when aligned by protocol 1 or protocol 2:
101C/164C;
101C/164C/174V;
101C/164C/174V/447V;
122V/447V;
101A/163L;
99P/196T; and/or
99P/196T/205I.

54. The polypeptide of any oik of claim 50-52, wherein the polypeptide includes three or more of the listed amino acid residues relative to SEQ ID NO:6 when aligned by protocol 1 or protocol 2.

55. The polypeptide of any one of claim 50-52, wherein the polypeptide includes five or more of the listed amino acid residues relative to SEQ ID NO:6 when aligned by protocol 1 or protocol 2.

56. The polypeptide of any one of claim 50-52, wherein the polypeptide includes seven or more of the listed amino acid residues relative to SEQ ID NO:6 when aligned by protocol 1 or protocol 2.

57. The polypeptide of any one of claim 50-52, wherein the polypeptide includes nine or more of the listed amino acid residues relative to SEQ ID NO:6 when aligned by protocol 1 or protocol 2.

58. The non-naturally occurring polypeptide of claim 1, wherein the polypeptide is at least 75%, 80%, 85%, 90%, 91%, 92%, 95%, 94%, 95%, 96%, 97%, 98%, or 99% identical to the N6 NA polypeptide of SEQ ID NO:6, and wherein the non-naturally occurring polypeptide includes a 165E amino acid mutation relative to SEQ ID NO:6 when aligned by protocol 1, and optionally also includes a 177V amino acid mutation relative to SEQ ID NO:6 when aligned by protocol 1 or protocol 2.

59. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 1, 2, 3, 4, 5, 6, 7, 8, 9, 10, or all 11 of the following amino acid residues relative to SEQ ID NO:7 when aligned by protocol 1 or protocol 2:
98P, 102N, 104S, 112D, 130Q, 156T, 162I, 164V/A/I, 165V, 202Y, 448V; or
(b) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 2, 3, 4, 5, 6, 7, 8, 9, 10, 11, 12, or all 13 of the following amino acid residues relative to SEQ ID NO:7 when aligned by protocol 1 or protocol 2:
98P, 102N, 104S, 112D, 130Q, 156T, 162I, 164V/A/I, 165V, 176I, 202Y, 446I, 448V.

60. The polypeptide of claim 59, wherein the polypeptide is
(a) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 1, 2, 3, 4, 5, 6, 7, 8, 9, 10, or all 11 of the following amino acid residues relative to SEQ ID NO:7 when aligned by protocol 1 or protocol 2:
98P, 102N, 104S, 112D, 130Q, 156T, 162I, 164V/A/I, 165V, 202Y, 448V.

61. The polypeptide of claim 59, wherein the polypeptide is
(b) at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes 2, 3, 4, 5, 6, 7, 8, 9, 10, 11, 12, or all 13 of the following amino acid residues relative to SEQ ID NO:7 when aligned by protocol 1 or protocol 2:
98P, 102N, 104S, 112D, 130Q, 156T, 162I, 164V/A/I, 165V, 176I, 202Y, 446I, 448V.

62. The polypeptide of any one of claim 59-61, wherein the polypeptide includes one or more of the following sets of amino acid residues relative to SEQ ID NO:7 when aligned by protocol 1 or protocol 2:
100C/163C;
100C/163C/173V;
100C/163C/173V/448V;
99L/446I/448A;
98P/195T;
130Q/446I;
446I/448A;
98P/195T/204I;
98P/176I/195T; and/or
98P/176I/195T/204I.

63. The polypeptide of any one of claim 59-61, wherein the polypeptide includes three or more of the listed amino acid residues relative to SEQ ID NO:7 when aligned by protocol 1 or protocol 2.

64. The polypeptide of any oik of claim 59-61, wherein the polypeptide includes five or more of the listed amino acid residues relative to SEQ ID NO:7 when aligned by protocol 1 or protocol 2.

65. The polypeptide of any one of claim 59-61, wherein the polypeptide includes seven or more of the listed amino acid residues relative to SEQ ID NO:7 when aligned by protocol 1 or protocol 2.

66. The polypeptide of any one of claim 59-61, wherein the polypeptide includes nine or more of the listed amino acid residues relative to SEQ ID NO:7 when aligned by protocol 1 or protocol 2.

67. The non-naturally occurring polypeptide of claim 1, wherein the polypeptide is at least 75%, 80%, 85%, 90%, 91%, 92%, 93%, 94%, 95%, 96%, 97%, 98%, or 99% identical to the N7 NA polypeptide of SEQ ID NO:7, and wherein the non-naturally occurring polypeptide includes one or both of the following amino acid mutation relative to SEQ ID NO:7 when aligned by protocol 1 or protocol 2: 164Q/E, 195S.

68. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 1, 2, 3, 4, 5, 6, 7, 8, 9, 10, or all 11 of the following amino acid residues relative to SEQ ID NO:8 when aligned by protocol 1 or protocol 2:
408M, 101N, 103A/S, 111D, 129Q, 161P/L, 63S/T/V/A/I, 164V, 194Q, 201Y, 442I; or
(b) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 2, 3, 4, 5, 6, 7, 8, 9, 10, 11, 12, 13, 14, or all 15 of the following amino acid residues relative to SEQ ID NO:8 when aligned by protocol 1 or protocol 2:
98L, 408M, 101N, 103A/S, 104V, 111D, 129Q/M, 161P/L, 163S/T/V/A/I/S/T, 164V/P, 175I, 194Q, 201Y, 203I, 442I

69. The polypeptide of claim 68, wherein the polypeptide is
(a) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 1, 2, 3, 4, 5, 6, 7, 8, 9, 10, or all 11 of the following amino acid residues relative to SEQ ID NO:8 when aligned by protocol 1 or protocol 2:
(i) 408M, 101N, 103A/S, 111D, 129Q, 160P, 161L, 163S/T/V/A/I, 164V, 194Q, 201Y, 442I; or
(ii) 101N, 103A/S, 111D, 129Q, 160P, 161L, 163S/T/V/A/I, 164V, 194Q, 201Y, 442I.

70. The polypeptide of claim 68, wherein the polypeptide is
(b) at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes 2, 3, 4, 5, 6, 7, 8, 9, 10, 11, 12, 13, 14, or all 15 of the following amino acid residues relative to SEQ ID NO:8 when aligned by protocol 1 or protocol 2:
(i) 98L, 408M, 101N, 103A/S, 104V, 111D, 129Q/M, 160P, 161L, 163S/T/V/A/I/S/T, 164V/P, 175I, 194Q, 201Y, 203I, 442I; or
(ii) 101N, 103A/S, 104V, 111D, 129Q/M, 160P, 161L, 163S/T/V/A/I/S/T, 164V/P, 175I, 194Q, 201Y, 203I, 442I

71. The polypeptide of any one of claim 68-70, wherein the polypeptide includes one or more of the following sets of amino acid residues relative to SEQ ID NO:8 when aligned by protocol 1 or protocol 2:
(i)
98L/408M;
160P/163S;
101N/103A;
99C/162C;
99C/162C/172V;
129Q/442I;
99A/161L;
99A/161L/442I;
161L/442I;
129M/442I;
99A/129M/161L/442I/444A;
98L/99A/129M/161L/442I/444A;
129M/161L/442I/444A;
98L/129M/161L/442I/444A; and/or
175I/203I; or
(ii)
160P/163S;
101N/103A;
99C/162C;
99C/162C/172V;
129Q/442I;
99A/161L;
99A/161L/442I;
161L/442I;
129M/442I
99A/129M/161L/442I/444A;
98L/99A/129M/161L/442I/444A;
129M/161L/442I/444A;
98L/129M/161L/442I/444A; and or
175I/203I.

72. The polypeptide of any one of claim 68-70, wherein the polypeptide includes three or more of the listed amino acid residues relative to SEQ ID NO:8 when aligned by protocol 1 or protocol 2.

73. The polypeptide of any one of claim 68-70, wherein the polypeptide includes five or more of the listed amino acid residues relative to SEQ ID NO:8 when aligned by protocol 1 or protocol 2.

74. The polypeptide of any one of claim 68-70, wherein the polypeptide includes seven or more of the listed amino acid residues relative to SEQ ID NO:8 when aligned by protocol 1 or protocol 2.

75. The polypeptide of any oik of claim 68-70, wherein the polypeptide includes nine or more of the listed amino acid residues relative to SEQ ID NO:8 when aligned by protocol 1 or protocol 2.

76. The non-naturally occurring polypeptide of any one of claims 80-85, wherein the polypeptide comprises the amino acid sequence at least 75%, 80%, 85%, 90%, 91%, 92%, 93%, 94%, 95%, 96%, 97%, 98%, or 99% identical to the amino acid selected from the group consisting of N8 mutants listed in Table 1 (SEQ ID NO:35-38), when aligned by protocol 1, wherein the polypeptide includes all of the residues listed in Table 1 for an individual N8 mutant listed in Table 1.

77. The non-naturally occurring polypeptide of claim 1, wherein the polypeptide is at least 75%, 80%, 85%, 90%, 91%, 92%, 93%, 94%, 95%, 96%, 97%, 98%, or 99% identical to the N8 NA polypeptide of SEQ ID NO:8, and wherein the non-naturally occurring polypeptide includes a 163E amino acid mutation relative to SEQ ID NO: 8 when aligned by protocol 1, and further may optionally include a 194S mutation relative to SEQ ID NO:8 when aligned by protocol 1 or protocol 2.

78. The non-naturally occurring polypeptide of claim 1, wherein the polypeptide is
(a) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 1, 2, 3, 4, 5, 6, 7, 8, 9, or all 10 of the following amino acid residues relative to SEQ ID NO:9 when aligned by protocol 1 or protocol 2:
94P, 95L, 100S, 126Q/M, 160V/A/I, 161V, 191Q, 198Y, 439S, 441V; or
(b) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 2, 3, 4, 5, 6, 7, 8, 9, 10, 11, 12, 13, or all 14 of the following amino acid residues relative to SEQ ID NO:9 when aligned by protocol 1 or protocol 2:
94P, 95L, 98N, 100S/A, 126Q/M, 152T, 158I, 160V/A/I/T, 161V, 191Q, 198Y, 200I, 439S, 441V.

79. The polypeptide of claim 78, wherein the polypeptide is
(a) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 1, 2, 3, 4, 5, 6, 7, 8, 9, or all 10 of the following amino acid residues relative to SEQ ID NO:9 when aligned by protocol 1 or protocol 2:
94P, 95L, 100S, 126Q/M, 160V/A/I, 161V, 191Q, 198Y, 439S, 441V.

80. The polypeptide of claim 78, wherein the polypeptide is
(b) at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes 2, 3, 4, 5, 6, 7, 8, 9, 10, 11, 12, 13, or all 14 of the following amino acid residues relative to SEQ ID NO:9 when aligned by protocol 1 or protocol 2:
94P, 95L, 98N, 100S/A, 126Q/M, 152T, 158I, 160V/A/I/T, 161V, 191Q, 198Y, 200I, 439S, 441V.

81. The polypeptide of any one of claim 78-80, wherein the polypeptide includes one or more of the following sets of amino acid residues relative to SEQ ID NO:9 when aligned by protocol 1 or protocol 2:
96C/159C;
96C/159C/441V;
96A/441A;
96A/126M/439I/441A;
95L/96A/126M/439I/441A;
126M/439I/441A;
95L/126M/439I/441A;
94P/191T;
98N/100A; and/or
94P/191T/200I.

82. The polypeptide of any one of claim 78-80, wherein the polypeptide includes three or more of the listed amino acid residues relative to SEQ ID NO:9 when aligned by protocol 1 or protocol 2.

83. The polypeptide of any one of claim 78-80, wherein the polypeptide includes five or more of the listed amino acid residues relative to SEQ ID NO:9 when aligned by protocol 1 or protocol 2.

84. The polypeptide of any oik of claim 78-80, wherein the polypeptide includes seven or more of the listed amino acid residues relative to SEQ ID NO:9 when aligned by protocol 1 or protocol 2.

85. The polypeptide of any one of claim 78-80, wherein the polypeptide includes nine or more of the listed amino acid residues relative to SEQ ID NO:9 when aligned by protocol 1 or protocol 2.

86. The non-naturally occurring polypeptide of claim 1, wherein the polypeptide is at least 75%, 80%, 85%, 90%, 91%, 92%, 93%, 94%, 95%, 96%, 97%, 98%, or 99% identical to the N9 NA polypeptide of SEQ ID NO:9, and wherein the non-naturally occurring polypeptide includes a 160Q amino acid mutation relative to SEQ ID NO:9 when aligned by protocol 1, or may include a combination of 160Q/E and 172V amino acid residues relative to SEQ ID NO:9 when aligned by protocol 1 or protocol 2.

87. A composition, comprising one or more of the polypeptides of any preceding claim linked to a scaffold.

88. The composition of claim 87, wherein the scaffold comprises a protein scaffold.

89. The composition of claim 88, wherein the polypeptide is covalently linked to a protein subunit of the protein scaffold to form a fusion protein.

90. A nucleic acid encoding the polypeptide of any preceding claim, or the fusion protein of claim 89.

91. An expression vector comprising the nucleic acid of claim 90 operatively linked to a suitable control sequence.

92. A host cell comprising the nucleic acid of claim 90, the expression vector of claim 91, and or the polypeptide of any preceding claim.

93. A pharmaceutical composition, comprising
(a) one or more of the polypeptides, fusion protein, composition, nucleic acid, expression vector, and or the host cell of any preceding claim, and
(b) a pharmaceutically acceptable carrier.

94. A vaccine comprising
(a) one or more of the polypeptides, fusion protein, composition, nucleic acid, expression vector, and or the host cell of any preceding claim; and
(b) a pharmaceutically acceptable carrier.

95. A method for treating or limiting development of an influenza infection, comprising administering to a subject in need thereof an amount effective to treat or limit development of the influenza infection of a polypeptide, fusion protein, composition, vaccine, nucleic acid, expression vector, host cell, pharmaceutical composition, and/or vaccine of any preceding claim.
